(19) United States
(12) Patent Application Publication
(10) Pub. No.: US 2020/0339567 A1
(43) Pub. Date: Oct. 29, 2020

(54) SUBSTITUTED IMIDAZOPYRIDINE AMIDES AND USE THEREOF

(71) Applicants: Bayer Aktiengesellschaft, Leverkusen (DE); Bayer Pharma Aktiengesellschaft, berlin (DE)

(72) Inventors: Daniel MEIBOM, Wuppertal (DE); Jutta MEYER, Solingen (DE); Karl COLLINS, Düsseldorf (DE); Nuria ORTEGA HERNANDEZ, Wuppertal (DE); Jan STAMPFUSS, Düsseldorf (DE); Frank WUNDER, Wuppertal (DE); Till FREUDENBERGER, Velbert (DE); Thomas MONDRITZKI, Essen (DE); Nina Alexandra SCHEERER, Castrop-Rauxel (DE); Kirsten LEINEWEBER, Velbert-Langenberg (DE); Jens SCHAMBERGER, Velbert-Langenberg (DE); Alexander STRAUB, Wuppertal (DE); Kersten Matthias GERICKE, Wuppertal (DE); Walter KROH, Wuppertal (DE); Mario LOBELL, Wuppertal (DE); Klaus MÜNTER, Wülfrath (DE)

(73) Assignees: Bayer Aktiengesellschaft, Leverkusen (DE); Bayer Pharma Aktiengesellschaft, Berlin (DE)

(21) Appl. No.: 16/758,751

(22) PCT Filed: Oct. 18, 2018
(86) PCT No.: PCT/EP2018/078653

§ 371 Date: Apr. 23, 2020

(30) Foreign Application Priority Data
Oct. 24, 2017 (EP) 17198021.2

(51) Int. Cl. C07D 471/04; C07D 519/00; A61K 31/444; A61K 45/06; A61P 9/10; A61K 31/437

(52) U.S. Cl. CPC C07D 471/04; C07D 519/00; A61K 31/437; A61K 45/06; A61P 9/10; A61K 31/444

(57) ABSTRACT

The present application relates to novel substituted imidazopyridine amides of the formula (I), to processes for their preparation, to their use, alone or in combinations, for the treatment and/or prophylaxis of diseases and to their use for the production of medicaments for the treatment and/or prophylaxis of diseases, in particular for the treatment and/or prophylaxis of cardiovascular, neurological and central nervous as well as metabolic disorders.

The present application relates to novel substituted imidazopyridine amides, to processes for their preparation, to their use, alone or in combinations, for the treatment and/or prophylaxis of diseases and to their use for the production of medicaments for the treatment and/or prophylaxis of diseases, in particular for the treatment and/or prophylaxis of cardiovascular, neurological and central nervous as well as metabolic disorders.

The α-2B adrenoreceptor (ADRA2B) belongs to the group of the adrenoreceptors which are activated by the natural transmitters adrenaline and noradrenaline and are therefore responsible for the effects mediated by adrenaline and noradrenaline. The α-2B adrenoreceptor is a G-protein-coupled receptor (GPCR) associated with the inhibitory Gαi signal pathway.

The receptor is expressed centrally in the brain and peripherally on smooth vascular muscle cells and mediates centrally sodium retention and peripheral vasoconstriction (Am J Physiol Regulatory Integrative Comp Physiol. 2002; 283: R287-295). It is also highly expressed in the kidney (Clin Sci (Lond). 2005; 109(5):431-7) where it may have a possible role in renal perfusion and diuresis (International Journal of Cardiology 2004; 97:367-372).

As is the case with many G-protein-coupled receptors, with ADRA2B, too, many endogenous agonists induce a GRK(G-protein receptor kinase)-dependent phosphorylation leading to desensitization and internalization of the receptor. In the case of prolonged stimulation of the receptor by the agonist, this desensitization and internalization of the receptor leads to reduced activation of the downstream signal cascade (G-protein activation) and thus to a reduced responsiveness of the cell to the agonist. In the genetic DD variant of the ADRA2B, there is a deletion of 3 glutamic acids in the 3rd intracellular loop of the receptor, which reduces agonist-induced receptor phosphorylation and desensitization. This results in a prolonged activation of the receptor and the signal cascade following agonist stimulation (Cell Commun Signal. 2011; 9(1):5).

A number of studies have shown a significant association of the ADRA2B DD variant with the occurrence of certain disorders. In the normal population, depending on ethnicity, 20-30% of people carry the DD variant of the receptor. In patients suffering from cardiac disorders, the proportion of people carrying the DD variant increases to almost 50%. Thus, the DD variant is significantly associated with the occurrence of myocardial infarction and sudden heart death in man (J Am Coll Cardiol. 2003; 41(2):190-4; J Am Coll Cardiol. 2001; 37(6):1516-22). Based on in vitro findings of a prolonged activity of the DD variant, the DD variant is thought to lead, via prolonged receptor activation, to a reduced function of small coronary vessels and endothelial dysfunction (Clin Sci (Lond). 2002; 103(5):517-24; Clin Sci (Lond). 2003; 104(5):509-20). Accordingly, the DD genotype of ADRA2B is considered to be a genetic risk factor for the above disorders.

Furthermore, the DD variant of ADRA2B is significantly associated with the occurrence of ischaemic strokes. This, too, appears to be based on a functional disturbance of the small vessels (Clin Neurol Neurosurg. 2013; 115(1):26-31). These association studies (genetic data) point to a pathomechanistic relevance of the ADRA2B receptor—independently of the genotype—for ischaemic disorders, in particular ischaemic heart disorders.

Also associated with the DD variant of ADRA2B is the occurrence of posttraumatic stress disorders (PTSD) caused by the enhanced recollection of traumatic events (Nat Neurosci. 2007; 10(9):1137-9; Neurobiol Learn Mem. 2014; 112:75-86). As a neurotransmitter, noradrenaline is involved in the processing of emotional memory processes. The DD variant of the ADRA2B receptor is presumably the result of an increased effect of noradrenaline as a response to emotional events, leading to enhanced amygdala activation and increased emotional recollection. In patients suffering from PTSD, an increased amygdala activation correlates with the severity of the symptoms. (Li et al., Psychopharmacology 2015; Rasch et al PNAS 2009; van Stegeren, Acta Psychologica, 2008). These effects are mediated by central ADRA2B receptors and noradrenergic signal transduction influenced thereby.

Also, it was possible to demonstrate association of the DD variant with the early onset of type 2 diabetes (Exp Clin Endocrinol Diabetes 2006; 114: 424-427).

Accordingly, inhibition of the ADRA2B receptor represents a promising treatment option for cardiovascular, neurological and central nervous as well as metabolic disorders.

In the field of cardiovascular disorders, there is a great demand for novel treatment methods. Even with the therapy currently available, morbidity and mortality after myocardial infarction are still high. Even in the case of rapid reopening of the coronary vessels (reperfusion, percutaneous coronary intervention (PCI)), mortality as a result of myocardial infarction is high: 7%-11% of the patients die as a consequence of the infarction, and within a year 22% of the patients have to attend a hospital owing to heart failure as a consequence of the infarction (Freisinger et al., European Heart Journal (2014) 35, 979-988).

Disruption of blood flow during a myocardial infarction leads to cell death in the region of the area supplied by the coronary vessel in question. It is generally accepted that re-opening of the occluded vessel and thus restoration of blood flow is vital for saving the heart tissue affected; however, paradoxically, the restored blood flow, too, leads to tissue damage which counteracts the original advantage of reperfusion. 50% of the final size of the infarction can be ascribed to this reperfusion damage (Fröhlich et al, European Heart Journal 2013, 34). Disturbed blood flow in small coronary vessels (microvascular dysfunction), despite re-opening of the original occlusion in the epicardial vessel, contributes to reperfusion damage and thus to the final infarction size.

Novel therapeutic strategies for reducing infarction size and for maintaining cardiac function are required to improve patient survival and to prevent heart failure after myocardial infarction.

It was an object of the present invention to identify and provide novel low-molecular-weight compounds which act as potent antagonists of the ADRA2B receptor and are thus suitable for treatment and/or prevention of cardiovascular, neurological and central nervous as well as metabolic disorders.

A further object consists in the identification of ADRA2B antagonists for use in myocardal infarction patients, in particular for reducing reperfusion damage.

ADRA2B inhibitors are described, for example, in WO 03/008387 and in WO2010/033393. WO2009/47506 and WO2009/47522 disclose imidazopyridinecarboxamides as tyrosine kinase inhibitors.

EP 1277754 discloses imidazopyridine derivatives which act as phosphatidylinositol 3-kinase (PI3K) inhibitors and can thus be employed as antitumour agents.

WO 2008/027812 discloses imidazopyridines and imidazopyrimidine derivatives which act as cannabinoid receptor ligands, e.g. CB2 ligands.

WO 2008/134553 describes bicyclic compounds which may be used, inter alia, for treating pain.

Imidazopyridine derivatives as modulators of TNF activity are described in WO 2014/009295.

However, the prior art does not describe the imidazopyridine amides of the general formula (I) of the present invention described and defined here.

It has now been found the the compounds of the present invention have surprising and advantageous properties which achieve the object of the present invention.

In particular, it has been found that the compounds of the present invention are ADRA2B antagonists. In particular, by virtue of their good solubility, the compounds according to the invention are suitable for parenteral administration forms (European Pharmacopoeia, 6th Edition, initial volumes (Ph.Eur. 6.0), p. 1024), thus making accessible novel treatment options. Accordingly, the compounds mentioned are suitable in particular for acute therapy, for example acute administration during percutaneous coronary intervention, and also for other acute situations which may lead to hypoperfusion and organ damage (heart, kidney, brain).

The present invention provides compounds of the formula (I)

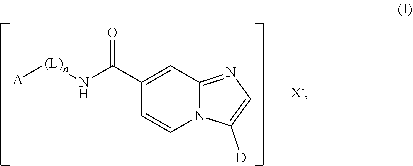

in which

A represents a positively charged aza heteroaromatic of the formula

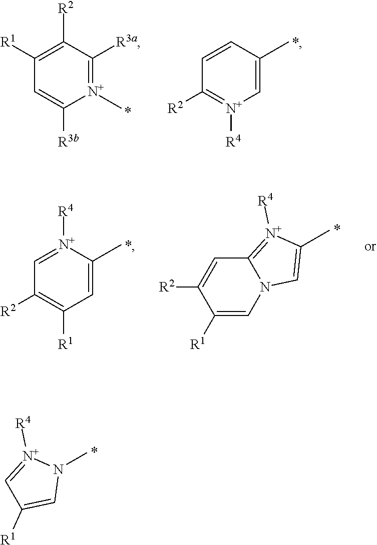

in which

* represents the point of attachment,

R¹, R², and R^{3a}, R^{3b} independently of one another represent a radical selected from the group consisting of hydrogen, amino, (C₁-C₄)-alkyl, (C₁-C₄)-alkoxy, mono-(C₁-C₄)-alkylamino, di-(C₁-C₄)-alkylamino, phenoxy and piperidin-1-yl, where phenoxy and piperidin-1-yl may be substituted by (C₁-C₄)-alkyl and/or fluorine and where the alkylgroupsin(C₁-C₄)-alkyl, (C₁-C₄)-alkoxy, mono-(C₁-C₄)-alkylamino and di-(C₁-C₄)-alkylamino may each be up to pentasubstituted by fluorine,

R⁴ represents (C₁-C₄)-alkyl which may be up to pentasubstituted by fluorine, or represents a group of the formula CH₂CN, CH₂CONH₂, D represents a heteroaromatic of the formula

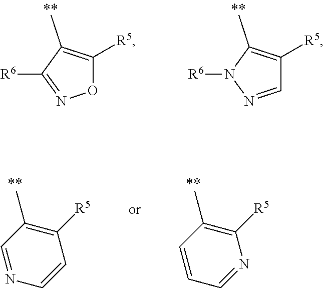

in which

** represents the point of attachment,

R⁵ and R⁶ independently of one another represent hydrogen, (C₁-C₄)-alkyl or (C₁-C₄)-alkoxy, where (C₁-C₄)-alkyl and (C₁-C₄)-alkoxy may each be up to pentasubstituted by fluorine,

L represents CH₂,

n represents the number 0, 1, 2 or 3 and

X⁻ represents a physiologically acceptable anion,

and the solvates, salts and solvates of the salts of the compounds of the formula (I).

The present invention also encompasses expedient forms of the compounds of the present invention such as metabolites, hydrates, solvates, prodrugs, salts, in particular pharmaceutically acceptable salts, and/or coprecipitates.

The compounds of the formula (I) according to the invention are already present in salt form; however, they may form further addition salts. Compounds of the invention are the compounds of the formula (I) and the salts, solvates and solvates of the salts thereof, the compounds that are encompassed by formula (I) and are of the formulae mentioned below and the salts, solvates and solvates of the salts thereof and the compounds that are encompassed by formula (I) and are cited below as working examples and the salts, solvates and solvates of the salts thereof if the compounds that are encompassed by formula (I) and are mentioned below are not already salts, solvates and solvates of the salts.

Preferred salts in the context of the present invention are physiologically acceptable salts of the compounds of the invention. Also encompassed are salts which are not themselves suitable for pharmaceutical applications but can be used, for example, for isolation or purification of the compounds of the invention.

The term “pharmaceutically acceptable salt” refers to an inorganic or organic acid addition salt of a compound according to the present invention. See, for example, S. M. Berge, et al. “Pharmaceutical Salts”, J. Pharm. Sci. 1977, 66, 1-19.

A suitable pharmaceutically acceptable salt of the compounds of the present invention may be, for example, an acid-addition salt of a compound of the present invention, such as an acid-addition salt with an inorganic acid, or “mineral acid”, such as hydrochloric acid, hydrofluoric acid, hydrobromic acid, hydroiodic acid, sulfuric acid, sulfamic acid, bisulfuric acid, phosphoric acid or nitric acid, for example, or with an organic acid such as formic acid, acetic acid, acetoacetic acid, pyruvic acid, trifluoroacetic acid, propionic acid, butyric acid, hexanoic acid, heptanoic acid, undecanoic acid, lauric acid, benzoic acid, salicylic acid, 2-(4-hydroxybenzoyl)benzoic acid, camphoric acid, cinnamic acid, cyclopentanepropionic acid, digluconic acid, 3-hydroxy-2-naphthoic acid, nicotinic acid, pamoic acid, pectinic acid, 3-phenylpropionic acid, pivalic acid, 2-hydroxyethanesulfonic acid, itaconic acid, trifluoromethanesulfonic acid, dodecylsulfuric acid, ethanesulfonic acid, benzenesulfonic acid, para-toluenesulfonic acid, methanesulfonic acid, 2-naphthalenesulfonic acid, naphthalenedisulfonic acid, camphorsulfonic acid, citric acid, tartaric acid, stearic acid, lactic acid, oxalic acid, malonic acid, succinic acid, malic acid, adipic acid, alginic acid, maleic acid, fumaric acid, D-gluconic acid, mandelic acid, ascorbic acid, glucoheptanoic acid, glycerophosphoric acid, aspartic acid, sulfosalicylic acid or thiocyanic acid, for example.

Those skilled in the art will further recognize that it is possible for acid addition salts of the claimed compounds to be prepared by reaction of the compounds with the appropriate inorganic or organic acid via any of a number of known methods. The present invention includes all possible salts of the compounds of the present invention as single salts, or as any mixture of said salts, in any ratio.

Physiologically acceptable anions in the context of the present invention are the anions of mineral acids, carboxylic acids and sulfonic acids, for example salts of hydrochloric acid, hydrofluoric acid, hydrobromic acid, hydroiodic acid, sulfuric acid, phosphoric acid, methanesulfonic acid, ethanesulfonic acid, toluenesulfonic acid, benzenesulfonic acid, naphthalenedisulfonic acid, formic acid, acetic acid, trifluoroacetic acid, propionic acid, lactic acid, tartaric acid, malic acid, citric acid, fumaric acid, maleic acid and benzoic acid. Preference is given to the anions of the following acids: hydrochloric acid, hydrobromic acid, formic acid. Particular preference is given to the anions of hydrochloric acid, hydrobromic acid and formic acid.

Solvates in the context of the invention are described as those forms of the compounds of the invention which form a complex in the solid or liquid state by coordination with solvent molecules. Hydrates are a specific form of the solvates in which the coordination is with water. Solvates preferred in the context of the present invention are hydrates.

The compounds according to the invention may, depending on their structure, exist in different stereoisomeric forms, i.e. in the form of configurational isomers or else, if appropriate, as conformational isomers (enantiomers and/or diastereomers, including those in the case of atropisomers). The present invention therefore encompasses the enantiomers and diastereomers, and the respective mixtures thereof. The stereoisomerically homogeneous constituents can be isolated in a known manner from such mixtures of enantiomers and/or diastereomers; preference is given to using chromatographic methods for this purpose, in particular HPLC chromatography on an achiral or chiral phase. In the case of carboxylic acids as intermediates or end products, separation is alternatively also possible via diastereomeric salts using chiral amine bases.

If the compounds of the invention can occur in tautomeric forms, the present invention encompasses all the tautomeric forms.

In the compounds of the formula (I) according to the invention, the positively charged aza heteroaromatics can, in addition to formula A shown, also be present in the respective contributing mesomeric structures comprised by A, in particular the following contributing structure:

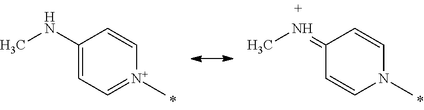

The compounds of the general formula (I) may take the form of isotopic variants. The invention therefore encompasses one or more isotopic variants of the compounds of the general formula (I), especially deuterium-containing compounds of the general formula (I).

The term “isotopic variant” of a compound or reagent is defined as a compound with an unnatural fraction of one or more isotopes from which such a compound is constituted.

The term “isotopic variant of the compound of the general formula (I)” is defined as a compound of the general formula (I) with an unnatural proportion of one or more isotopes from which such a compound is formed.

The expression “unnatural fraction” is understood to mean a fraction of such an isotope higher than its natural frequency. The natural frequencies of isotopes to be employed in this connection can be found in “Isotopic Compositions of the Elements 1997”, Pure Appl. Chem., 70(1), 217-235, 1998.

Examples of such isotopes are stable and radioactive isotopes of hydrogen, carbon, nitrogen, oxygen, phosphorus, sulphur, fluorine, chlorine, bromine and iodine, such as ²H (deuterium), ³H (tritium), ¹¹C, ¹³C, ¹⁴C, ¹⁵N, ¹⁷O, ¹⁸O, ³²P, ³³P, ³³S, ³⁴S, ³⁵S, ³⁶S, ¹⁸F, ³⁶C, ⁸²Br, ¹²³I, ¹²⁴I, ¹²⁵I, ¹²⁹I and ¹³¹I.

With regard to the treatment and/or prophylaxis of the disorders specified here, the isotopic variant(s) of the compounds of the general formula (I) preferably contain deuterium (“deuterium-containing compounds of the general formula (I)”). Isotopic variants of the compounds of the general formula (I) into which one or more radioactive isotopes such as 3H or ¹⁴C have been incorporated are beneficial, for example, in medicament and/or substrate tissue distribution studies. Because of their easy incorporability and detectability, these isotopes are particularly preferred. It is possible to incorporate positron-emitting isotopes such as ¹⁸F or ¹¹C into a compound of the general formula (I). These isotopic variants of the compounds of the general formula (I) are suitable for use in in vivo imaging applications. Deuterium-containing and ¹³C-containing compounds of the general formula (I) can be used within preclinical or clinical studies in mass spectrometry analyses.

Isotopic variants of the compounds of the general formula (I) can generally be prepared by processes known to those skilled in the art as described in the schemes and/or examples described here, by replacing a reagent with an isotopic variant of the reagent, preferably a deuterium-containing reagent. According to the desired deuteration sites, in some cases, deuterium from D₂O can either be incorporated directly into the compounds or into reagents which can be used for the synthesis of such compounds. Another useful reagent for incorporation of deuterium into molecules is deuterium gas. A rapid route to the incorporation of deuterium is the catalytic deuteration of olefinic bonds and acetylenic bonds. For direct exchange of hydrogen for deuterium in hydrocarbons containing functional groups, it is also possible to use metal catalysts (i.e. Pd, Pt and Rh) in the presence of deuterium gas. Various deuterated reagents and synthesis units are commercially available from companies like, for example, C/D/N Isotopes, Quebec, Canada; Cambridge Isotope Laboratories Inc., Andover, Mass., USA; and CombiPhos Catalysts, Inc., Princeton, N.J., USA.

The term “deuterium-containing compound of the general formula (I)” is defined as a compound of the general formula (I) in which one or more hydrogen atoms have been replaced by one or more deuterium atoms and in which the frequency of deuterium in every deuterated position in the compound of the general formula (I) is higher than the natural frequency of deuterium, which is about 0.015%. More particularly, in a deuterium-containing compound of the general formula (I), the frequency of deuterium in every deuterated position in the compound of the general formula (I) is higher than 10%, 20%, 30%, 40%, 50%, 60%, 70% or 80%, preferably higher than 90%, 95%, 96% or 97%, even further preferably higher than 98% or 99%, in this position or these positions. It will be apparent that the frequency of deuterium in every deuterated position is independent of the frequency of deuterium in other deuterated positions.

Through the selective incorporation of one or more deuterium atoms into a compound of the general formula (I), it is possible to alter the physicochemical properties (for example acidity [C. L. Perrin, et al., J. Am. Chem. Soc., 2007, 129, 4490], basicity [C. L. Perrin et al., J. Am. Chem. Soc., 2005, 127, 9641], lipophilicity [B. Testa et al., Int. J. Pharm., 1984, 19(3), 271]) and/or the metabolic profile of the molecule and cause changes in the ratio of parent compound to metabolites or the amounts of metabolites formed.

Such changes may lead to particular therapeutic benefits and therefore be preferable under particular circumstances. Reduced rates of metabolism and metabolic switching, where the ratio of metabolites is changed, have been reported (A. E. Mutlib et al., Toxicol. Appl. Pharmacol., 2000, 169, 102). These changes in the exposure to parent compound and metabolites can have important consequences with respect to the pharmacodynamics, tolerability and efficacy of a deuterium-containing compound of the general formula (I). In some cases deuterium substitution reduces or eliminates the formation of an undesired or toxic metabolite and enhances the formation of a desired metabolite (e.g. Nevirapine: A. M.

Sharma et al., Chem. Res. Toxicol., 2013, 26, 410; Efavirenz: A. E. Mutlib et al., Toxicol. Appl. Pharmacol., 2000, 169, 102). In other cases the major effect of deuteration is to reduce the rate of systemic clearance. As a result, the biological half-life of the compound is increased. The potential clinical benefits would include the ability to maintain similar systemic exposure with decreased peak levels and increased trough levels. This could result in lower side effects and enhanced efficacy, depending on the particular compound's pharmacokinetic/pharmacodynamic relationship. Examples of this deuterium effect are ML-337 (C. J. Wenthur et al., J. Med. Chem., 2013, 56, 5208) and odanacatib (K. Kassahun et al., WO2012/112363). Still other cases have been reported in which reduced rates of metabolism result in an increase in exposure of the drug without changing the rate of systemic clearance (e.g. Rofecoxib: F. Schneider et al., Arzneim. Forsch./Drug. Res., 2006, 56, 295; Telaprevir: F. Maltais et al., J. Med. Chem., 2009, 52, 7993). Deuterated drugs showing this effect may have reduced dosing requirements (e.g. lower number of doses or lower dosage to achieve the desired effect) and/or may produce lower metabolite loads.

A compound of general formula (I) may have multiple potential sites of attack for metabolism. To optimize the above-described effects on physicochemical properties and metabolic profile, deuterium-containing compounds of general formula (I) having a certain pattern of one or more deuterium-hydrogen exchange(s) can be selected. Particularly, the deuterium atom(s) of deuterium-containing compound(s) of general formula (I) is/are attached to a carbon atom and/or is/are located at those positions of the compound of general formula (I), which are sites of attack for metabolizing enzymes such as e.g. cytochrome P₄₅₀.

The present invention additionally also encompasses prodrugs of the compounds according to the invention. The term “prodrugs” in this context refers to compounds which may themselves be biologically active or inactive but are reacted (for example metabolically or hydrolytically) to give compounds of the invention during their residence time in the body.

In the context of the present invention, unless specified otherwise, the substituents are defined as follows:

In the context of the invention, alkyl or (C₁-C₄)-alkyl is a straight-chain or branched alkyl radical having 1 to 4 carbon atoms. Preferred examples include: methyl, ethyl, n-propyl, isopropyl, n-butyl, isobutyl, 1-methylpropyl, tert-butyl. Preference is given to methyl, ethyl and isopropyl. Particular preference is given to methyl.

In the context of the invention, alkoxy or (C₁-C₄)-alkoxy is a straight-chain or branched alkoxy radical having 1 to 4 carbon atoms. Preferred examples include: methoxy, ethoxy, n-propoxy, isopropoxy, n-butoxy and tert-butoxy. Preference is given to methoxy and ethoxy. Particular preference is given to methoxy.

In the context of the present invention, mono-(C₁-C₄)-alkylamino is an amino group having a straight-chain or branched alkyl substituent having 1 to 4 carbon atoms. Preferred examples include the following: methylamino, ethylamino, n-propylamino, isopropylamino, n-butylamino, sec-butylamino and tert-butylamino. Particular preference is given to methylamino.

In the context of the present invention, di-(C₁-C₄)-alkylamino is an amino group having two identical or different straight-chain or branched alkyl substituents each having 1 to 4 carbon atoms. Preferred examples include the following: N,N-dimethylamino, N,N-diethylamino, N-ethyl-N-methylamino, N-methyl-N-n-propylamino, N-isopropyl-N-methylamino, N-isopropyl-N-n-propylamino, N,N-diisopropylamino, N-n-butyl-N-methylamino and N-tert-butyl-N-methylamino. Particular preference is given to dimethylamino.

When radicals in the compounds of the invention are substituted, the radicals may be mono- or polysubstituted, unless specified otherwise. In the context of the present invention, all radicals which occur more than once are defined independently of one another. Substitution by one or two identical or different substituents is preferred. Very particular preference is given to substitution by one substituent.

In the context of the present invention, the term “treatment” or “treating” includes inhibition, retardation, checking, alleviating, attenuating, restricting, reducing, suppressing, repelling or healing of a disease, a condition, a disorder, an injury or a health problem, or the development, the course or the progression of such states and/or the symptoms of such states. The term “therapy” is understood here to be synonymous with the term “treatment”.

The terms “prevention”, “prophylaxis” and “preclusion” are used synonymously in the context of the present invention and refer to the avoidance or reduction of the risk of contracting, experiencing, suffering from or having a disease, a condition, a disorder, an injury or a health problem, or a development or advancement of such states and/or the symptoms of such states.

The treatment or prevention of a disease, a condition, a disorder, an injury or a health problem may be partial or complete.

In the context of the present invention, preference is given to compounds of the general formula (I) in which

R¹, R², and R^{3a}, R^{3b} independently of one another represent a group selected from hydrogen, ethylamino, dimethylamino, methylamino, amino, methyl, ethyl, trifluoromethyl, t-butyl, isopropyl, phenoxy or piperidin-1-yl,

R⁴ represents methyl,

R⁵ and R⁶ independently of one another represent hydrogen, methyl, ethyl, isopropyl or methoxy,

n represents the number 1 or 2,

X⁻ represents bromide, chloride or formate and

A represents a positively charged aza heteroaromatic of the formula

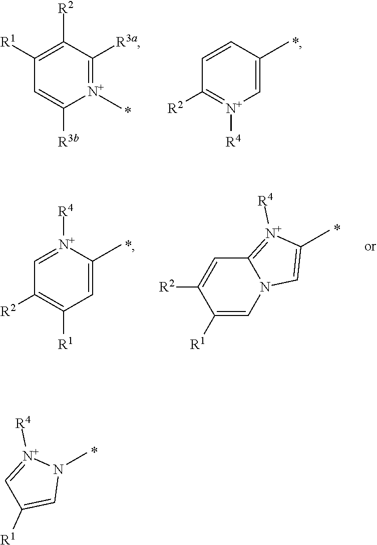

in which

* represents the point of attachment,

D represents a heteroaromatic of the formula

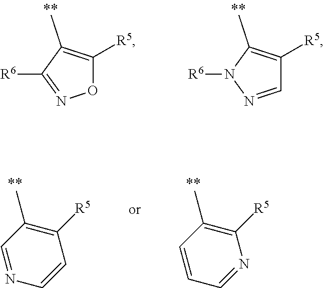

in which

** represents the point of attachment and

L represents CH₂

and the solvates, salts and solvates of the salts thereof.

Particular preference in the context of the present invention is given to compounds of the general formula (I) in which

R¹ represents hydrogen or methylamino,

R² represents hydrogen or methyl,

R^{3a}, R^{3b} represents hydrogen,

R⁴ represents methyl,

R⁵ and R⁶ independently of one another represent methyl, methoxy or hydrogen,

n represents the number 1 or 2,

X⁻ represents bromide, chloride or formate and

A represents a positively charged aza heteroaromatic of the formula

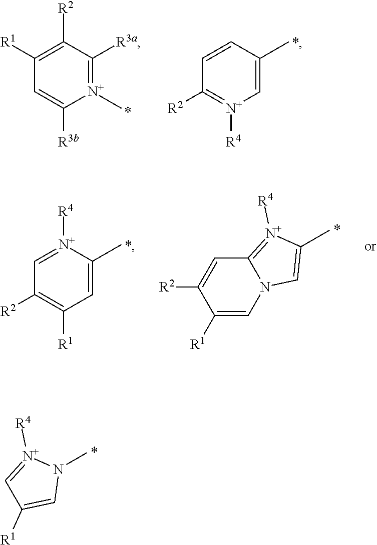

in which

* represents the point of attachment,

D represents a heteroaromatic of the formula

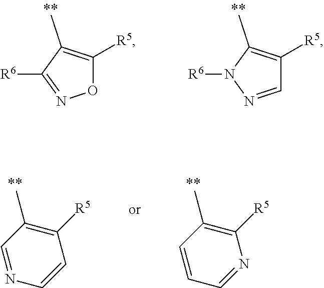

in which

** represents the point of attachment and

L represents CH₂,

and the solvates, salts and solvates of the salts thereof.

In the context of the present invention, preference is also given to compounds of the formula (I) selected from the group consisting of
1-[2-({[3-(3,5-dimethyl-1,2-oxazol-4-yl)imidazo[1,2-a]pyridin-7-yl]carbonyl}amino)ethyl]-4-(methylamino)pyridinium chloride hydrochloride

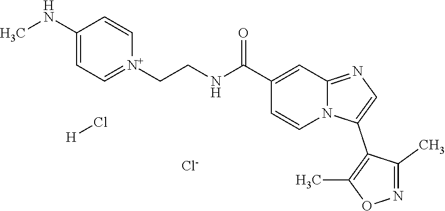

2-[({[3-(3,5-dimethyl-1,2-oxazol-4-yl)imidazo[1,2-a]pyridin-7-yl]carbonyl}amino)methyl]-1-methylimidazo[1,2-a]pyridin-1-ium formate

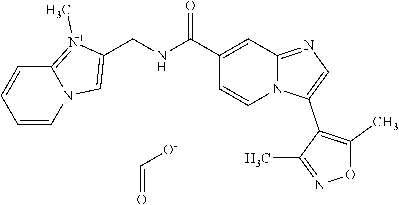

1-[2-({[3-(3,5-dimethyl-1,2-oxazol-4-yl)imidazo[1,2-a]pyridin-7-yl]carbonyl}amino)ethyl]-4-(methylamino)pyridinium formate

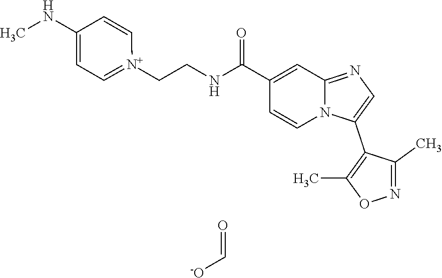

1-[2-({[3-(3,5-dimethyl-1,2-oxazol-4-yl)imidazo[1,2-a]pyridin-7-yl]carbonyl}amino)ethyl]-4-(methylamino)pyridinium chloride

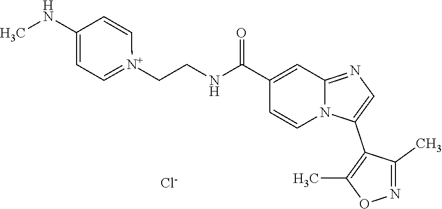

1-[2-({[3-(1,4-dimethyl-1H-pyrazol-5-yl)imidazo[1,2-a]pyridin-7-yl]carbonyl}amino)ethyl]-4-(methylamino)pyridinium formate

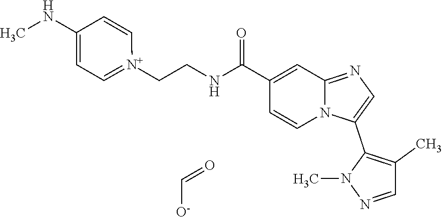

1-[2-({[3-(2-methoxypyridin-3-yl)imidazo[1,2-a]pyridin-7-yl]carbonyl}amino)ethyl]-4-(methylamino)pyridinium formate

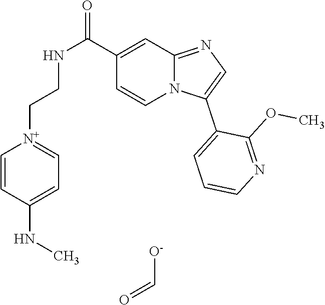

2-[({[3-(2-methoxypyridin-3-yl)imidazo[1,2-a]pyridin-7-yl]carbonyl}amino)methyl]-1-methylimidazo[1,2-a]pyridin-1-ium formate

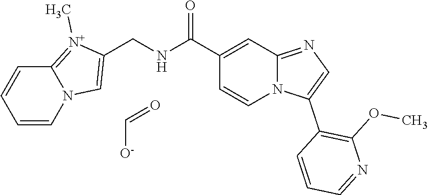

1-[2-({[3-(2-methoxypyridin-3-yl)imidazo[1,2-a]pyridin-7-yl]carbonyl}amino)ethyl]-3-methyl-4-(methylamino)pyridinium formate

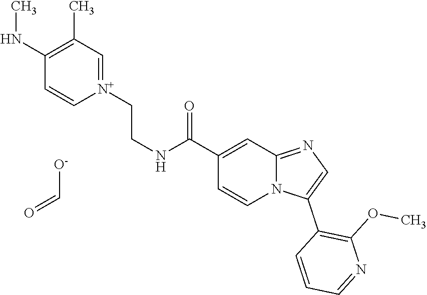

1-[2-({[3-(4-methoxypyridin-3-yl)imidazo[1,2-a]pyridin-7-yl]carbonyl}amino)ethyl]-4-(methylamino)pyridinium bromide

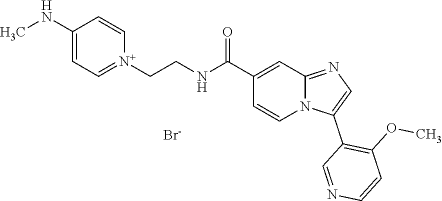

and the solvates, salts and solvates of the salts thereof.

The invention further provides a process for preparing the compounds of the formula (I) according to the invention, characterized in that

a compound of the formula (II) or its corresponding carboxylic acid

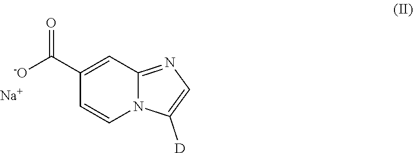

in which D has the meaning given above, is reacted in an inert solvent with a condensing agent such as, for example, 1-(3-dimethylaminopropyl)-3-ethylcarbodiimide hydrochloride in the presence of a base such as, for example, 4-dimethylaminopyridine with a compound of the formula (III)

A-(L)ₙ-NH₂ (III)

in which A, L and n have the meaning given above.

Inert solvents for the process step (II)+(III)→(I) are, for example, halohydrocarbons such as dichloromethane, trichloroethylene, chloroform or chlorobenzene, ethers such as diethyl ether, dioxane, tetrahydrofuran, glycol dimethyl ether or diethylene glycol dimethyl ether, hydrocarbons such as benzene, toluene, xylene, hexane, cyclohexane or mineral oil fractions, or other solvents such as acetonitrile, N,N-dimethylformamide, N,N-dimethylacetamide, dimethyl sulfoxide, N,N′-dimethylpropyleneurea (DMPU), N-methylpyrrolidone (NMP) or pyridine. It is equally possible to use mixtures of the solvents mentioned. Preference is given to using dichloromethane, tetrahydrofuran or pyridine. Particular preference is given to using dichloromethane.

Suitable for use as condensing agents for the amide formation in the process step (II)+(III)→(I) are, for example, carbodiimides such as N,N′-diethyl-, N,N′-dipropyl-, N,N′-diisopropyl-, N,N′-dicyclohexylcarbodiimide (DCC) or N-(3-dimethylaminopropyl)-N′-ethylcarbodiimide hydrochloride (EDC), phosgene derivatives such as N,N′-carbonyldiimidazole (CDI), 1,2-oxazolium compounds such as 2-ethyl-5-phenyl-1,2-oxazolium 3-sulfate or 2-tert-butyl-5-methylisoxazolium perchlorate, acylamino compounds such as 2-ethoxy-1-ethoxycarbonyl-1,2-dihydroquinoline or isobutyl chloroformate, propanephosphonic anhydride (T3P), 1-chloro-N,N,2-trimethylprop1-ene-1-amine, diethyl cyanophosphonate, bis(2-oxo-3-oxazolidinyl)phosphoryl chloride, benzotriazol-1-yloxytris(dimethylamino)phosphonium hexafluorophosphate, benzotriazol-1-yloxytris(pyrrolidino)phosphonium hexafluorophosphate (PyBOP), O-(benzotriazol-1-yl)-N,N,N′,N′-tetramethyluronium tetrafluoroborate (TBTU), O-(benzotriazol-1-yl)-N,N,N,N′-tetramethyluronium hexafluorophosphate (HBTU), 2-(2-oxo-1-(2H)-pyridyl)-1,1,3,3-tetramethyluronium tetrafluoroborate (TPTU), 0-(7-azabenzotriazol-1-yl)-N,N,N′,N′-tetramethyluronium hexafluorophosphate (HATU) or 0-(1H-6-chlorobenzotriazol-1-yl)-1,1,3,3-tetramethyluronium tetrafluoroborate (TCTU), optionally in combination with further auxiliaries such as 1-hydroxybenzotriazole (HOBt) or N-hydroxysuccinimide (HOSu). Preference is given to using EDC, HATU, DCC and T3P. Particular preference is given to using EDC.

Suitable for use as bases for the amide formation in process step (II)+(III)→(I) are, for example, alkali metal carbonates, for example sodium carbonate or potassium carbonate or sodium bicarbonate or potassium bicarbonate, or organic bases such as trialkylamines, for example triethylamine (TEA), N-methylmorpholine, N-methylpiperidine or N,N-diisopropylethylamine (DIPEA) or 4-(dimethylamino)pyridine (DMAP). Preference is given to using DMAP, TEA and DIPEA. Particular preference is given to using DMAP.

The condensation (II)+(III)→(I) is generally carried out in a temperature range of from −20° C. to +100° C., preferably at from 0° C. to +60° C. The conversion can be effected at standard, elevated or reduced pressure (for example from 0.5 to 5 bar). In general, room temperature and standard pressure are employed.

Alternatively, the carboxylate of the formula (II) can also first be converted to the corresponding carbonyl chloride and the latter can then be converted directly or in a separate reaction with a compound of the formula (III) into the compounds of the invention. The formation of carbonyl chlorides from carboxylic acids is carried out by the methods known to those skilled in the art, for example by treatment of (II) or the corresponding free carboxylic acid with thionyl chloride, sulfuryl chloride or oxalyl chloride, in the presence of a suitable base, for example in the presence of pyridine, and optionally with addition of dimethylformamide, optionally in a suitable inert solvent.

In the condensations (II)+(III)→(I), the nature of the work-up determines which counteranion X⁻ is obtained in the compounds according to the invention. If, for example, the crude product is purified by preparative HPLC using an aqueous mobile phase comprising formic acid, formates are obtained. If, on the other hand, the crude product is purified, for example, by column chromatography on amino-functionalized silica gel (from Biotage, SNAP NH-Cartridge), chlorides or bromides are obtained, depending on the synthesis route. If the alkylation of A^{1′} is carried out using phthalimide-protected chloroethylamine(IV)

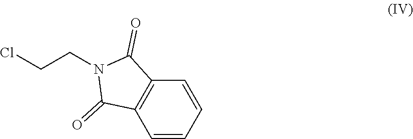 [SMILES: O=C1c2ccccc2C(=O)N1CCCl]

to give the building block (V)

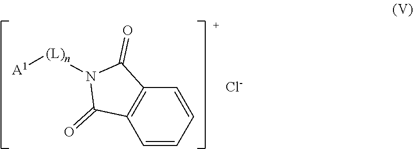

according to the reaction equation

A^{1′}+(IV)→(V)

and the subsequent deprotection is carried out using hydrochloric acid (also cf. Schema 1), chlorides are obtained.

If the alkylation is carried out using an appropriate bromide and the protective group is removed with hydrogen bromide, bromides are obtained. The other physiologically acceptable counteranions can be obtained from the formates, chlorides or bromides using ion exchangers.

The compounds employed are commercially available, known from the literature or can be prepared in analogy to literature processes.

The general process is illustrated in an exemplary manner by the scheme below (Scheme 1): Scheme 1:

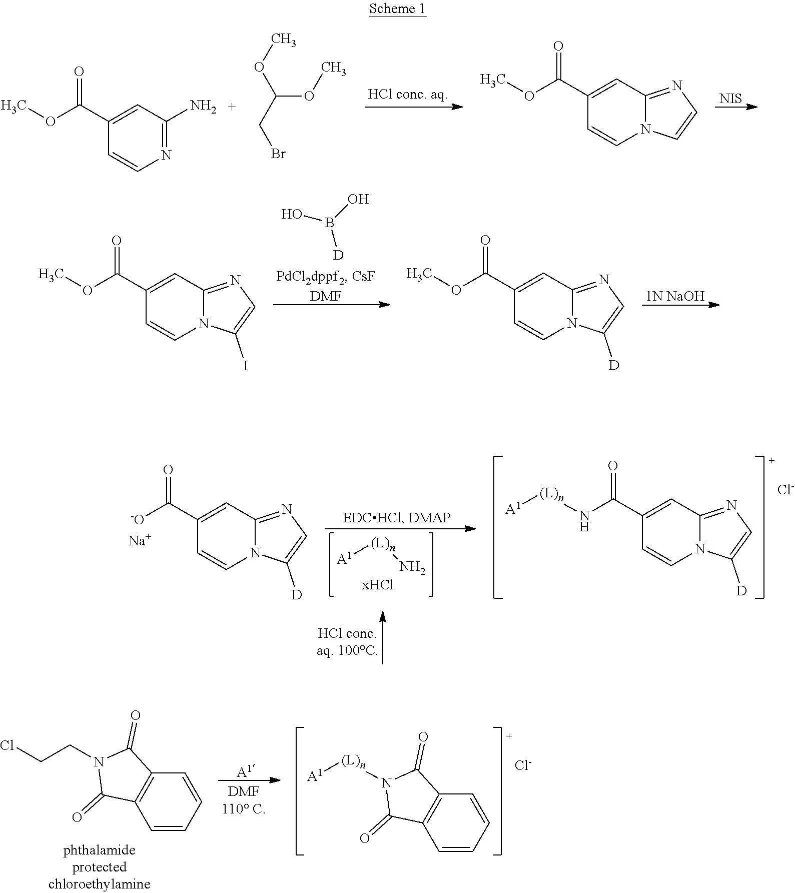

where A^{1′} represents

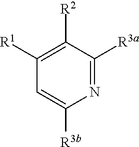

and A¹ represents

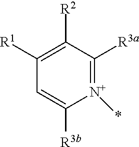

and *, L, n, D, R¹, R², R^{3a} and R^{3b} have the meaning indicated above.

A further general process is illustrated by way of example by the scheme below (Scheme 2):

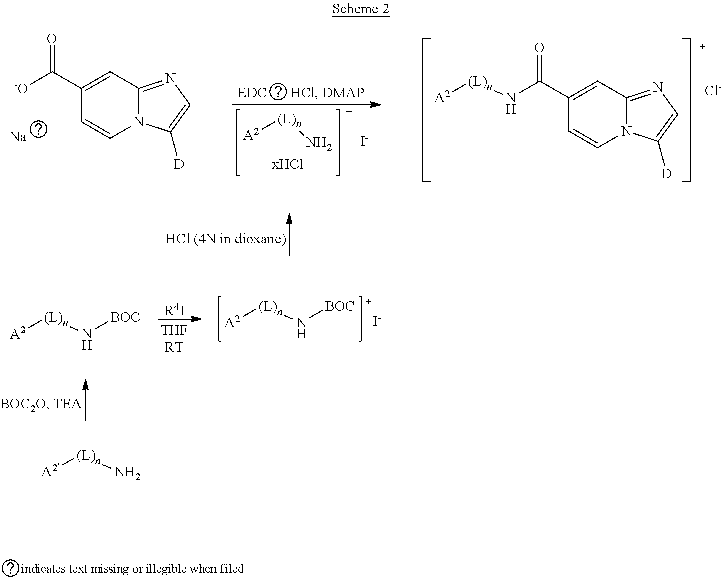

where A²^{₁} represents

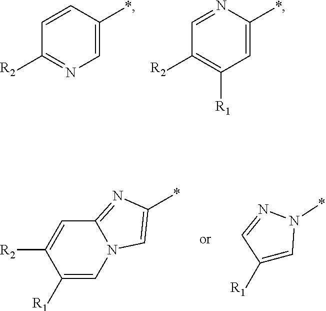

A² represents

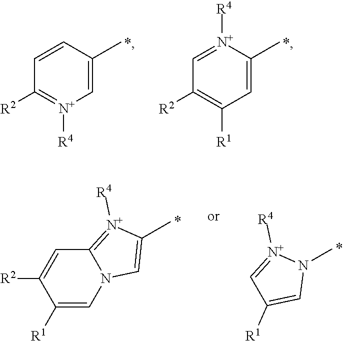

and*, L, n, D, R¹, R² and R⁴ have the meaning indicated above.

An alternative process variant is shown in Scheme 3:

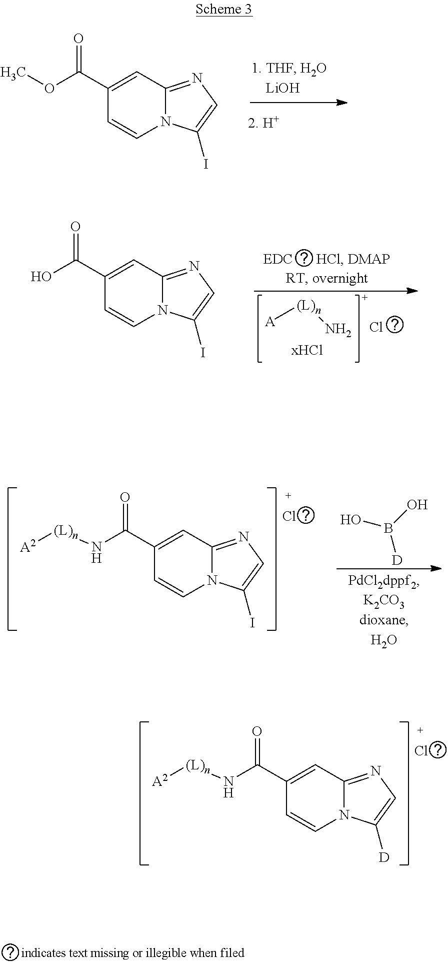

where A, L, n, and D have the meaning indicated above. The compound [A(L)ₙNH₂xHCl]⁺Cl⁻ was obtained as described above.

Detailed procedures can also be found in the Experimental Part, in the section on the preparation of the starting compounds and intermediates.

The compounds of the invention have valuable pharmacological properties and can be used for treatment and/or prophylaxis of disorders in humans and animals.

The compounds according to the invention are potent, chemically stable antagonists of the ADRA2B receptor and are therefore suitable for the treatment and/or prevention of disorders and pathological processes, in particular cardiovascular, nephrological, neurological and central nervous disorders.

In the context of the present invention, disorders of the cardiovascular system or cardiovascular disorders are understood to mean, for example, the following disorders: acute and chronic heart failure, arterial hypertension, coronary heart disease, stable and unstable angina pectoris, myocardial ischaemia, myocardial infarction, coronary microvascular dysfunction, microvascular obstruction, no-reflow phenomenon, shock, atherosclerosis, cardiac hypertrophy, cardiac fibrosis, atrial and ventricular arrhythmias, transitory and ischaemic attacks, stroke, ischaemic and haemorrhagic stroke, pre-eclampsia, inflammatory cardiovascular disorders, peripheral and cardiac vascular disorders, peripheral perfusion disorders, peripheral arterial occlusive disease, primary and secondary Raynaud's syndrome, impaired microcirculation, arterial pulmonary hypertension, spasms of the coronary arteries and peripheral arteries, thromboses, thromboembolic disorders, oedema development, for example pulmonary oedema, cerebral oedema, renal oedema or heart failure-related oedema, and restenoses such as after thrombolysis treatments, percutaneous transluminal angioplasty (PTA), transluminal coronary angioplasty (PTCA), heart transplants and bypass operations, and micro- and macrovascular damage (vasculitis), reperfusion damage, arterial and venous thromboses, microalbuminuria, myocardial insufficiency, endothelial dysfunction, peripheral and cardiac vascular disorders.

In the context of the present invention, the term “heart failure” also includes more specific or related types of disease, such as acutely decompensated heart failure, right heart failure, left heart failure, global failure, ischaemic cardiomyopathy, dilated cardiomyopathy, congenital heart defects, heart valve defects, heart failure associated with heart valve defects, mitral valve stenosis, mitral valve insufficiency, aortic valve stenosis, aortic valve insufficiency, tricuspid stenosis, tricuspid insufficiency, pulmonary valve stenosis, pulmonary valve insufficiency, combined heart valve defects, myocardial inflammation (myocarditis), chronic myocarditis, acute myocarditis, viral myocarditis, diabetic heart failure, alcoholic cardiomyopathy, cardiac storage disorders, heart failure with preserved ejection fraction (HFpEF), diastolic heart failure and heart failure with reduced ejection fraction (HFrEF systolic heart failure).

In the context of the present invention, the term atrial and ventricular arrhythmias also includes more specific or related types of disease, such as: atrial fibrillation, paroxysmal atrial fibrillation, intermittierent atrial fibrillation, permanent atrial fibrillation, atrial flutter, sinusoidal arrhythmia, sinusoidal tachycardia, passive heterotopia, active heterotopia, escape systoles, extrasystoles, impulse conduction disorders, sick sinus syndrome, hypersensitive carotid sinus, tachycardias, AV node reentry tachycardia, atriventricular reentry tachycardia, WPW syndrome (Wolff-Parkinson-White), Mahaim tachycardia, hidden accessory conduction pathway, permanent junctional reentry tachycardia, focal atrial tachycardia, junctional ectopic tachycardia, atrial reentry tachycardia, ventricular tachycardia, ventricular flutter, ventricular fibrillation, sudden cardiac death.

In the context of the present invention, the term coronary heart disease also encompasses more specific or related types of disease, such as: ischaemic heart disease, stable angina pectoris, acute coronary syndrome, unstable angina pectoris, NSTEMI (non-ST elevation myocardial infarction), STEMI (ST elevation myocardial infarction), ischaemic heart muscle damage, heart rhythm dysfunctions and myocardial infarction.

In the context of the present invention, disorders of the central nervous and neurological system or central nervous and neurological disorders are to be understood as meaning, for example, the following disorders: transitory and ischaemic attacks, stroke, ischaemic and haemorrhagic stroke, depression, anxiety disorders, posttraumatic stress disorder, polyneuropathy, diabetic polyneuropathy, stress-related hypertension.

The compounds according to the invention are further suitable for the prophylaxis and/or treatment of polycystic kidney disease (PCKD) and of the syndrome of inappropriate ADH secretion (SIADH).

Furthermore, the compounds according to the invention are suitable for the treatment and/or prophylaxis of kidney disorders, in particular of acute and chronic kidney insufficiency and acute and chronic renal failure.

For the purpose of the present invention, the term acute renal insufficiency encompasses acute manifestations of kidney disease, of kidney failure and/or renal insufficiency with and without the need for dialysis, and also underlying or related renal disorders such as renal hypoperfusion, intradialytic hypotension, volume deficiency (e.g. dehydration, blood loss), shock, acute glomerulonephritis, haemolytic-uraemic syndrome (HUS), vascular catastrophe (arterial or venous thrombosis or embolism), cholesterol embolism, acute Bence-Jones kidney in the event of plasmacytoma, acute supravesicular or subvesicular efflux obstructions, immunological renal disorders such as kidney transplant rejection, immune complex-induced renal disorders, tubular dilatation, hyperphosphataemia and/or acute renal disorders which can be characterized by the need for dialysis, and also in the case of partial resections of the kidney, dehydration through forced diuresis, uncontrolled blood pressure rise with malignant hypertension, urinary tract obstruction and infection and amyloidosis, and systemic disorders with glomerular factors, such as rheumatological-immunological systemic disorders, for example lupus erythematodes, renal artery thrombosis, renal vein thrombosis, analgesic nephropathy and renal tubular acidosis, and X-ray contrast agent- and medicament-induced acute interstitial renal disorders.

In the context of the present invention, the term “chronic renal insufficiency” encompasses chronic manifestations of kidney disease, of kidney failure and/or renal insufficiency with and without the need for dialysis, and also underlying or related renal disorders such as renal hypoperfusion, intradialytic hypotension, obstructive uropathy, glomerulopathy, glomerular and tubular proteinuria, renal oedema, haematuria, primary, secondary and chronic glomerulonephritis, membranous and membranoproliferative glomerulonephritis, Alport syndrome, glomerulosclerosis, tubulointerstitial disorders, nephropathic disorders such as primary and congenital kidney disease, renal inflammation, immunological renal disorders such as kidney transplant rejection, immune complex-induced renal disorders, diabetic and non-diabetic nephropathy, pyelonephritis, renal cysts, nephrosclerosis, hypertensive nephrosclerosis and nephrotic syndrome, which can be characterized diagnostically, for example, by abnormally reduced creatinine and/or water excretion, abnormally elevated blood concentrations of urea, nitrogen, potassium and/or creatinine, altered activity of renal enzymes, for example glutamyl synthetase, altered urine osmolarity or urine volume, elevated microalbuminuria, macroalbuminuria, glomerular and arteriolar lesions, tubular dilatation, hyperphosphataemia and/or the need for dialysis, and also for renal cell carcinomas, after partial resections of the kidney, dehydration through forced diuresis, uncontrolled blood pressure increase with malignant hypertension, urinary tract obstruction and infection and amyloidosis and systemic disorders with glomerular factors, such as rheumatological-immunological systemic disorders, for example lupus erythematodes, and renal artery stenosis, renal artery thrombosis, renal vein thrombosis, analgesic nephropathy and renal-tubular acidosis. In addition, X-ray contrast agent- and medicament-induced chronic interstitial renal disorders, metabolic syndrome and dyslipidaemia. The present invention also encompasses the use of the compounds according to the invention for treatment and/or prophylaxis of sequelae of renal insufficiency, for example pulmonary oedema, heart failure, uraemia, anaemia, electrolyte disorders (for example hyperkalaemia, hyponatraemia) and disorders in bone and carbohydrate metabolism.

In addition, the compounds according to the invention are also suitable for treatment and/or prophylaxis of pulmonary arterial hypertension (PAH) and other forms of pulmonary hypertension (PH), of chronic obstructive pulmonary disease (COPD), of acute respiratory distress syndrome (ARDS), of acute lung injury (ALI), of alpha-1-antitrypsin deficiency (AATD), of pulmonary fibrosis, of pulmonary emphysema (for example pulmonary emphysema caused by cigarette smoke), of cystic fibrosis (CF), of acute coronary syndrome (ACS), heart muscle inflammations (myocarditis) and other autoimmune cardiac disorders (pericarditis, endocarditis, valvolitis, aortitis, cardiomyopathies), cardiogenic shock, aneurysms, sepsis (SIRS), multiple organ failure (MODS, MOF), inflammatory disorders of the kidney, chronic intestinal disorders (IBD, Crohn's Disease, UC), pancreatitis, peritonitis, rheumatoid disorders, inflammatory skin disorders and inflammatory eye disorders.

The compounds according to the invention can also be used for treatment and/or prophylaxis of asthmatic disorders of varying severity with intermittent or persistent characteristics (refractive asthma, bronchial asthma, allergic asthma, intrinsic asthma, extrinsic asthma, medicament- or dust-induced asthma), of various forms of bronchitis (chronic bronchitis, infectious bronchitis, eosinophilic bronchitis), of Bronchiolitis obliterans, bronchiectasis, pneumonia, idiopathic interstitial pneumonia, farmer's lung and related diseases, of coughs and colds (chronic inflammatory cough, iatrogenic cough), inflammation of the nasal mucosa (including medicament-related rhinitis, vasomotoric rhinitis and seasonal allergic rhinitis, for example hay fever) and of polyps.

The compounds according to the invention are also suitable for treatment and/or prophylaxis of fibrotic disorders of the internal organs, for example the lung, the heart, the kidney, the bone marrow and in particular the liver, and also dermatological fibroses and fibrotic eye disorders. In the context of the present invention, the term “fibrotic disorders” encompasses particularly the following terms: hepatic fibrosis, cirrhosis of the liver, pulmonary fibrosis, endomyocardial fibrosis, cardiomyopathy, nephropathy, glomerulonephritis, interstitial renal fibrosis, fibrotic damage resulting from diabetes, bone marrow fibrosis and similar fibrotic disorders, scleroderma, morphea, keloids, hypertrophic scarring (also following surgical procedures), nevi, diabetic retinopathy and proliferative vitroretinopathy.

In addition, the compounds according to the invention can also be used for treatment and/or prophylaxis of dyslipidemias (hypercholesterolaemia, hypertriglyceridaemia, elevated concentrations of the postprandial plasma triglycerides, hypoalphalipoproteinaemia, combined hyperlipidaemias), metabolic disorders (type 1 and type 2 diabetes, metabolic syndrome, overweight, obesity), nephropathy and neuropathy), cancers (skin cancer, brain tumors, breast cancer, bone marrow tumors, leukaemias, liposarcomas, carcinoma of the gastrointestinal tract, of the liver, pancreas, lung, kidney, urinary tract, prostate and genital tract, and also malignant tumors in the lymphoproliferative system, for example Hodgkin's and non-Hodgkin's lymphoma), of disorders of the gastrointestinal tract and of the abdomen (glossitis, gingivitis, periodontitis, esophagitis, eosinophilic gastroenteritis, mastocytosis, Crohn's disease, colitis, proctitis, pruritus ani, diarrhoea, coeliac disease, hepatitis, chronic hepatitis, hepatic fibrosis, cirrhosis of the liver, pancreatitis and cholecystitis), skin disorders (allergic skin disorders, psoriasis, acne, eczema, neurodermitis, various forms of dermatitis, and also keratitis, bullosis, vasculitis, cellulitis, panniculitis, lupus erythematodes, erythema, lymphoma, skin cancer, Sweet's syndrome, Weber-Christian syndrome, scarring, warts, chillblains), of disorders of the skeletal bone and of the joints, and also of the skeletal muscle (various forms of arthritis, various forms of arthropathies, scleroderma and of further disorders with an inflammatory or immunological component, for example paraneoplastic syndrome, in the event of rejection reactions after organ transplants and for wound healing and angiogenesis, especially in the case of chronic wounds.

The compounds of the formula (I) according to the invention are additionally suitable for treatment and/or prophylaxis of ophthalmologic disorders, for example glaucoma, normotensive glaucoma, high intraocular pressure and combinations thereof, of age-related macular degeneration (AMD), of dry or non-exudative AMD, moist or exudative or neovascular AMD, choroidal neovascularization (CNV), detached retina, diabetic retinopathy, atrophic lesions to the retinal pigment epithelium (RPE), hypertrophic lesions to the retinal pigment epithelium (RPE), diabetic macular edema, diabetic retinopathy, retinal vein occlusion, choroidal retinal vein occlusion, macular edema, macular edema due to retinal vein occlusion, angiogenesis at the front of the eye, for example corneal angiogenesis, for example following keratitis, cornea transplant or keratoplasty, corneal angiogenesis due to hypoxia (extensive wearing of contact lenses), pterygium conjunctiva, subretinal edema and intraretinal edema.

In addition, the compounds of the formula (I) according to the invention are suitable for treatment and/or prophylaxis of elevated and high intraocular pressure resulting from traumatic hyphema, periorbital edema, postoperative viscoelastic retention, intraocular inflammation, use of corticosteroids, pupillary block or idiopathic causes, and of elevated intraocular pressure following trabeculectomy and due to pre-operative additions.

By virtue of their biochemical and pharmacological property profile, the compounds according to the invention are particularly suitable for the treatment and/or prophylaxis of acute heart failure, right heart failure, left heart failure, global failure, diabetic heart failure, heart failure with preserved ejection fraction (HFpEF), diastolic heart failure, heart failure with reduced ejection fraction (HFrEF systolic heart failure), coronary heart disease, stable and unstable angina pectoris, myocardial ischaemia, acute coronary syndrome, NSTEMI (non-ST elevation myocardial infarction), STEMI (ST elevation myocardial infarction), ischaemic heart muscle damage, myocardial infarction, coronary microvascular dysfunction, microvascular obstruction, no-reflow phenomenon, transitory and ischaemic attacks, ischaemic and haemorrhagic stroke, peripheral and cardial vascular disorders, impaired peripheral circulation, peripheral arterial occlusive disease, primary and secondary Raynaud's syndrome, impaired microcirculation, arterial pulmonary hypertension, spasms of coronary arteries and peripheral arteries, restenoses such as after thrombolysis therapy, percutaneous transluminal angioplasty (PTA), transluminal coronary angioplasty (PTCA), reperfusion damage, endothelial dysfunction, ischaemic cardiomyopathy, renal insufficiency and nephropathies and stress-related hypertension.

The aforementioned well-characterized diseases in humans can also occur with comparable etiology in other mammals and can likewise be treated therein with the compounds of the present invention.

The present invention further provides the compounds according to the invention for use in a method for the treatment and/or prophylaxis of acute heart failure, coronary heart disease, myocardial infarction, microvascular dysfunction, peripheral arterial occlusive disease, renal insufficiency and nephropathies.

The treatment or prevention of a disease, a condition, a disorder, an injury or a health problem may be partial or complete.

The present invention thus further provides for the use of the compounds of the invention for treatment and/or prevention of disorders, especially of the aforementioned disorders.

The present invention further provides for the use of the compounds of the invention for production of a medicament for treatment and/or prevention of disorders, especially of the aforementioned disorders.

The present invention further provides a medicament comprising at least one of the compounds of the invention for treatment and/or prevention of disorders, especially of the aforementioned disorders.

The present invention further provides for the use of the compounds of the invention in a method for treatment and/or prevention of disorders, especially of the aforementioned disorders.

The present invention further provides a method of treatment and/or prevention of disorders, especially of the aforementioned disorders, using an effective amount of at least one of the compounds of the invention.

The compounds of the invention can be used alone or, if required, in combination with one or more other pharmacologically active substances, provided that this combination does not lead to undesirable and unacceptable side effects. The present invention therefore further provides medicaments comprising at least one of the compounds of the invention and one or more further drugs, especially for treatment and/or prevention of the aforementioned disorders. Preferred examples of combination active ingredients suitable for this purpose include:
hypotensive drugs, by way of example and with preference from the group of calcium antagonists, angiotensin AII antagonists, ACE inhibitors, NEP inhibitors, vasopeptidase inhibitors, and combinations of these such as sacubitril/valsartan, furthermore nicorandil, endothelin antagonists, thromboxan A2 antagonists, renin inhibitors, alpha-receptor blockers, beta-receptor blockers, mineralocorticoid receptor antagonists, rho kinase inhibitors, diuretics and also other vasoactive compounds such as adenosine and adenosine receptor agonists.
antiarrhythmics, by way of example and with preference sodium channel blockers, beta receptor blockers, potassium channel blockers, calcium antagonists, I_{f} channel blockers, digitalis, parasympatholytics (vagolytics), sympathomimetics and other antiarrhythmics such as adenosine, adenosine receptor agonists and vernakalant;
compounds having a positive inotropic effect, for example cardiac glycosides (digoxin), beta-adrenergic and dopaminergic agonists such as isoprenaline, adrenaline, noradrenaline, dopamine or dobutamine and serelaxin;
vasopressin receptor antagonists, by way of example and with preference conivaptan, tolvaptan, lixivaptan, mozavaptan, satavaptan, SR-121463, RWJ 676070 or BAY 86-8050, and also the compounds described in WO 2010/105770, WO2011/104322 and WO 2016/071212;
natriuretic peptides, by way of example and with preference atrial natriuretic peptide (ANP), natriuretic peptide type B (BNP, nesiritide) natriuretic peptide type C (CNP) or urodilatin;
activators of cardial myosin, by way of example and with preference omecamtiv mecarbil (CK-1827452);
calcium sensitizers, a preferred example being levosimendan
active compounds which affect mitochondria function/ROS production, for example Bendavia/elamipritide
compounds which modulate the energy metabolism of the heart, by way of example and with preference etomoxir, dichloroacetate, ranolazine or trimetazidine, full or partial adenosine A1 receptor agonists such as GS-9667 (formerly known as CVT-3619), capadenoson and neladenoson;
compounds which modulate the heart rate, for example ivabradine
compounds which inhibit the degradation of cyclic guanosine monophosphate (cGMP) and/or cyclic adenosine monophosphate (cAMP), for example inhibitors of phosphodiesterases (PDE) 1, 2, 3, 4 and/or 5, especially PDE 5 inhibitors such as sildenafil, vardenafil and tadalafil, udenafil, desantafil, avanafil, mirodenafil, lodenafil or PF-00489791;
antithrombotic agents, by way of example and with preference from the group of the platelet aggregation inhibitors, the anticoagulants or the profibrinolytic substances;
bronchodilatory agents, byway of example and with preference from the group of the beta-adrenergic receptor agonists, such as especially albuterol, isoproterenol, metaproterenol, terbutalin, formoterol or salmeterol, or from the group of the anticholinergics, such as especially ipratropium bromide;
anti-inflammatory agents, by way of example and with preference from the group of the glucocorticoids, such as especially prednisone, prednisolone, methylprednisolone, triamcinolone, dexamethasone, beclomethasone, betamethasone, flunisolide, budesonide or fluticasone and also non-steroidal anti-inflammatory drugs (NSAIDs) such as, in particular, acetylsalicylic acid (aspirin), ibuprofen and naproxen, 5-aminosalicylic acid derivatives, leukotriene antagonists, TNF-alpha inhibitors and chemokine receptor antagonists such as CCR1, 2 and/or 5 inhibitors;
active compounds which modulate lipid metabolism, by way of example and with preference from the group of the thyroid receptor agonists, cholesterol synthesis inhibitors such as, by way of example and preferably, HMG-CoA reductase inhibitors or squalene synthesis inhibitors, the ACAT inhibitors, CETP inhibitors, MTP inhibitors, PPAR-alpha, PPAR-gamma and/or PPAR-6 agonists, cholesterol absorption inhibitors, lipase inhibitors, polymeric bile acid adsorbents, bile acid reabsorption inhibitors and lipoprotein(a) antagonists.
compounds which inhibit the signal transduction cascade, by way of example and with preference from the group of the kinase inhibitors, especially from the group of the tyrosine kinase and/or serine/threonine kinase inhibitors;
compounds which inhibit the degradation and alteration of the extracellular matrix, by way of example and with preference inhibitors of the matrix metalloproteases (MMPs), especially inhibitors of chymase, stromelysin, collagenases, gelatinases and aggrecanases (in this context particularly of MMP-1, MMP-3, MMP-8, MMP-9, MMP-10, MMP-11 and MMP-13) and of metalloelastase (MMP-12) and neutrophile elastase (HNE), such as sivelestat or DX-890;
compounds which block the binding of serotonin to its receptor, by way of example and with preference antagonists of the 5-HT_{2b} receptor;
organic nitrates and NO donors, for example sodium nitroprusside, nitroglycerin, isosorbide mononitrate, isosorbide dinitrate, molsidomine or SIN-1, and inhaled NO;
NO-independent but haem-dependent stimulators of soluble guanylate cyclase, such as especially the compounds described in WO 00/06569, WO 02/42301, WO 03/095451, WO 2011/147809, WO2014/068099 and 2014/131760;
NO- and haem-independent activators of soluble guanylate cyclase, such as especially the compounds described in WO 01/19355, WO 01/19780, WO2012/139888 and 2014/012934;
compounds which increase the synthesis of cGMP, for example sGC modulators such as, by way of example and with preference, riociguat, cinaciguat or vericiguat;
prostacyclin analogues, by way of example and with preference iloprost, beraprost, treprostinil or epoprostenol;
compounds which inhibit soluble epoxide hydrolase (sEH), for example N,N′-dicyclohexylurea, 12-(3-adamantan-1-ylureido)dodecanoic acid or 1-adamantan-1-yl-3-{5-[2-(2-ethoxyethoxy)ethoxy]pentyl}urea;
active compounds which modulate glucose metabolism, for example insulins, biguanides, thiazolidinediones, sulfonylureas, acarbose, DPP4 inhibitors, GLP-1 analogs or SGLT-1 inhibitors;
active compounds which modulate neurotransmitters, for example tricyclic antidepressants such as amitryptiline and imipramine, monooxidase (MAO) inhibitors such as moclobemid, serotonine/noradrenaline reuptake inhibitors such as venlaflaxin, selective serotonine reuptake inhibitors such as sertraline or noradrenaline/serotonin-selective antidepressants such as mirtazepine.
anxiolytic, sedative and hypnotically acting substances, so-called tranquilizers, such as benzodiazepines with short or medium-term action.
painkillers such as opiates.

Hypotensive agents are preferably understood to mean compounds from the group of calcium antagonists, angiotensin AII antagonists, ACE inhibitors, endothelin antagonists, TXA2 antagonists, renin inhibitors, alpha-receptor blockers, beta-receptor blockers, mineralocorticoid receptor antagonists, rho kinase inhibitors, and the diuretics.

In a preferred embodiment of the invention, the compounds of the invention are administered in combination with a calcium antagonist, by way of example and with preference nifedipine, amlodipine, verapamil or diltiazem.

In a preferred embodiment of the invention, the compounds according to the invention are administered in combination with an angiotensin AII antagonist, by way of example and with preference losartan, candesartan, valsartan, telmisartan or embursatan, irbesartan, olmesartan, eprosartan or azilsartan or a dual angiotensin AII antagonist/NEP inhibitor, for example and with preference Entresto (LCZ696, valsartan/sacubitril).

In a preferred embodiment of the invention, the compounds according to the invention are administered in combination with an ACE inhibitor, by way of example and with preference enalapril, captopril, lisinopril, ramipril, delapril, fosinopril, quinopril, perindopril or trandopril.

In a preferred embodiment of the invention, the compounds according to the invention are administered in combination with an endothelin antagonist, by way of example and with preference bosentan, darusentan, ambrisentan, avosentan, macitentan, atrasentan or sitaxsentan.

In a preferred embodiment of the invention, the compounds according to the invention are used in combination with a thromboxane A2 antagonist, by way of example and with preference seratrodast or KP-496.

In a preferred embodiment of the invention, the compounds of the invention are administered in combination with a renin inhibitor, by way of example and with preference aliskiren, SPP-600 or SPP-800.

In a preferred embodiment of the invention, the compounds of the invention are administered in combination with an alpha-1 receptor blocker, by way of example and with preference prazosin.

In a preferred embodiment of the invention, the compounds of the invention are administered in combination with a beta receptor blocker, by way of example and with preference propranolol, atenolol, timolol, pindolol, alprenolol, oxprenolol, penbutolol, bupranolol, metipranolol, nadolol, mepindolol, carazalol, sotalol, metoprolol, betaxolol, celiprolol, bisoprolol, carteolol, esmolol, labetalol, carvedilol, adaprolol, landiolol, nebivolol, epanolol or bucindolol.

In a preferred embodiment of the invention, the compounds of the invention are administered in combination with a mineralocorticoid receptor antagonist, by way of example and with preference spironolactone, eplerenone or finerenone.

In a preferred embodiment of the invention, the compounds according to the invention are administered in combination with a rho kinase inhibitor, by way of example and with preference fasudil, Y-27632, SLx-2119, BF-66851, BF-66852, BF-66853, KI-23095, SB-772077, GSK-269962A or BA-1049.

In a preferred embodiment of the invention, the compounds of the invention are administered in combination with a diuretic, for example furosemide, torasemide, bumetanide and piretanide, with potassium-sparing diuretics, for example amiloride and triamterene, and also thiazide diuretics, for example hydrochlorothiazide, chlorthalidone, xipamide and indapamide.

Antithrombotic agents are preferably understood to mean compounds from the group of the platelet aggregation inhibitors, the anticoagulants or the profibrinolytic substances.

In a preferred embodiment of the invention, the compounds according to the invention are administered in combination with platelet aggregation inhibitors, by way of example and with preference aspirin, clopidogrel, prasugrel, ticlopidine, ticagrelor, cangrelor, elinogrel, tirofiban, PAR-1 antagonists such as, for example, vorapaxar, PAR-4 antagonists, EP3 antagonists such as, for example, DG041, or adenosine transporter inhibitors such as dipyridamol.

In a preferred embodiment of the invention, the compounds of the invention are administered in combination with a GPIIb/IIIa antagonist, by way of example and with preference tirofiban or abciximab.

In a preferred embodiment of the invention, the compounds according to the invention are administered in combination with a thrombin inhibitor, by way of example and with preference dabigatran, ximelagatran, melagatran, bivalirudin or clexane.

In a preferred embodiment of the invention, the compounds according to the invention are administered in combination with a factor Xa inhibitor, by way of example and with preference rivaroxaban, apixaban, edoxaban (DU-176b), darexaban, betrixaban, otamixaban, letaxaban, fidexaban, razaxaban, fondaparinux, idraparinux, and also thrombin inhibitors such as, by way of example and with preference dabigatran, dual thrombin/factor Xa inhibitors such as, by way of example and with preference tanogitran, or factor XIa inhibitors.

In a preferred embodiment of the invention, the compounds according to the invention are administered in combination with heparin or a low molecular weight (LMW) heparin derivative such as, for example, tinzaparin, certoparin, parnaparin, nadroparin, ardeparin, enoxaparin, reviparin, dalteparin, danaparoid, semuloparin (AVE 5026), adomiparin (M118) and EP-42675/ORG42675.

In a preferred embodiment of the invention, the compounds of the invention are administered in combination with a vitamin K antagonist, by way of example and with preference coumarins such as Macumar or phenprocoumon.

In a preferred embodiment of the invention, the compounds of the invention are administered in combination with profibrinolytic compounds, by way of example and with preference streptokinase, urokinase or plasminogen activator.

Lipid metabolism modifiers are preferably understood to mean compounds from the group of the CETP inhibitors, thyroid receptor agonists, cholesterol synthesis inhibitors such as HMG-CoA reductase inhibitors or squalene synthesis inhibitors, the ACAT inhibitors, MTP inhibitors, PPAR-alpha, PPAR-gamma and/or PPAR-6 agonists, cholesterol absorption inhibitors, polymeric bile acid adsorbents, bile acid reabsorption inhibitors, lipase inhibitors and the lipoprotein(a) antagonists.

In a preferred embodiment of the invention, the compounds of the invention are administered in combination with a CETP inhibitor, by way of example and with preference torcetrapib (CP-529 414), anacetrapib, JJT-705 or CETP vaccine (Avant).

In a preferred embodiment of the invention, the compounds of the invention are administered in combination with a thyroid receptor agonist, by way of example and with preference D-thyroxine, 3,5,3′-triiodothyronine (T3), CGS 23425 or axitirome (CGS 26214).

In a preferred embodiment of the invention, the compounds of the invention are administered in combination with an HMG-CoA reductase inhibitor from the class of statins, by way of example and with preference lovastatin, simvastatin, pravastatin, fluvastatin, atorvastatin, rosuvastatin or pitavastatin.

In a preferred embodiment of the invention, the compounds of the invention are administered in combination with a squalene synthesis inhibitor, by way of example and with preference BMS-188494 or TAK-475.

In a preferred embodiment of the invention, the compounds of the invention are administered in combination with an ACAT inhibitor, by way of example and with preference avasimibe, melinamide, pactimibe, eflucimibe or SMP-797.

In a preferred embodiment of the invention, the compounds of the invention are administered in combination with an MTP inhibitor, by way of example and with preference implitapide, BMS-201038, R-103757 or JTT-130.

In a preferred embodiment of the invention, the compounds of the invention are administered in combination with a PPAR-gamma agonist, by way of example and with preference pioglitazone or rosiglitazone.

In a preferred embodiment of the invention, the compounds according to the invention are administered in combination with a PPAR-6 agonist, by way of example and with preference GW 501516 or BAY 68-5042.

In a preferred embodiment of the invention, the compounds of the invention are administered in combination with a cholesterol absorption inhibitor, by way of example and with preference ezetimibe, tiqueside or pamaqueside.

In a preferred embodiment of the invention, the compounds of the invention are administered in combination with a lipase inhibitor, by way of example and with preference orlistat.

In a preferred embodiment of the invention, the compounds of the invention are administered in combination with a polymeric bile acid adsorber, by way of example and with preference cholestyramine, colestipol, colesolvam, CholestaGel or colestimide.

In a preferred embodiment of the invention, the compounds of the invention are administered in combination with a bile acid reabsorption inhibitor, by way of example and with preference ASBT(=IBAT) inhibitors, for example AZD-7806, S-8921, AK-105, BARI-1741, SC-435 or SC-635.

In a preferred embodiment of the invention, the compounds of the invention are administered in combination with a lipoprotein(a) antagonist, by way of example and with preference gemcabene calcium (CI-1027) or nicotinic acid.

Active compounds which inhibit signal transduction cascades are preferably understood to mean compounds from the group of the tyrosine kinase inhibitors and/or serine/threonine kinase inhibitors.

In a preferred embodiment of the invention, the compounds according to the invention are used in combination with a kinase inhibitor, by way of example and with preference bortezomib, canertinib, erlotinib, gefitinib, imatinib, lapatinib, lestaurtinib, lonafarnib, nintedanib, dasatinib, nilotinib, bosutinib, axitinib, telatinib, imatinib, brivanib, pazopanib, pegaptinib, pelitinib, semaxanib, sorafenib, regorafenib, sunitinib, tandutinib, tipifarnib, vatalanib, fasudil, lonidamine, leflunomide, BMS-3354825 or Y-27632.

Active compounds which modulate glucose metabolism are preferably understood to mean compounds from the group of the insulins, a sulfonylurea, acarbose, DPP4 inhibitors, GLP-1 analogues or SGLT-1 inhibitors.

Active compounds which modulate neurotransmitters are preferably understood to mean compounds from the group of the tricyclic antidepressants, monoamine oxidase (MAO) inhibitors, serotonin/noradrenaline reuptake inhibitors (SNR) and noradrenaline/serotonin-selective antidepressants (NaSSa).

In a preferred embodiment of the invention, the compounds of the invention are administered in combination with a tricyclic antidepressant, by way of example and with preference amitryptiline or imipramine.

In a preferred embodiment of the invention, the compounds of the invention are administered in combination with a monoamine oxidase (MAO) inhibitor, by way of example and with preference mocolobemide.

In a preferred embodiment of the invention, the compounds of the invention are administered in combination with a selective serotonin/noradrenaline reuptake inhibitor (SNRI), by way of example and with preference venlafaxine.

In a preferred embodiment of the invention, the compounds of the invention are administered in combination with a selective serotonin reuptake inhibitor such as sertraline.

In a preferred embodiment of the invention, the compounds according to the invention are administered in combination with a noradrenaline/serotonin-selective antidepressant (NaSSa), by way of example and with preference mirtazepine.

Active compounds having analgesic, anxiolytic or sedating properties are preferably understood to mean compounds from the group of the opiates and benzodiazepines.

In a preferred embodiment of the invention, the compounds of the invention are administered in combination with an opiate, by way of example and with preference morphine or sufentanil or fentanyl.

In a preferred embodiment of the invention, the compounds of the invention are administered in combination with a benzodiazepine, by way of example and with preference midazolam or diazepam.

Active compounds which increase the synthesis of cGMP such as, for example, sGC modulators, are preferably understood to mean compounds which stimulate or activate soluble guanylate cyclase.

In a preferred embodiment of the invention, the compounds according to the invention are administered in combination with sGC modulators, by way of example and with preference riociguat, cinaciguat or vericiguat.

In a preferred embodiment of the invention, the compounds of the invention are administered in combination with full or partial adenosine A1 receptor agonists such as GS-9667 (formerly known as CVT-3619), capadenoson and neladenoson or active compounds affecting mitochondrial function/ROS production, such as, for example, Bendavia/elamipritide.

In a preferred embodiment of the invention, the compounds according to the invention are administered in combination with a TGFbeta antagonist, by way of example and with preference pirfenidone or fresolimumab.

In a preferred embodiment of the invention, the compounds according to the invention are administered in combination with a TNFalpha antagonist, by way of example and with preference adalimumab.

In a preferred embodiment of the invention, the compounds according to the invention are administered in combination with HIF-PH inhibitors, by way of example and with preference molidustat or roxadustat.

In a preferred embodiment of the invention, the compounds according to the invention are used in combination with a serotonin receptor antagonist, by way of example and with preference PRX-08066.

Particular preference is given to combinations of the compounds according to the invention with one or more further active compounds selected from the group consisting of platelet aggregation inhibitors, anticoagulants, profibrinolytic substances, substances which affect the energy metabolism of the heart and mitochondrial function/ROS production, hypotensive drugs, mineralocorticoid receptor antagonists, HMG CoA reductase inhibitors, drugs which modulate lipid metabolism, active compounds which modulate glucose metabolism and active compounds for anxiety and pain therapy such as benzodiazepines and opiates.

The present invention further provides medicaments and pharmaceutical compositions which comprise at least one compound of the invention, typically together with one or more inert, non-toxic, pharmaceutically suitable excipients, and for the use thereof for the aforementioned purposes.

The compounds according to the invention can act systemically and/or locally. For this purpose, they can be administered in a suitable manner, for example by the oral, parenteral, pulmonal, nasal, sublingual, lingual, buccal, rectal, vaginal, dermal, transdermal, conjunctival or otic route, or as an implant or stent.

The compounds according to the invention can be administered in administration forms suitable for these administration routes.

Parenteral administration can be accomplished with avoidance of a resorption step (for example by an intravenous, intraarterial, intracardiac, intraspinal or intralumbar route) or with inclusion of a resorption (for example by an intramuscular, subcutaneous, intracutaneous, percutaneous, intravitreal or intraperitoneal route). Administration forms suitable for parenteral administration include inter alia preparations for injection and infusion in the form of solutions, suspensions, emulsions, lyophilizates or sterile powders.

Suitable administration forms for oral administration are those which function according to the prior art and deliver the inventive compounds rapidly and/or in modified fashion, and which contain the inventive compounds in crystalline and/or amorphized and/or dissolved form, for example tablets (uncoated or coated tablets, for example having enteric coatings or coatings which are insoluble or dissolve with a delay, which control the release of the compound according to the invention), tablets which disintegrate rapidly in the mouth, or films/wafers, films/lyophilizates, capsules (for example hard or soft gelatin capsules), sugar-coated tablets, granules, pellets, powders, emulsions, suspensions, aerosols or solutions.

Suitable administration forms for the other administration routes are, for example, pharmaceutical forms for inhalation (including powder inhalers, nebulizers), nasal drops, solutions or sprays; tablets for lingual, sublingual or buccal administration, films/wafers or capsules, suppositories, eye drops, eye ointments, eyewashes, ocular inserts, ear drops, sprays, powders, washes or tampons, vaginal capsules, aqueous suspensions (lotions, shaking mixtures), lipophilic suspensions, emulsions, microemulsions, ointments, creams, transdermal therapeutic systems (for example patches), milk, pastes, foams, dusting powders, implants or stents.

The compounds according to the invention can be converted to the administration forms mentioned. This can be accomplished in a manner known per se by mixing with pharmaceutically suitable excipients.

These excipients include
fillers and carriers (for example cellulose, microcrystalline cellulose, for example Avicel®, lactose, mannitol, starch, calcium phosphates, for example Di-Cafos®),
ointment bases (for example vaseline, paraffins, triglycerides, waxes, wool wax, wool wax alcohols, lanolin, hydrophilic ointment, polyethylene glycols),
suppository bases (for example polyethylene glycols, cocoa butter, hard fat),
solvents (e.g. water, ethanol, isopropanol, glycerol, propylene glycol, mid-chain triglycerides, fatty oils, liquid polyethylene glycols, paraffins),
surfactants, emulsifiers, dispersants or wetting agents (for example sodium dodecylsulfate, lecithin, phospholipids, fatty alcohols, for example Lanette®, sorbitan fatty acid esters, for example Span®, polyoxyethylene sorbitan fatty acid esters, for example Tween®, polyoxyethylene fatty acid glycerides, for example Cremophor®, polyoxyethylene fatty acid esters, polyoxyethylene fatty alcohol ethers, glycerol fatty acid esters, poloxamers, for example Pluronic®),
buffer substances, and also acids and bases (for example phosphates, carbonates, citric acid, acetic acid, hydrochloric acid, sodium hydroxide, ammonium carbonate, trometamol, triethanolamine),
isotonizing agents (for example glucose, sodium chloride),
adsorbents (for example finely divided silicas),
viscosity-increasing agents, gel formers, thickeners or binders (for example polyvinylpyrrolidone, methyl cellulose, hydroxypropyl methyl cellulose, hydroxypropyl cellulose, carboxymethyl cellulose-sodium, starch, carbomers, polyacrylic acids, for example Carbopol®, alginates, gelatins),
disintegrants (for example modified starch, carboxymethyl cellulose-sodium, sodium starch glycolate, for example Explotab®, crosslinked polyvinylpyrrolidone, croscarmellose-sodium, for example AcDiSol®),
flow regulators, lubricants, glidants and mould release agents (for example magnesium stearate, stearic acid, talc, finely divided silicas, for example Aerosil®),
coating agents (for example sugar, shellac) and film formers for films or diffusion membranes with fast or modified dissolution (for example polyvinylpyrrolidones, for example Kollidon®, polyvinyl alcohol, hydroxypropyl methyl cellulose, hydroxypropyl cellulose, ethyl cellulose, hydroxypropyl methyl cellulose phthalate, cellulose acetate, cellulose acetate phthtalate, polyacrylates, polymethacrylates, for example Eudragit®),
capsule materials (e.g. gelatins, hydroxypropyl methyl cellulose),
synthetic polymers (for example polylactides, polyglycolides, polyacrylates, polymethacrylates, for example Eudragit®, polyvinylpyrrolidones, for example Kollidon®, polyvinyl alcohols, polyvinyl acetates, polyethylene oxides, polyethylene glycols and the copolymers and block copolymers thereof),
plasticizers (for example polyethylene glycols, propylene glycol, glycerol, triacetin, triacetyl citrate, dibutyl phthalate),
penetration enhancers,
stabilizers (e.g. antioxidants, for example ascorbic acid, ascorbyl palmitate, sodium ascorbate, butylhydroxyanisole, butylhydroxytoluene, propyl gallate),
preservatives (for example parabens, sorbic acid, thiomersal, benzalkonium chloride, chlorhexidine acetate, sodium benzoate),
dyes (e.g. inorganic pigments, for example iron oxides, titanium dioxide),
aromas, sweeteners, flavour and/or odour correctors.

Parenteral administration is preferred. Particular preference is given to iv administration, especially in physiological saline.

The present invention further provides pharmaceutical compositions comprising at least one compound according to the invention, typically together with one or more pharmaceutically suitable excipients, and the use thereof according to the present invention.

In general, it has been found to be advantageous in the case of parenteral administration to administer amounts of about 0.001 to 1 mg/kg, preferably about 0.01 to 0.5 mg/kg body weight to achieve effective results. In the case of oral administration the dosage is about 0.01 to 100 mg/kg, preferably about 0.01 to 20 mg/kg and most preferably 0.1 to 10 mg/kg body weight. In the case of intrapulmonary administration, the amount is generally about 0.1 to 50 mg per inhalation.

It may nevertheless be necessary in some cases to deviate from the stated amounts, and specifically as a function of body weight, route of administration, individual response to the active ingredient, nature of the preparation and time at which or interval over which administration takes place. Thus in some cases it may be sufficient to manage with less than the aforementioned minimum amount, while in other cases the upper limit mentioned must be exceeded. In the case of administration of greater amounts, it may be advisable to divide them into several individual doses over the day.

The working examples which follow illustrate the invention. The invention is not restricted to the examples.

Unless stated otherwise, the percentages in the tests and examples which follow are percentages by weight; parts are parts by weight. Solvent ratios, dilution ratios and concentration data for liquid/liquid solutions are based in each case on volume.

### A. EXAMPLES

### Abbreviations and Acronyms

| | |
|---|---|
| GP | General Procedure |
| abs. | absolute |
| AIBN | azobis(isobutyronitrile) |
| aq. | aqueous, aqueous solution |
| br. | broad (in NMR signal) |
| Ex. | Example |
| Bu | butyl |
| c | concentration |
| ca. | circa, about |
| cat. | catalytic |
| CDI | carbonyldiimidazole |
| CI | chemical ionization (in MS) |
| CH | cyclohexane |
| d | doublet (in NMR) |
| d | day(s) |
| TLC | thin layer chromatography |
| DCM | dichloromethane |
| dd | doublet of doublets (in NMR) |
| de | diastereomeric excess |
| DEA | diethylamine |
| dist. | distilled |
| DIPEA | N,N-diisopropylethylamine |
| DMAP | 4-N,N-dimethylaminopyridine |
| DMF | N,N-dimethylformamide |
| DMSO | dimethyl sulfoxide |
| dt | doublet of triplets (in NMR) |
| EDC*HC1 | 1-ethyl-3-(31-dimethylaminopropyl)carbodiimide |
| | hydrochloride |
| ee | enantiomeric excess |
| EA | ethyl acetate |
| EI | electron impact ionization (in MS) |
| ent | enantiomerically pure, enantiomer |
| eq. | equivalent(s) |
| ESI | electrospray ionization (in MS) |
| Et | ethyl |
| GC | gas chromatography |
| GC/MS | gas chromatography-coupled mass spectrometry |
| h | hour(s) |
| HATU | 0-(7-azabenzotriazol-1-yl)-N,N,N′,N′-tetramethyl- |
| | uronium hexafluorophosphate |
| HPLC | high-pressure, high-performance liquid chromatography |
| conc. | concentrated (in the case of a solution) |
| LC | liquid chromatography |
| LC/MS | liquid chromatography-coupled mass spectrometry |
| lit. | literature (reference) |
| m | multiplet (in NMR) |
| M | molar (in solution) |
| Me | methyl |
| min | minute(s) |
| MTBE | methyl t-butyl ether |
| MS | mass spectrometry |
| N | normal (concentration) |
| NIS | N-iodosuccinimide |
| NMR | nuclear magnetic resonance spectrometry |
| q (or quart) | quartet (in NMR) |
| qd | quartet of doublets (in NMR) |
| quant. | quantitative (in chemical yield) |
| quintt | quintet (in NMR) |
| rac | racemic, racemate |
| RP | reverse phase (in HPLC) |
| RT | room temperature |
| Rt | retention time (in HPLC, LC/MS) |
| s | singlet (in NMR) |
| sept | septet (in NMR) |
| SFC | supercritical liquid chromatography |
| t | triplet (in NMR) |
| tBu | tert-butyl |
| td | triplet of doublets (in NMR) |
| TEA | Triethyl amine |
| TFA | trifluoroacetic acid |
| THF | tetrahydrofuran |
| UV | ultraviolet spectrometry |
| cf. | see |
| v/v | volume to volume ratio (of a solution) |
| xantphos | 9,9-dimethyl-4,5-bis(diphenylphosphino)xanthene |
| tog. | together |

HPLC, GC-MS and LC-MS Methods

Method 1:

Instrument: Waters Single Quad MS System; instrument Waters UPLC Acquity; column: Waters BEH C18 1.7 μm 50×2.1 mm; mobile phase A: 1 l of water+1.0 ml of (25% strength ammonia)/1, mobile phase B: 1 l of acetonitrile; gradient: 0.0 min 92% A→0.1 min 92% A→1.8 min 5% A→3.5 min 5% A; oven: 50° C.; flow rate: 0.45 ml/min; UV detection: 210 nm (208-400 nm).

Method 2:

MS instrument type: Thermo Scientific FT-MS; instrument type UHPLC+: Thermo Scientific UltiMate 3000; column: Waters, HSST3, 2.1×75 mm, C18 1.8 μm; mobile phase A: 11 of water+0.01% formic acid; mobile phase B: 1 l of acetonitrile+0.01% formic acid; gradient: 0.0 min 10% B→2.5 min 95% B→3.5 min 95% B; oven: 50° C.; flow rate: 0.90 ml/min; UV detection: 210 nm/optimum integration path 210-300 nm.

Method 3:

Instrument: Waters ACQUITY SQD UPLC System; column: Waters Acquity UPLC HSS T3 1.8 μm 50×1 mm; mobile phase A: 1 l of water+0.25 ml of 99% strength formic acid, mobile phase B: 1 l of acetonitrile+0.25 ml of 99% strength formic acid; gradient: 0.0 min 90% A→1.2 min 5% A→2.0 min 5% A; oven: 50° C.; flow rate: 0.40 ml/min; UV detection: 208-400 nm.

Method 4:

Instrument: Agilent MS Quad 6150; HPLC: Agilent 1290; column: Waters Acquity UPLC HSS T3 1.8 μm 50×2.1 mm; mobile phase A: 1 l of water+0.25 ml of 99% strength formic acid, mobile phase B: 1 l of acetonitrile+0.25 ml of 99% strength formic acid; gradient: 0.0 min 90% A→0.3 min 90% A→1.7 min 5% A→3.0 min 5% A; oven: 50° C.; flow rate: 1.20 ml/min; UV detection: 205-305 nm.

Method 5:

MS instrument type: ThermoFisherScientific LTQ-Orbitrap-XL; HPLC instrument type: Agilent 1200SL; column: Agilent, POROSHELL 120, 3×150 mm, SB—C18 2.7 μm; mobile phase A: 1 l of water+0.1% trifluoroacetic acid; mobile phase B: 1 l of acetonitrile+0.1% trifluoroacetic acid; gradient: 0.0 min 2% B→0.3 min 2% B→5.0 min 95% B→10.0 min 95% B; oven: 40° C.; flow rate: 0.75 ml/min; UV detection: 210 nm.

Method 6:

Instrument: Waters ACQUITY SQD UPLC System; column: Waters Acquity UPLC HSS T3 1.8 μm 50×1 mm; mobile phase A: 1 l of water+0.25 ml of 99% strength formic acid, mobile phase B: 1 l of acetonitrile+0.25 ml of 99% strength formic acid; gradient: 0.0 min 95% A→6.0 min 5% A→7.5 min 5% A; oven: 50° C.; flow rate: 0.35 ml/min; UV detection: 210-400 nm.

Further Details:

In the case of purifications of compounds of the invention by chromatography, particularly by column chromatography, prepacked silica gel cartridges, for example Biotage SNAP cartridges, KP-Sil® or KP-NH, are used in combination with a Biotage system (SP4 or Isolera Four). Eluents employed are gradients of hexane/ethyl acetate or dichloromethane/methanol.

In the case of purifications of compounds of the invention by preparative HPLC by the above-described methods in which the eluents contain additives, for example trifluoroacetic acid or formic acid, the compounds of the invention can be obtained in salt form, for example as trifluoroacetate or formate salt, if the compounds of the invention contain a sufficiently basic functionality. Such a salt can be converted to the corresponding free base by various methods known to the person skilled in the art.

In the case of the synthesis intermediates and working examples of the invention described hereinafter, any compound specified in the form of a salt of the corresponding base or acid is generally a salt of unknown exact stoichiometric composition, as obtained by the respective preparation and/or purification process. Unless specified in more detail, additions to names and structural formulae, such as “hydrochloride”, “trifluoroacetate”, “sodium salt” or “x hydrochloric acid, “x CF₃COOH”, “x Na” should not therefore be understood in a stoichiometric sense in the case of such salts, but have merely descriptive character with regard to the salt-forming components present therein.

This applies correspondingly if synthesis intermediates or working examples or salts thereof were obtained in the form of solvates, for example hydrates, of unknown stoichiometric composition (if they are of a defined type) by the preparation and/or purification processes described.

Furthermore, the secondary amides according to the invention may be present as rotational isomers/isomer mixtures, in particular in NMR studies. Purity figures are generally based on corresponding peak integrations in the LC/MS chromatogram, but may additionally also have been determined with the aid of the ¹H NMR spectrum. If no purity is indicated, the purity is generally 100% according to automated peak integration in the LC/MS chromatogram, or the purity has not been determined explicitly.

Stated yields in % of theory are generally corrected for purity if a purity of <100% is indicated. In solvent-containing or contaminated batches, the formal yield may be “>100%”; in these cases the yield is not corrected for solvent or purity.

In all ¹H NMR spectra data, the chemical shifts δ [ppm]=are stated in ppm.

The multiplicities of proton signals in ¹H NMR spectra reported in the paragraphs which follow represent the signal form observed in each case and do not take account of any higher-order signal phenomena. In general, the stated chemical shift refers to the centre of the signal in question. In the case of broad multiplets, an interval is given. Signals obscured by solvent or water were either tentatively assigned or have not been listed. Significantly broadened signals—caused, for example, by rapid rotation of molecular moieties or because of exchanging protons—were likewise assigned tentatively (often referred to as a broad multiplet or broad singlet) or are not listed.

Melting points and melting ranges, if stated, are uncorrected.

The ¹H NMR data of selected synthesis intermediates and working examples are stated in the form of ¹H NMR peak lists. For each signal peak, first the δ [ppm]=value in ppm and then the signal intensity in round brackets are listed. The δ [ppm]=value/signal intensity number pairs for different signal peaks are listed with separation from one another by commas. The peak list for an example therefore takes the following form: δ [ppm]=₁ (intensity₁), δ [ppm]=₂ (intensity₂), . . . , δ [ppm]=ᵢ (intensityᵢ), . . . , δ [ppm]=ₙ(intensityₙ).

The intensity of sharp signals correlates with the height of the signals in a printed example of an NMR spectrum in cm and shows the true ratios of the signal intensities in comparison with other signals. In the case of broad signals, several peaks or the middle of the signal and the relative intensity thereof may be shown in comparison to the most intense signal in the spectrum. The lists of the ¹H NMR peaks are similar to the conventional ¹H NMR printouts and thus usually contain all peaks listed in a conventional NMR interpretation. In addition, like conventional ¹H NMR printouts, they may show solvent signals, signals of stereoisomers of the target compounds which are likewise provided by the invention, and/or peaks of impurities. The peaks of stereoisomers of the target compounds and/or peaks of impurities usually have a lower intensity on average than the peaks of the target compounds (for example with a purity of >90%).

Such stereoisomers and/or impurities may be typical of the particular preparation process. Their peaks can thus help in identifying reproduction of our preparation process with reference to “by-product fingerprints”. An expert calculating the peaks of the target compounds by known methods (MestreC, ACD simulation, or using empirically evaluated expected values) can, if required, isolate the peaks of the target compounds, optionally using additional intensity filters. This isolation would be similar to the peak picking in question in conventional ¹H NMR interpretation. A detailed description of the presentation of NMR data in the form of peak lists can be found in the publication “Citation of NMR Peaklist Data within patent applications” (cf. Research Disclosure Database Number 605005, 2014, 1 Aug. 2014 or http://www.researchdisclosure.com/searching-disclosures). In the peak picking routine described in Research Disclosure Database Number 605005, the parameter “MinimumHeight” can be set between 1% and 4%. Depending on the type of chemical structure and/or depending on the concentration of the compound to be analysed, it may be advisable to set the parameters “MinimumHeight” to values of <1%.

All reactants or reagents whose preparation is not described explicitly hereinafter were purchased commercially from generally accessible sources. For all other reactants or reagents whose preparation likewise is not described hereinafter and which were not commercially obtainable or were obtained from sources which are not generally accessible, a reference is given to the published literature in which their preparation is described.

### STARTING COMPOUNDS AND INTERMEDIATES

### Example 1A

### Methyl imidazo[1,2-a]pyridine-7-carboxylate

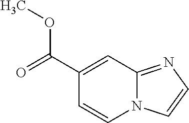

2-Bromo-1,1-dimethoxyethane (140 ml, 1.2 mol) was initially charged in 365 ml of water and concentrated hydrochloric acid (8.5 ml) and stirred at 85° C. for one hour. The mixture was cooled and solid sodium bicarbonate (104 g, 1.23 mol) was added carefully (pH=8). Methyl 2-aminoisonicotinate (125 g, 822 mmol) was added and the suspension was stirred at 100° C. for three hours. The solution then present was cooled to room temperature and stirred overnight. The re-formed suspension was filtered with suction and the residue was washed repeatedly with water. The solid (title compound) was dried in a vacuum drying cabinet at 40° C. overnight. The filtrate was adjusted to pH 10 using aqueous sodium hydroxide solution and saturated with sodium chloride. The mixture was extracted three times with in each case 500 ml of ethyl acetate. The combined organic phases were dried over magnesium sulphate, filtered and concentrated. The residue obtained in this manner (title compound) was dried under high vacuum. The two charges of title compound were combined. This gave a total of 108 g (75% of theory, 100% purity) of the title compound.

LC-MS (Method 1): Rₜ=0.95 min; MS (ESIpos): m/z=177 [M+H]⁺

¹H-NMR (400 MHz, DMSO-d6) δ [ppm]: 3.325 (16.00), 7.317 (3.44), 7.320 (3.29), 7.334 (3.63), 7.337 (3.52), 7.799 (8.51), 8.156 (15.65), 8.650 (5.65), 8.667 (5.53).

### Example 2A

### Methyl 3-iodoimidazo[1,2-a]pyridine-7-carboxylate

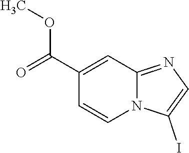

Methyl imidazo[1,2-a]pyridine-7-carboxylate (51.1 g, 290 mmol) was dissolved in 2.51 of acetonitrile. 1-Iodopyrrolidine-2,5-dione (68.5 g, 304 mmol) was added and the mixture was stirred at room temperature for four days. The mixture was added to 3.5 l of water, adjusted to pH 8 using solid sodium bicarbonate and stirred for 15 minutes. The precipitate was filtered off with suction and washed once with saturated aqueous sodium bicarbonate solution and once with water. The solid was then suspended in acetonitrile and sucked dry. The solid was dried under reduced pressure for two days. This gave a total of 81 g (93% of theory) of the title compound.

LC-MS (Method 2): Rₜ=1.30 min; MS (ESIpos): m/z=303 [M+H]⁺

¹H-NMR (400 MHz, DMSO-d6) δ [ppm]: 3.896 (0.56), 3.909 (16.00), 7.453 (1.52), 7.457 (1.59), 7.471 (1.63), 7.475 (1.70), 7.944 (3.59), 8.162 (1.97), 8.436 (2.14), 8.454 (2.16).

### Example 3A

### 3-(3,5-Dimethyl-1,2-oxazol-4-yl)imidazo[1,2-a]pyridine-7-carboxylic acid

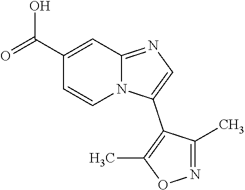 [SMILES: Cc1noc(C)c1-c1cnc2cc(C(=O)O)ccn12]

Preparative Method 1:

Methyl 3-iodoimidazo[1,2-a]pyridine-7-carboxylate (2.80 g, 9.27 mmol) and tetrakis(triphenylphosphine)palladium(0) (536 mg, 463 μmol) were initially charged in 75 ml of 1,2-dimethoxyethane, and (3,5-dimethyl-1,2-oxazol-4-yl)boronic acid (3.27 g, 23.2 mmol), potassium carbonate (2.56 g, 18.5 mmol) and 37 ml of water were added. The mixture was stirred at 75° C. for 4.5 hours. More (3,5-dimethyl-1,2-oxazol-4-yl)boronic acid (653 mg, 4.64 mmol) and tetrakis(triphenylphosphine)palladium(0) (268 mg, 232 μmol) were added and the mixture was stirred at 75° C. for 24 hours. The reaction mixture was purified by silica gel chromatography (340 g Snap Cartridge Biotage®; Biotage-Isolera-One; dichloromethane/methanol 1:1+0.45% acetic acid). The product fractions were combined and concentrated. This gave a total of 1230 mg (52% of theory, 100% purity) of the title compound.

LC-MS (Method 2): Rₜ=0.56 min; MS (ESIpos): m/z=258 [M+H]⁺

¹H-NMR (400 MHz, DMSO-d6) δ [ppm]: −0.008 (1.11), 0.008 (1.11), 1.563 (0.48), 1.574 (0.43), 2.128 (15.93), 2.336 (16.00), 3.243 (0.57), 3.896 (0.47), 7.337 (1.56), 7.342 (1.62), 7.355 (1.62), 7.359 (1.70), 7.920 (4.55), 8.195 (2.21), 8.235 (2.02), 8.253 (1.97).

Preparative Method 2:

Methyl 3-(3,5-dimethyl-1,2-oxazol-4-yl)imidazo[1,2-a]pyridine-7-carboxylate (3.85 g, 14.2 mmol) was initially charged in 90 ml of tetrahydrofuran/methanol, aqueous sodium hydroxide solution (28 ml, 1.0 M, 28 mmol) was added and the mixture was stirred at room temperature for 30 minutes. The organic solvents were removed on a rotary evaporator and the residue was acidified with 4 N hydrochloric acid. The precipitated solid was filtered off with suction and dried under high vacuum. This gave 2.42 g (100% pure, 66% of theory) of the title compound.

LC-MS (Method 2): Rₜ=0.54 min; MS (ESIpos): m/z=258 [M+H]⁺

¹H-NMR (400 MHz, DMSO-d6) δ [ppm]: −0.008 (1.21), 0.008 (1.15), 2.128 (15.97), 2.336 (16.00), 7.340 (1.54), 7.344 (1.56), 7.358 (1.57), 7.362 (1.61), 7.927 (4.93), 8.199 (2.56), 8.241 (2.00), 8.260 (1.89), 13.365 (0.82).

The filtrate was concentrated and the residue was stirred with methanol. The insoluble salts were filtered off and discarded. The filtrate was re-concentrated and the residue was stirred with acetonitrile. The solids were filtered off with suction and dried under high vacuum. This gave 1.35 g (100% pure, 37% of theory) of the title compound.

LC-MS (Method 2): Rₜ=0.57 min; MS (ESIpos): m/z=258 [M+H]⁺

### Example 4A

### Sodium 3-(3,5-dimethyl-1,2-oxazol-4-yl)imidazo[1,2-a]pyridine-7-carboxylate

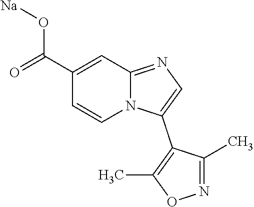

Methyl 3-(3,5-dimethyl-1,2-oxazol-4-yl)imidazo[1,2-a]pyridine-7-carboxylate (680 mg, 2.51 mmol) was initially charged in 16 ml of tetrahydrofuran/methanol (3:1), aqueous sodium hydroxide solution (5.0 ml, 1.0 M, 5.0 mmol) was added and the mixture was stirred at room temperature overnight. The reaction mixture was neutralized with 1 N hydrochloric acid and concentrated. The residue was stirred with methanol and the insoluble salts were discarded. The filtrate was concentrated and the residue was stirred with acetonitrile. The solids were filtered off with suction and dried under high vacuum. This gave a total of 630 mg (90% of theory, 100% purity) of the title compound.

LC-MS (Method 2): Rₜ=0.56 min; MS (ESIpos): m/z=258 [M+2H-Na]⁺

### Example 5A

### Methyl 3-(3,5-dimethyl-1,2-oxazol-4-yl)imidazo[1,2-a]pyridine-7-carboxylate

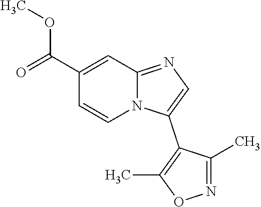 [SMILES: COC(=O)c1ccn2c(-c3c(C)noc3C)cnc2c1]

Methyl 3-iodoimidazo[1,2-a]pyridine-7-carboxylate (50.0 g, 166 mmol) was initially charged in 2.5 l of N,N-dimethylformamide. (3,5-Dimethyl-1,2-oxazol-4-yl)boric acid (46.7 g, 331 mmol) and caesium fluoride (75.4 g, 497 mmol) were added. For 10 minutes, argon was passed through the reaction mixture. [1,1-Bis(diphenylphosphino)ferrocene]dichloropalladium(II) (13.5 g, 16.6 mmol) was added. The mixture was heated to 90° C. and stirred for three hours. The mixture was added to water and a little saturated aqueous sodium bicarbonate solution and extracted three times with ethyl acetate. The combined organic phases were washed with water and saturated aqueous sodium chloride solution, dried over magnesium sulfate, filtered and concentrated. The residue was purified by silica gel chromatography (1500 g column; Biotage-Isolera-One; gradient ethyl acetate in cyclohexane 20-100%). The product fractions were combined and concentrated. This gave a total of 32.2 g (72% of theory, 100% purity) of the title compound.

LC-MS (Method 2): Rₜ=1.13 min; MS (ESIpos): m/z=272 [M+H]⁺

### Example 6A

### tert-Butyl (imidazo[1,2-a]pyridin-2-ylmethyl)carbamate

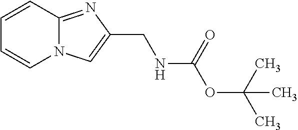 [SMILES: CC(C)(C)OC(=O)NCc1cn2ccccc2n1]

1-(Imidazo[1,2-a]pyridin-2-yl)methanamine dihydrochloride hydrate (1.00 g, 4.20 mmol) was initially charged in 15 ml of tetrahydrofuran and cooled to 0° C. At this temperature, triethylamine (1.8 ml, 13 mmol), 4-dimethylaminopyridine (77.0 mg, 630 μmol) and di-tert-butyl dicarbonate (1.0 ml, 4.4 mmol) were added in succession. The reaction mixture was stirred at room temperature overnight. Water was added and the mixture was extracted with ethyl acetate. The organic phase was washed with water and saturated aqueous sodium chloride solution, dried over magnesium sulfate, filtered and concentrated. This gave 422 mg (41% of theory, 69% purity) of the title compound.

LC-MS (Method 1): Rₜ=1.21 min; MS (ESIpos): m/z=248 [M+H]⁺

### Example 7A

### 2-{[(tert-Butoxycarbonyl)amino]methyl}-1-methylimidazo[1,2-a]pyridin-1-ium iodide

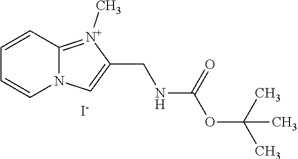 [SMILES: C[n+]1c(CNC(=O)OC(C)(C)C)cn2ccccc21 ; [I-]]

tert-Butyl (imidazo[1,2-a]pyridin-2-ylmethyl)carbamate (963 mg, 3.89 mmol) was initially charged in 18 ml of tetrahydrofuran, iodomethane (1.1 ml, 18 mmol) was added and the mixture was stirred at room temperature overnight. The precipitated solid was filtered off with suction, washed with tetrahydrofuran and dried under high vacuum. This gave 1.36 g (87% of theory, 97% purity) of the title compound.

LC-MS (Method 3): Rₜ=0.43 min; MS (ESIpos): m/z=262 [M-I]⁺

### Example 8A

### 2-(Aminomethyl)-1-methylimidazo[1,2-a]pyridin-1-ium iodide hydrochloride (1:1:1)

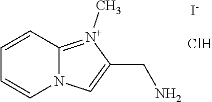

2-{[(tert-Butoxycarbonyl)amino]methyl}-1-methylimidazo[1,2-a]pyridin-1-ium iodide (1.36 g, 3.49 mmol) was initially charged in 35 ml of dichloromethane, 4 N hydrochloric acid in dioxane (8.7 ml, 4.0 M, 35 mmol) was added and the mixture was stirred at room temperature for 4 hours. The solid was filtered off with suction and washed with dichloromethane. The solid was dried under high vacuum. This gave 830 mg (73% of theory, 100% purity) of the title compound.

LC-MS (Method 3): Rₜ=0.14 min; MS (ESIneg): m/z=162 [M-I-HCl]⁺

### Example 9A

### 1-[2-(1,3-Dioxo-1,3-dihydro-2H-isoindol-2-yl)ethyl]-4-(methylamino)pyridinium bromide

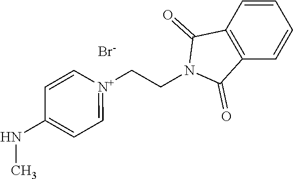 [SMILES: CNc1cc[n+](CCN2C(=O)c3ccccc3C2=O)cc1 ; [Br-]]

N-Methylpyridine-4-amine (5.00 g, 46.2 mmol) was initially charged in 100 ml of N,N-dimethylformamide, 2-(2-bromoethyl)-1H-isoindole-1,3(2H)-dione (11.7 g, 46.2 mmol) was added and the mixture was stirred at 110° C. overnight. The precipitated solid was filtered off with suction, washed with methyl tert-butyl ether and dried under high vacuum. This gave 13.7 g (82% of theory, 100% purity) of the title compound.

LC-MS (Method 3): Rₜ=0.39 min; MS (ESIpos): m/z=282 [M-Br]⁺

### Example 10A

### Methyl 3-(1,4-dimethyl-1H-pyrazol-5-yl)imidazo[1,2-a]pyridine-7-carboxylate

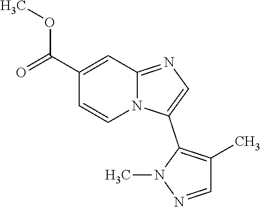 [SMILES: COC(=O)c1ccn2c(-c3c(C)cnn3C)cnc2c1]

Methyl 3-iodoimidazo[1,2-a]pyridine-7-carboxylate (250 mg, 828 μmol), 1,4-dimethyl-5-(4,4,5,5-tetramethyl-1,3,2-dioxaborolan-2-yl)-1H-pyrazole (221 mg, 993 μmol) and potassium carbonate (377 mg, 2.73 mmol) were initially charged in 5 ml of 1,4-dioxane, and the mixture was degassed with argon for 10 minutes. [1,1-Bis(diphenylphosphino)ferrocene]dichloropalladium/dichloromethane complex (33.8 mg, 41.4 μmol) was then added and the mixture was stirred at 110° C. overnight. The reaction mixture was concentrated and the residue was taken up in ethyl acetate and washed with water and saturated aqueous sodium chloride solution. The organic phase was dried over sodium sulfate and concentrated. The residue was applied to Isolute and purified by column chromatography (50 g Biotage Snap Cartridge Ultra®; Biotage-Isolera-One®; dichloromethane/methanol gradient 2% methanol-20% methanol; flow rate: 100 ml/min). The product fractions were combined and concentrated. This gave 122 mg (40% of theory, 74% purity) of the title compound.

LC-MS (Method 3): Rₜ=0.61 min; MS (ESIpos): m/z=271 [M+H]⁺

### Example 11A

### 1-(2-Ammonioethyl)-4-(methylamino)pyridiniumdibromide

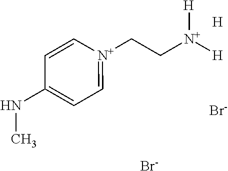

1-[2-(1,3-Dioxo-1,3-dihydro-2H-isoindol-2-yl)ethyl]-4-(methylamino)pyridiniumbromide (13.7 g, 37.8 mmol) in 50 ml of 48% strength aqueous hydrogen bromide solution was heated under reflux at 100° C. for two days. The reaction mixture was cooled and the solid formed was filtered off and discarded. The filtrate was concentrated and the residue was stirred with tetrahydrofuran. The solid was filtered off, washed with tetrahydrofuran and dried under high vacuum. This gave 11.5 g (97% of theory) of the title compound.

LC-MS (Method 1): Rₜ=0.22 min; MS (ESIpos): m/z=152 [M−HBr-Br]⁺

¹H-NMR (400 MHz, DMSO-d6) δ [ppm]: −0.008 (2.31), 0.008 (2.34), 2.524 (1.28), 2.911 (15.94), 2.923 (16.00), 4.348 (2.67), 4.363 (4.93), 4.378 (2.67), 6.893 (1.82), 6.900 (2.48).

### Example 12A

### 3-(1,4-Dimethyl-1H-pyrazol-5-yl)imidazo[1,2-a]pyridine-7-carboxylic acid

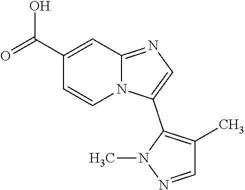 [SMILES: Cc1cnn(C)c1-c1cnc2cc(C(=O)O)ccn12]

Methyl 3-(1,4-dimethyl-1H-pyrazol-5-yl)imidazo[1,2-a]pyridine-7-carboxylate (122 mg, 451 μmol) was initially charged in 7 ml of tetrahydrofuran/water (3:1), lithium hydroxide (21.6 mg, 903 μmol) was added and the mixture was stirred at room temperature for two hours. The reaction mixture was concentrated and the residue was purified directly by preparative HPLC (column: Chromatorex C18 10 μm, 250×30 mm, mobile phase A=water, B=acetonitrile; gradient: 0.0 min 5% B; 3 min 5% B; 20 min 50% B; 23 min 100% B; 26 min 5% B; flow rate: 50 ml/min; 0.1% formic acid). The product-containing fractions were combined, concentrated and dried under high vacuum. This gave 59 mg (51% of theory, 100% purity) of the title compound.

LC-MS (Method 2): Rₜ=0.62 min; MS (ESIpos): m/z=257 [M+H]⁺

### Example 13A

### 1-[3-(1,3-Dioxo-1,3-dihydro-2H-isoindol-2-yl)propyl]-4-(methylamino)pyridinium bromide

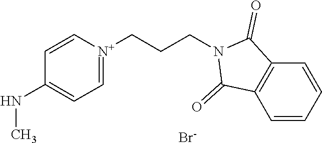 [SMILES: CNc1cc[n+](CCCN2C(=O)c3ccccc3C2=O)cc1 ; [Br-]]

N-Methylpyridine-4-amine (2.00 g, 18.5 mmol) was initially charged in 20 ml of N,N-dimethylformamide, 2-(3-bromopropyl)-1H-isoindole-1,3(2H)-dione (4.96 g, 18.5 mmol) was added and the mixture was stirred at 110° C. overnight. The precipitated solid was filtered off with suction and washed with methyl tert-butyl ether. The solid was dried under high vacuum. This gave 5.62 g (81% of theory, 100% purity) of the title compound.

LC-MS (Method 2): Rₜ=0.83 min; MS (ESIpos): m/z=296 [M-Br]⁺

### Example 14A

### 1-(3-Aminopropyl)-4-(methylamino)pyridinium bromide hydrobromide (1:1:1)

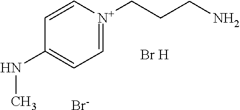

1-[3-(1,3-Dioxo-1,3-dihydro-2H-isoindol-2-yl)propyl]-4-(methylamino)pyridinium bromide (5.62 g, 14.9 mmol) in 21 ml of 48% strength aqueous hydrogen bromide solution was heated under reflux at 100° C. overnight. The reaction mixture was cooled and the solid formed was filtered off and discarded. The filtrate was concentrated and the residue was stirred with tetrahydrofuran. The solid was filtered off, washed with tetrahydrofuran and dried under high vacuum. This gave 3.75 g (77% of theory) of the title compound.

LC-MS (Method 1): Rₜ=1.41 min; MS (ESIpos): m/z=166 [M−HBr-Br]⁺

¹H-NMR (400 MHz, DMSO-d6) δ [ppm]: −0.008 (1.01), 2.017 (0.82), 2.035 (2.75), 2.053 (3.90), 2.072 (2.94), 2.090 (0.99), 2.755 (0.47), 2.770 (1.74), 2.786 (2.82), 2.805 (2.84), 2.821 (1.64), 2.835 (0.46), 2.882 (0.57), 2.898 (16.00), 2.910 (15.96), 4.222 (3.36), 4.239 (6.63), 4.256 (3.21), 6.904 (1.78), 6.911 (3.39), 6.924 (6.38), 6.939 (3.56), 6.945 (1.86), 7.839 (2.89), 8.153 (2.85), 8.171 (2.83), 8.356 (2.75), 8.373 (2.78), 8.725 (1.80), 8.736 (1.82).

### Example 15A

### Methyl 3-(1-ethyl-1H-pyrazol-5-yl)imidazo[1,2-a]pyridine-7-carboxylate

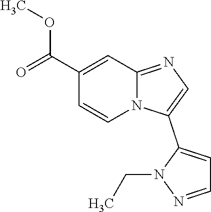 [SMILES: CCn1nccc1-c1cnc2cc(C(=O)OC)ccn12]

Methyl 3-iodoimidazo[1,2-a]pyridine-7-carboxylate (250 mg, 828 μmol), 1-ethyl-5-(4,4,5,5-tetramethyl-1,3,2-dioxaborolan-2-yl)-1H-pyrazole (221 mg, 993 μmol) and potassium carbonate (377 mg, 2.73 mmol) were initially charged in 5 ml of 1,4-dioxane, and the mixture was degassed with argon for 10 minutes. [1,1-Bis(diphenylphosphino)ferrocene]dichloropalladium/dichloromethane complex (33.8 mg, 41.4 μmol) was then added and the mixture was stirred at 110° C. overnight. The reaction mixture was concentrated. The residue was taken up in ethyl acetate and washed with water and saturated aqueous sodium chloride solution. The organic phase was dried over sodium sulfate, filtered and concentrated. The residue was applied to Isolute and purified by column chromatography (25 g Biotage Snap Cartridge Ultra®; Biotage-Isolera-One®; dichloromethane/methanol gradient 2% methanol-10% methanol). The product fractions were combined and concentrated. This gave 143 mg (47% of theory, 73% purity) of the title compound.

LC-MS (Method 2): Rₜ=1.18 min; MS (ESIpos): m/z=271 [M+H]⁺

### Example 16A

### 3-(1-Ethyl-1H-pyrazol-5-yl)imidazo[1,2-a]pyridine-7-carboxylic acid

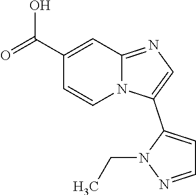 [SMILES: CCn1nccc1-c1cnc2cc(C(=O)O)ccn12]

Methyl 3-(1-ethyl-1H-pyrazol-5-yl)imidazo[1,2-a]pyridine-7-carboxylate (143 mg, 529 μmol) was initially charged in 8 ml of tetrahydrofuran/water (3:1), lithium hydroxide (25.3 mg, 1.06 mmol) was added and the mixture was stirred at room temperature overnight. The reaction mixture was diluted with water and acidified with 1N hydrochloric acid. The mixture was washed with ethyl acetate. The aqueous phase was removed, concentrated and purified by preparative HPLC (column: Chromatorex C18 10 μm, 250×30 mm, mobile phase A=water, B=acetonitrile; gradient: 0.0 min 5% B; 3 min 5% B; 20 min 50% B; 23 min 100% B; 26 min 5% B; flow rate: 50 ml/min; 0.1% formic acid). The product-containing fractions were combined, concentrated and dried under high vacuum. This gave 39 mg (26% of theory, 89% purity) of the title compound.

LC-MS (Method 3): Rₜ=0.33 min; MS (ESIpos): m/z=257 [M+H]⁺

### Example 17A

### Methyl 3-(2-methoxypyridin-3-yl)imidazo[1,2-a]pyridine-7-carboxylate

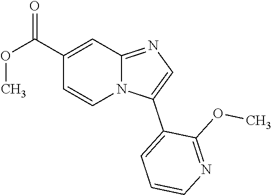 [SMILES: COC(=O)c1ccn2c(-c3cccnc3OC)cnc2c1]

Under argon, methyl 3-bromoimidazo[1,2-a]pyridine-7-carboxylate (150 mg, 588 μmol), (2-methoxypyridin-3-yl)boronic acid (135 mg, 882 μmol) and tetrakis(triphenylphosphine)palladium(0) (6.80 mg, 5.88 μmol) were initially charged in 6.7 ml of N,N-dimethylformamide. 2 M aqueous sodium carbonate solution (1.5 ml, 2.0 M, 2.9 mmol) was then added and the mixture was shaken at 130° C. for 75 minutes. The reaction mixture was acidified with formic acid and filtered through a syringe filter and the filtrate was purified by preparative HPLC (column: Chromatorex C18 10 μm, 125×40 mm, mobile phase A=water, B=acetonitrile; gradient: 0.0 min 5% B; 3 min 5% B; 20 min 50% B; 23 min 100% B; 26 min 5% B; flow rate: 50 ml/min; 0.1% formic acid). The product-containing fractions were combined, concentrated and dried under high vacuum. This gave 30.5 mg (18% of theory, 100% purity) of the title compound.

LC-MS (Method 2): Rₜ=1.23 min; MS (ESIpos): m/z=284 [M+H]⁺

### Example 18A

### 3-(2-Methoxypyridin-3-yl)imidazo[1,2-a]pyridine-7-carboxylic acid

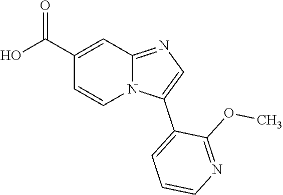 [SMILES: COc1ncccc1-c1cnc2cc(C(=O)O)ccn12]

Methyl 3-(2-methoxypyridine-3-yl)imidazo[1,2-a]pyridine-7-carboxylate (30.5 mg, 108 μmol) was initially charged in 2.2 ml of tetrahydrofuran/water (3:1) and 1M aqueous lithium hydroxide solution (1.1 ml, 1.0 M, 1.1 mmol) was added. The reaction mixture was stirred at room temperature for 1.5 hours. The mixture was concentrated. A little water was added to the residue and the mixture was acidified to pH 3-4 and then purified by preparative HPLC (column: Chromatorex C18 10 μm, 125×40 mm, mobile phase A=water, B=acetonitrile; gradient: 0.0 min 5% B; 3 min 5% B; 20 min 50% B; 23 min 100% B; 26 min 5% B; flow rate: 50 ml/min; 0.1% formic acid). The product-containing fractions were combined, concentrated and dried under high vacuum. This gave 25.2 mg (87% of theory, 100% purity) of the title compound.

LC-MS (Method 4): Rₜ=0.53 min; MS (ESIpos): m/z=270 [M+H]⁺

### Example 19A

### 1-[2-(1,3-Dioxo-1,3-dihydro-2H-isoindol-2-yl)ethyl]-3-(methylamino)pyridinium bromide

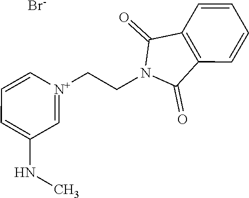 [SMILES: CNc1ccc[n+](CCN2C(=O)c3ccccc3C2=O)c1 ; [Br-]]

N-Methylpyridine-3-amine (1.00 g, 9.25 mmol) was initially charged in 20 ml of N,N-dimethylformamide, 2-(2-bromoethyl)-1H-isoindole-1,3(2H)-dione (2.35 g, 9.25 mmol) was added and the mixture was stirred at 110° C. overnight. The mixture was concentrated and stirred with dichloromethane. The solid was filtered off with suction, washed with dichloromethane and dried under high vacuum. This gave 1.8 g (54% of theory, 100% purity) of the title compound.

LC-MS (Method 2): Rₜ=0.68 min; MS (ESIpos): m/z=282 [M-Br]⁺

¹H-NMR (600 MHz, DMSO-d6) δ [ppm]: 2.682 (7.73), 2.690 (7.71), 4.135 (2.25), 4.143 (3.24), 4.152 (2.36), 4.689 (2.34), 4.697 (3.14), 4.706 (2.20), 7.185 (1.37), 7.194 (1.35), 7.613 (1.01), 7.628 (1.96), 7.652 (1.73), 7.661 (1.77), 7.666 (1.00), 7.676 (0.93), 7.840 (0.47), 7.854 (16.00), 7.869 (0.40), 8.208 (2.03), 8.217 (1.98), 8.244 (2.94).

### Example 20A

### 1-(2-Ammonioethyl)-3-(methylamino)pyridinium dibromide

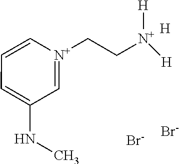

1-[2-(1,3-Dioxo-1,3-dihydro-2H-isoindol-2-yl)ethyl]-3-(methylamino)pyridinium bromide (1.80 g, 4.97 mmol) in 6.6 ml of 48% strength aqueous hydrogen bromide solution was heated under reflux at 100° C. overnight. The reaction mixture was cooled and the solid formed was filtered off and discarded. The filtrate was concentrated and the residue was stirred with tetrahydrofuran. The solid was filtered off, washed with tetrahydrofuran and dried under high vacuum. This gave 1.7 g (82% of theory, 75% purity) of the title compound.

¹H-NMR (500 MHz, DCOOD) 6 [ppm]: 2.906 (1.56), 3.896 (0.56), 4.981 (0.51), 8.116 (3.20), 10.224 (16.00).

### Example 21A

### 4-Amino-1-[2-(1,3-dioxo-1,3-dihydro-2H-isoindol-2-yl)ethyl]-2-methylpyridinium bromide

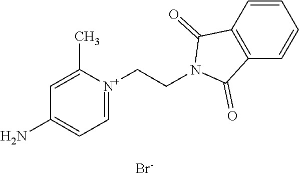 [SMILES: Cc1cc(N)cc[n+]1CCN1C(=O)c2ccccc2C1=O ; [Br-]]

2-Methylpyridine-4-amine (1.00 g, 9.25 mmol) was initially charged in 20 ml of N,N-dimethylformamide, 2-(2-bromoethyl)-1H-isoindole-1,3(2H)-dione (2.35 g, 9.25 mmol) was added and the mixture was stirred at 110° C. overnight. The reaction mixture was substantially concentrated and stirred with acetonitrile. The solid was filtered off with suction and washed with acetonitrile at −10° C. This gave 970 mg (28% of theory, 98% purity) of the title compound.

LC-MS (Method 2): Rₜ=0.66 min; MS (ESIpos): m/z=282 [M-Br]⁺

¹H-NMR (400 MHz, DMSO-d6) δ [ppm]: −0.008 (0.78), 2.475 (5.44), 2.648 (16.00), 4.036 (1.87), 4.051 (3.75), 4.066 (2.06), 4.638 (1.98), 4.653 (3.56), 4.668 (1.81), 6.239 (3.70), 7.483 (0.43), 7.505 (0.93), 7.522 (1.12), 7.528 (1.17), 7.544 (2.36), 7.550 (2.05), 7.606 (3.44), 7.628 (2.01), 7.853 (2.07), 7.864 (2.15), 7.871 (2.47), 7.876 (8.50), 7.884 (8.57), 7.889 (2.67), 7.895 (2.05), 7.900 (0.92), 7.906 (1.10), 7.946 (3.23), 7.952 (3.23).

### Example 22A

### 4-Amino-1-(2-ammonioethyl)-2-methylpyridinium dibromide

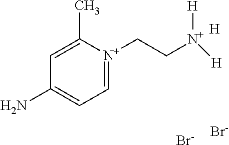

4-Amino-1-[2-(1,3-dioxo-1,3-dihydro-2H-isoindol-2-yl)ethyl]-2-methylpyridinium bromide (970 mg, 2.68 mmol) in 3.5 ml of 48% strength aqueous hydrogen bromide solution was heated under reflux at 100° C. for 30 hours. The reaction mixture was cooled and the solid formed was filtered off and discarded. The filtrate was concentrated and the residue was stirred with tetrahydrofuran. The solid was filtered off, washed with tetrahydrofuran and dried under high vacuum. This gave 900 mg (96% of theory, 89% purity) of the title compound.

LC-MS (Method 5): Rₜ=0.66 min; MS (ESIpos): m/z=152 [M−HBr-Br]⁺

### Example 23A

### 1-[3-(1,3-Dioxo-1,3-dihydro-2H-isoindol-2-yl)propyl]-3-(methylamino)pyridinium bromide

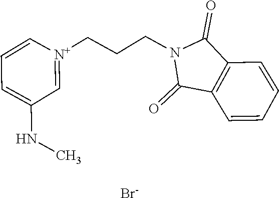 [SMILES: CNc1ccc[n+](CCCN2C(=O)c3ccccc3C2=O)c1 ; [Br-]]

N-Methylpyridine-3-amine (1.00 g, 9.25 mmol) was initially charged in 20 ml of N,N-dimethylformamide, 2-(3-bromopropyl)-1H-isoindole-1,3(2H)-dione (2.48 g, 9.25 mmol) was added and the mixture was stirred at 110° C. overnight. The precipitated solid was filtered off with suction, washed with methyl tert-butyl ether and dried under high vacuum. This gave 2.20 g (63% of theory, 100% purity) of the title compound.

LC-MS (Method 2): Rₜ=0.74 min; MS (ESIpos): m/z=296 [M-Br]⁺

¹H-NMR (400 MHz, DMSO-d6) δ [ppm]: 2.225 (1.01), 2.241 (3.14), 2.259 (4.36), 2.276 (3.23), 2.293 (1.07), 2.772 (16.00), 2.784 (15.76), 3.649 (4.40), 3.664 (8.09), 3.680 (4.14), 4.503 (3.90), 4.522 (6.20), 4.540 (3.71), 7.193 (2.63), 7.205 (2.53), 7.563 (2.65), 7.567 (2.43), 7.585 (3.54), 7.589 (3.28), 7.700 (2.99), 7.714 (3.28), 7.722 (2.39), 7.736 (2.30), 7.846 (3.32), 7.856 (4.85), 7.859 (4.97), 7.868 (10.50), 7.875 (5.84), 7.877 (5.58), 7.884 (10.44), 7.890 (5.19), 7.894 (4.62), 7.896 (4.30), 7.906 (2.93), 8.156 (5.84), 8.188 (4.27), 8.202 (3.96).

### Example 24A

### 1-(3-Ammoniopropyl)-3-(methylamino)pyridinium dibromide

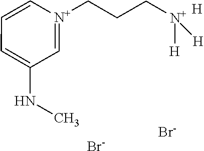

1-[3-(1,3-Dioxo-1,3-dihydro-2H-isoindol-2-yl)propyl]-3-(methylamino)pyridinium bromide (2.20 g, 5.85 mmol) in 7.7 ml of 48% strength aqueous hydrogen bromide solution was heated under reflux at 100° C. for 36 hours. The reaction mixture was cooled and the solid formed was filtered off and discarded. The filtrate was concentrated and the residue was stirred with tetrahydrofuran. The solid was filtered off, washed with tetrahydrofuran and dried under high vacuum. This gave 120 mg (6% of theory) of the title compound.

¹H-NMR (400 MHz, DMSO-d6) δ [ppm]: −0.195 (1.43), 2.444 (0.43), 2.459 (0.95), 2.475 (1.49), 2.490 (0.91), 2.791 (14.20), 3.275 (1.06), 3.291 (1.46), 3.306 (1.00), 4.556 (1.53), 4.571 (2.34), 4.586 (1.48), 7.558 (0.61), 7.561 (0.59), 7.576 (1.01), 7.579 (1.04), 7.616 (0.97), 7.622 (0.41), 7.628 (1.02), 7.634 (0.57), 7.646 (0.57), 7.951 (2.14), 7.960 (2.56), 8.116 (16.00), 10.477 (13.24).

### Example 25A

### 3-{[(tert-Butoxycarbonyl)amino]methyl}-1-methylpyridinium iodide

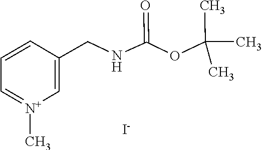 [SMILES: C[n+]1cccc(CNC(=O)OC(C)(C)C)c1 ; [I-]]

In a closed vessel, tert-butyl (pyridin-3-ylmethyl)carbamate (400 mg, 1.92 mmol) and iodomethane (140 μl, 2.3 mmol) in 2.3 ml of acetone were shaken at 75° C. overnight. The reaction mixture was concentrated and the residue was purified by preparative HPLC (column: Chromatorex C18 10 μm, 125×40 mm, mobile phase A=water, B=acetonitrile; gradient: 0.0 min 5% B; 3 min 5% B; 20 min 50% B; 23 min 100% B; 26 min 5% B; flow rate: 50 ml/min; 0.1% formic acid). The product-containing fractions were combined, concentrated and dried under high vacuum. This gave 575 mg (85% of theory, 100% purity) of the title compound.

LC-MS (Method 2): Rₜ=0.52 min; MS (ESIpos): m/z=223 [M-I]⁺

### Example 26A

### 3-(Aminomethyl)-1-methylpyridinium iodide hydrochloride (1:1:1)

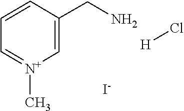

3-{[(tert-Butoxycarbonyl)amino]methyl}-1-methylpyridinium iodide (572 mg, 1.63 mmol) was initially charged in 4 ml of dichloromethane, hydrochloric acid in 1,4-dioxane (4.1 ml, 4.0 M, 16 mmol) was added and the mixture was stirred at room temperature for 1.5 hours. The reaction solution was concentrated and taken up in acetonitrile and re-concentrated three times. The residue was dried under high vacuum. This gave 415 mg (89% of theory, 100% purity) of the title compound.

¹H-NMR (400 MHz, DMSO-d6) δ [ppm]: −0.008 (0.56), 0.008 (0.54), 4.264 (4.19), 4.367 (16.00), 8.196 (1.10), 8.211 (1.33), 8.216 (1.38), 8.231 (1.25), 8.719 (1.55), 8.739 (1.60), 8.783 (1.46), 8.999 (1.66), 9.014 (1.60), 9.229 (2.42).

### Example 27A

### 4-(Dimethylamino)-1-[2-(1,3-dioxo-1,3-dihydro-2H-isoindol-2-yl)ethyl]pyridinium bromide

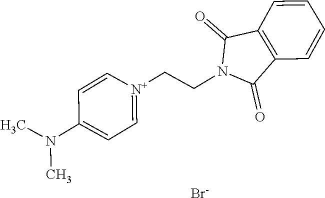 [SMILES: CN(C)c1cc[n+](CCN2C(=O)c3ccccc3C2=O)cc1 ; [Br-]]

N,N-Dimethylpyridine-4-amine (2.00 g, 16.4 mmol) was initially charged in 20 ml of N,N-dimethylformamide, 2-(2-bromoethyl)-1H-isoindole-1,3(2H)-dione (4.16 g, 16.4 mmol) was added and the mixture was stirred at 110° C. overnight. The precipitated solid was filtered off with suction, washed with methyl tert-butyl ether and dried under high vacuum. This gave 5.04 g (82% of theory, 100% purity) of the title compound.

LC-MS (Method 2): Rₜ=0.75 min; MS (ESIpos): m/z=296 [M-Br]⁺

### Example 28A

### 1-(2-Aminoethyl)-4-(dimethylamino)pyridinium bromide hydrobromide (1:1:1)

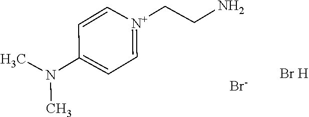

4-(Dimethylamino)-1-[2-(1,3-dioxo-1,3-dihydro-2H-isoindol-2-yl)ethyl]pyridinium bromide (5.04 g, 13.4 mmol) in 19 ml of 48% strength aqueous hydrogen bromide solution was stirred at 100° C. overnight.

The reaction mixture was cooled and the solid formed was filtered off and discarded. The filtrate was concentrated and the residue was stirred with tetrahydrofuran. The solid was filtered off, washed with tetrahydrofuran and dried under high vacuum. This gave 3.55 g (81% of theory, 100% purity) of the title compound.

LC-MS (Method 1): Rₜ=1.32 min; MS (ESIpos): m/z=166 [M−HBr-Br]⁺

¹H-NMR (400 MHz, DMSO-d6) δ [ppm]: 3.040 (0.67), 3.172 (1.20), 3.359 (6.47), 3.388 (2.45), 3.419 (13.71), 4.440 (7.12), 4.455 (12.63), 4.470 (6.81), 7.109 (15.57), 7.128 (16.00), 8.109 (6.23), 8.289 (14.04), 8.308 (13.55).

### Example 29A

### tert-Butyl [(4-methylpyridin-2-yl)methyl]carbamate

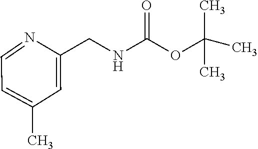 [SMILES: Cc1ccnc(CNC(=O)OC(C)(C)C)c1]

1-(4-Methylpyridin-2-yl)methanamine (250 mg, 2.05 mmol) was initially charged in 22 ml dioxane/water 1/1, and potassium carbonate (2.83 g, 20.5 mmol) and di-tert-butyl dicarbonate (520 μl, 2.3 mmol) were added in succession. The mixture was stirred at room temperature overnight. The phases were separated and the aqueous phase was extracted 2× with ethyl acetate. The combined organic phases were dried over sodium sulfate and filtered, the filtrate was concentrated and the residue was dried under high vacuum. This gave 415 mg (95% pure, 87% of theory) of the title compound.

LC-MS (Method 2): Rₜ=0.85 min; MS (ESIpos): m/z=223 [M+H]⁺

### Example 30A

### 2-{[(tert-Butoxycarbonyl)amino]methyl}-1,4-dimethylpyridinium iodide

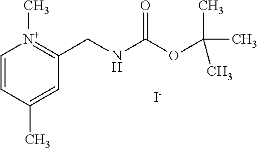 [SMILES: Cc1cc[n+](C)c(CNC(=O)OC(C)(C)C)c1 ; [I-]]

In a closed vessel, tert-butyl [(4-methylpyridin-2-yl)methyl]carbamate (415 mg, 95% pure, 1.77 mmol) and iodomethane (130 μl, 2.1 mmol) in 2.1 ml of acetone were shaken at 75° C. overnight. The reaction solution was concentrated, the residue was concentrated 3× with acetonitrile and dried under high vacuum. This gave 676 mg (97% pure, 102% of theory) of the title compound.

LC-MS (Method 2): Rₜ=0.62 min; MS (ESIpos): m/z=237 [M-I]⁺

### Example 31A

### 2-(Aminomethyl)-1,4-dimethylpyridinium iodide hydrochloride (1:1:1)

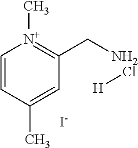

Hydrochloric acid in dioxane (4.6 ml, 4.0 M, 19 mmol) was added to 2-{[(tert-butoxycarbonyl)amino]methyl}-1,4-dimethylpyridinium iodide (676 mg, 1.86 mmol), and the mixture was stirred at room temperature for one hour. The reaction solution was concentrated, and the residue was concentrated three more times with acetonitrile and dried under high vacuum. This gave 428 mg (100% pure, 77% of theory) of the title compound.

LC-MS (Method 1): Rₜ=1.26 min; MS (ESIpos): m/z=137 [M-I-HCl]⁺

### Example 32A

### 1-[2-(1,3-Dioxo-1,3-dihydro-2H-isoindol-2-yl)ethyl]-3-methyl-4-(methylamino)pyridinium chloride

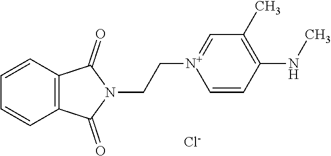 [SMILES: CNc1cc[n+](CCN2C(=O)c3ccccc3C2=O)cc1C ; [Cl-]]

Under argon, N,3-dimethylpyridine-4-amine (689 mg, 5.64 mmol) was initially charged in 12 ml of N,N-dimethylformamide, 2-(2-chlorethyl)-1H-isoindole-1,3(2H)-dione (1.18 g, 5.64 mmol) was added and the mixture was stirred at 110° C. for 48 hours. The precipitated solid was filtered off with suction, washed with diethyl ether and ethyl acetate and dried under high vacuum. This gave 1.14 g (100% pure, 61% of theory) of the title compound.

LC-MS (Method 2): Rₜ=0.74 min; MS (ESIpos): m/z=296 [M-Cl]⁺

¹H-NMR (400 MHz, DMSO-d6) δ [ppm]: 2.059 (16.00), 2.911 (8.46), 2.922 (8.56), 3.998 (2.07), 4.010 (2.97), 4.023 (2.39), 4.348 (2.42), 4.362 (2.95), 4.374 (2.12), 6.790 (2.82), 6.808 (2.88), 7.830 (0.78), 7.834 (0.58), 7.842 (1.63), 7.852 (10.91), 7.857 (11.45), 7.866 (1.61), 7.874 (0.54), 7.879 (0.74), 8.047 (0.97), 8.253 (3.63), 8.300 (1.93), 8.318 (1.86).

### Example 33A

### 1-(2-Aminoethyl)-3-methyl-4-(methylamino)pyridinium chloride hydrochloride (1:1:1)

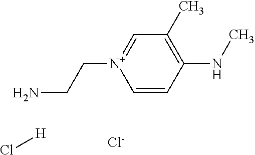

1-[2-(1,3-Dioxo-1,3-dihydro-2H-isoindol-2-yl)ethyl]-3-methyl-4-(methylamino)pyridinium chloride (1.14 g, 3.44 mmol) in 5.5 ml of conc. hydrochloric acid (37% strength in water) was stirred at 100° C. overnight. A further 1.5 ml of conc. hydrochloric acid (37% strength in water) were added and the mixture was once more stirred at 100° C. overnight. On cooling, a solid precipitated. The latter was filtered off with suction and discarded. The filtrate was concentrated and the solid was recrystallized from tetrahydrofuran/acetonitrile. This gave 799 mg (95% pure, 93% of theory) of the title compound.

LC-MS (Method 1): Rₜ=1.30 min; MS (ESIpos): m/z=166 [M-Cl-HCl]⁺

¹H-NMR (400 MHz, DMSO-d6) δ [ppm]: 2.121 (16.00), 2.949 (7.77), 2.961 (7.76), 3.312 (2.76), 3.333 (4.93), 4.481 (2.87), 4.493 (1.53), 6.917 (2.27), 6.935 (2.30), 8.115 (0.93), 8.263 (2.44), 8.352 (1.44), 8.370 (1.39), 8.547 (1.44).

### Example 34A

### 1-[2-(1,3-Dioxo-1,3-dihydro-2H-isoindol-2-yl)ethyl]-2-methyl-4-(methylamino)pyridinium chloride

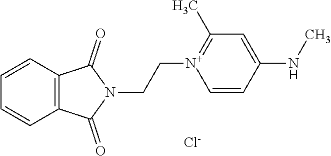 [SMILES: CNc1cc[n+](CCN2C(=O)c3ccccc3C2=O)c(C)c1 ; [Cl-]]

Under argon, N,2-dimethylpyridine-4-amine (895 mg, 7.32 mmol) was initially charged in 15 ml of N,N-dimethylformamide, 2-(2-chlorethyl)-1H-isoindole-1,3(2H)-dione (1.54 g, 7.32 mmol) was added and the mixture was stirred at 110° C. for 48 hours. The precipitated solid was filtered off with suction, washed with diethyl ether and ethyl acetate and dried under high vacuum. The solid was then purified on silica gel (mobile phase: dichloromethane/methanol/formic acid 100/10/1 to 100/30/1). The product fractions were combined and concentrated, and the residue was dried under high vacuum. This gave 313 mg (100% pure, 13% of theory) of the title compound.

LC-MS (Method 3): Rₜ=0.45 min; MS (ESIpos): m/z=296 [M-Cl]⁺

¹H-NMR (400 MHz, DMSO-d6) δ [ppm]: 2.605 (16.00), 2.828 (5.30), 2.840 (5.41), 2.862 (7.91), 2.874 (7.81), 3.943 (3.52), 3.957 (6.36), 3.971 (3.94), 4.370 (2.38), 4.384 (5.06), 4.398 (4.49), 4.412 (1.58), 6.652 (1.30), 6.672 (2.25), 6.685 (1.17), 6.691 (1.14), 6.751 (1.69), 6.841 (2.93), 6.847 (2.78), 7.845 (2.28), 7.856 (3.86), 7.868 (12.91), 7.877 (13.37), 7.888 (3.90), 7.899 (2.22), 7.976 (2.93), 7.994 (2.85), 8.205 (11.61), 8.228 (1.84), 8.712 (1.16).

### Example 35A

### 1-(2-Aminoethyl)-2-methyl-4-(methylamino)pyridinium chloride hydrochloride (1:1:1)

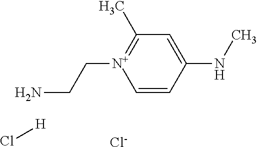

1-[2-(1,3-Dioxo-1,3-dihydro-2H-isoindol-2-yl)ethyl]-2-methyl-4-(methylamino)pyridinium chloride (310 g, 934 μmol) in 1.5 ml of conc. hydrochloric acid (37% strength in water) was stirred at 100° C. overnight. A further 1.5 ml of conc. hydrochloric acid (37% strength in water) were added and the mixture was once more stirred at 100° C. overnight. On cooling, a solid precipitated. The solid was filtered off with suction, washed with water and discarded. The filtrate was concentrated and the residue was recrystallized from tetrahydrofuran/acetonitrile/methanol. This gave 182 mg (90% pure, 74% of theory) of the title compound.

LC-MS (Method 1): Rₜ=0.82 min; MS (ESIpos): m/z=166 [M-Cl-HCl]⁻

¹H-NMR (400 MHz, DMSO-d6) δ [ppm]: −0.008 (1.07), 0.008 (0.91), 2.518 (2.43), 2.591 (2.46), 2.880 (2.68), 2.892 (2.66), 3.229 (0.62), 3.318 (16.00), 3.324 (9.54), 4.392 (0.56), 4.406 (0.75), 4.420 (0.49), 6.847 (0.78).

### Example 36A

### 1-[2-(1,3-Dioxo-1,3-dihydro-2H-isoindol-2-yl)ethyl]-4-(ethylamino)pyridinium chloride

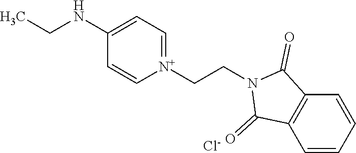 [SMILES: CCNc1cc[n+](CCN2C(=O)c3ccccc3C2=O)cc1 ; [Cl-]]

N-Ethylpyridine-4-amine (500 mg, 4.09 mmol) was initially charged in 10 ml of N,N-dimethylformamide, 2-(2-chloroethyl)-1H-isoindole-1,3(2H)-dione (858 mg, 4.09 mmol) was added and the mixture was stirred at 110° C. over the weekend. The precipitated solid was filtered off with suction, washed with methyl tert-butyl ether and dried under high vacuum. This gave 849 mg (100% pure, 62% of theory) of the title compound.

LC-MS (Method 2): Rₜ=0.77 min; MS (ESIpos): m/z=296 [M-Cl]⁺

### Example 37A

### 1-(2-Ammonioethyl)-4-(ethylamino)pyridinium dichloride

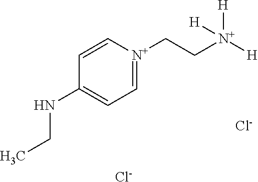

1-[2-(1,3-Dioxo-1,3-dihydro-2H-isoindol-2-yl)ethyl]-4-(ethylamino)pyridinium chloride (848 mg, 2.56 mmol) in 5 ml of conc. hydrochloric acid (37% strength in water) was heated under reflux at 100° C. overnight. On cooling, a solid precipitated. The latter was filtered off and discarded. The filtrate was concentrated and the residue was stirred with tetrahydrofuran. The solid was filtered off, washed with tetrahydrofuran and dried under high vacuum. This gave 543 mg (100% pure, 89% of theory) of the title compound.

¹H-NMR (400 MHz, DMSO-d6) δ [ppm]: −0.008 (2.58), 1.172 (7.58), 1.190 (16.00), 1.208 (7.59), 2.328 (0.56), 2.670 (0.58), 3.283 (3.50), 3.301 (6.90), 3.332 (5.53), 3.351 (1.33), 4.407 (2.71), 4.420 (3.54), 6.891 (1.65), 6.898 (1.67), 6.909 (1.74), 6.958 (1.93), 6.976 (1.97), 8.130 (1.86), 8.145 (1.52), 8.300 (2.65), 8.317 (2.71), 8.859 (1.14).

### Example 38A

### 1-[2-(1,3-Dioxo-1,3-dihydro-2H-isoindol-2-yl)ethyl]-3-ethylpyridinium bromide

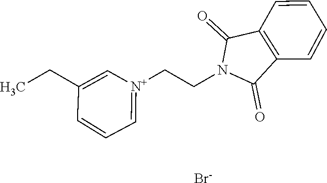 [SMILES: CCc1ccc[n+](CCN2C(=O)c3ccccc3C2=O)c1 ; [Br-]]

3-Ethylpyridine (2.00 g, 18.7 mmol) was initially charged in 20 ml of N,N-dimethylformamide, 2-(2-bromoethyl)-1H-isoindole-1,3(2H)-dione (4.74 g, 18.7 mmol) was added and the mixture was stirred at 110° C. overnight. The N,N-dimethylformamide was removed on a rotary evaporator and the residue was stirred with methyl tert-butyl ether. The precipitated solid was filtered off with suction, washed with methyl tert-butyl ether and dried under high vacuum. This gave 5.30 g (100% pure, 79% of theory) of the title compound.

LC-MS (Method 2): Rₜ=0.70 min; MS (ESIpos): m/z=282 [M-Br]⁺

### Example 39A

### 1-(2-Aminoethyl)-3-ethylpyridinium bromide hydrobromide (1:1:1)

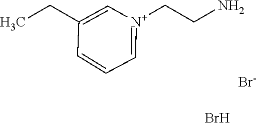

1-[2-(1,3-Dioxo-1,3-dihydro-2H-isoindol-2-yl)ethyl]-3-ethylpyridinium bromide (5.30 g, 14.7 mmol) in 20 ml of conc. hydrobromic acid (48% strength in water) was heated under reflux at 100° C. overnight. On cooling, a solid precipitated. The latter was filtered off and discarded. The filtrate was concentrated and the residue was stirred with tetrahydrofuran. The solid was filtered off, washed with tetrahydrofuran and dried under high vacuum. This gave 3.61 g (79% of theory) of the title compound.

¹H-NMR (400 MHz, DMSO-d6) δ [ppm]: 1.274 (7.42), 1.293 (16.00), 1.312 (7.71), 2.820 (1.89), 2.839 (5.59), 2.858 (5.48), 2.877 (1.77), 3.383 (4.56), 4.861 (2.70), 4.875 (4.27), 4.890 (2.55), 8.127 (3.08), 8.143 (4.28), 8.147 (4.58), 8.162 (4.07), 8.545 (2.44), 8.565 (2.23), 8.924 (2.40), 8.939 (2.31), 9.056 (3.59).

### Example 40A

### 2-{[(tert-Butoxycarbonyl)amino]methyl}-1-methylpyridinium iodide

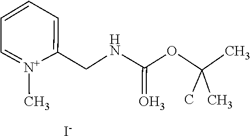

In a closed vessel, tert-butyl (pyridin-2-ylmethyl)carbamate (470 μl, 2.4 mmol) and iodomethane (180 μl, 2.9 mmol) in 2.5 ml of acetone were stirred at 75° C. overnight. The reaction mixture was concentrated and the residue was dried under high vacuum. This gave 810 mg (96% of theory) of the title compound.

¹H-NMR (400 MHz, DMSO-d6) δ [ppm]: −0.008 (0.87), 0.008 (0.49), 1.366 (0.77), 1.425 (16.00), 1.487 (0.99), 2.086 (7.32), 2.519 (0.50), 4.290 (12.40), 4.583 (2.42), 4.597 (2.27), 7.887 (0.89), 7.898 (1.23), 7.918 (0.92), 7.988 (0.61), 8.006 (1.06), 8.022 (0.62), 8.551 (0.64), 8.570 (1.10), 8.590 (0.54), 8.972 (1.38), 8.987 (1.31).

### Example 41A

### 2-(Aminomethyl)-1-methylpyridinium iodide hydrochloride (1:1:1)

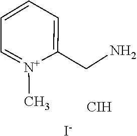

2-{[(tert-butoxycarbonyl)amino]methyl}-1-methylpyridinium iodide (810 mg, 2.31 mmol) was initially charged in 24 ml of dichloromethane, hydrochloric acid in dioxane (5.8 ml, 4.0 M, 23 mmol) was added and the mixture was stirred at room temperature overnight. The reaction mixture was concentrated and the residue was stirred with dichloromethane. The precipitated solid was filtered off with suction, washed with dichloromethane and dried under high vacuum. This gave 627 mg (100% pure, 95% of theory) of the title compound.

LC-MS (Method 1): R&=0.25 min; MS (ESIpos): m/z=123 [M-I-HCl]⁺

### Example 42A

### 1-[2-(1,3-Dioxo-1,3-dihydro-2H-isoindol-2-yl)ethyl]-4-(trifluoromethyl)pyridinium bromide

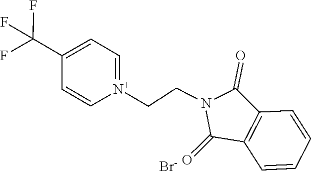 [SMILES: O=C1c2ccccc2C(=O)N1CC[n+]1ccc(C(F)(F)F)cc1 ; [Br-]]

4-(Trifluoromethyl)pyridine (310 μl, 2.7 mmol) was initially charged in 10 ml of N,N-dimethylformamide, 2-(2-bromoethyl)-1H-isoindole-1,3(2H)-dione (677 mg, 2.66 mmol) was added and the mixture was stirred at 110° C. for 72 hours. The reaction mixture was concentrated, methyl tert-butyl ether was added to the oily residue and the mixture was concentrated again. The residue, which was now solid, was stirred with methyl tert-butyl ether, and the solid was filtered off, washed with methyl tert-butyl ether and dried under high vacuum. This gave 390 mg (100% pure, 36% of theory) of the title compound.

LC-MS (Method 3): Rₜ=0.45 min; MS (ESIpos): m/z=321 [M-Br]⁺

### Example 43A

### 1-(2-Ammonioethyl)-4-(trifluoromethyl)pyridinium dibromide

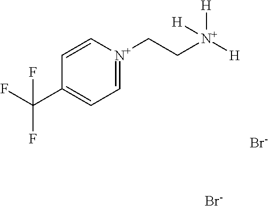 [SMILES: [NH3+]CC[n+]1ccc(C(F)(F)F)cc1 ; [Br-] ; [Br-]]

1-[2-(1,3-Dioxo-1,3-dihydro-2H-isoindol-2-yl)ethyl]-4-(trifluoromethyl)pyridinium bromide (390 mg, 972 μmol) in 5 ml of conc. hydrobromic acid (48% strength in water) was heated under reflux at 100° C. overnight. On cooling, a solid precipitated. The latter was filtered off and discarded. The filtrate was concentrated. The residue was stirred with tetrahydrofuran, and the solid was filtered off, washed with tetrahydrofuran and dried under high vacuum. This gave 181 mg (100% pure, 53% of theory) of the title compound.

¹H-NMR (400 MHz, DMSO-d6) δ [ppm]: 2.329 (0.77), 2.671 (0.69), 3.584 (9.46), 5.002 (10.26), 8.043 (6.71), 8.789 (15.09), 8.805 (16.00), 9.428 (11.27), 9.442 (10.28).

### Example 44A

### tert-Butyl [(5-methylpyridin-2-yl)methyl]carbamate

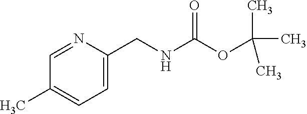 [SMILES: Cc1ccc(CNC(=O)OC(C)(C)C)nc1]

1-(5-Methylpyridin-2-yl)methanamine (150 mg, 1.23 mmol) was initially charged in 4.1 ml of sodium hydroxide solution (1N in water), di-tert-butyl dicarbonate (340 μl, 1.5 mmol) was added at 0° C. and the mixture was stirred at room temperature overnight. Ethyl acetate was added to the reaction mixture and the mixture was washed 2× with water and 1× with saturated NaCl solution. The organic phase was dried over sodium sulfate, filtered and concentrated. The residue was purified by column chromatography (10 g Biotage Snap Cartridge Ultra®; Biotage-Isolera-One®; CH/EA gradient, TLC: CH/EA 1/1). The product fractions were combined and concentrated, and the residue was dried under high vacuum. This gave 168 mg (100% pure, 62% of theory) of the title compound.

LC-MS (Method 3): Rₜ=0.50 min; MS (ESIpos): m/z=223 [M+H]⁺

### Example 45A

### 2-{[(tert-Butoxycarbonyl)amino]methyl}-1,5-dimethylpyridinium iodide

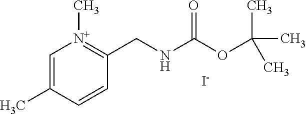 [SMILES: Cc1ccc(CNC(=O)OC(C)(C)C)[n+](C)c1 ; [I-]]

In a closed vessel, tert-butyl [(5-methylpyridin-2-yl)methyl]carbamate (372 mg, 78% pure, 1.31 mmol) and iodomethane (98 μl, 1.6 mmol) in 1.6 ml of acetone were shaken at 75° C. overnight. The reaction solution was concentrated, the residue was concentrated 3× from acetonitrile and dried under high vacuum.

This gave 597 mg (98% pure, 123% of theory) of the title compound.

LC-MS (Method 2): Rₜ=0.66 min; MS (ESIpos): m/z=237 [M-I]⁺

### Example 46A

### 2-(Aminomethyl)-1,5-dimethylpyridinium iodide hydrochloride (1:1:1)

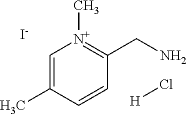

Hydrochloric acid in dioxane (4.1 ml, 4.0 M, 16 mmol) was added to 2-{[(tert-butoxycarbonyl)amino]methyl}-1,5-dimethylpyridinium iodide (597 mg, 1.64 mmol) and the mixture was stirred at room temperature for one hour. The reaction solution was concentrated, the residue was concentrated 3× from acetonitrile and dried under high vacuum. This gave 483 mg (95% pure, 93% of theory) of the title compound.

LC-MS (Method 1): Rₜ=1.67 min; MS (ESIpos): m/z=137 [M-I-HCl]⁺

### Example 47A

### 1-[2-(1,3-Dioxo-1,3-dihydro-2H-isoindol-2-yl)ethyl]-3-methylpyridinium bromide

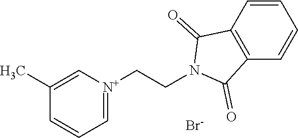 [SMILES: Cc1ccc[n+](CCN2C(=O)c3ccccc3C2=O)c1 ; [Br-]]

3-Methylpyridine (2.00 g, 21.5 mmol) was initially charged in 20 ml of N,N-dimethylformamide, 2-(2-bromoethyl)-1H-isoindole-1,3(2H)-dione (5.46 g, 21.5 mmol) was added and the mixture was stirred at 110° C. overnight. The N,N-dimethylformamide was removed on a rotary evaporator and the residue was stirred with methyl tert-butyl ether. The precipitated solid was filtered off with suction, washed with methyl tert-butyl ether and dried under high vacuum. This gave 6.39 g (96% pure, 82% of theory) of the title compound.

LC-MS (Method 2): Rₜ=0.60 min; MS (ESIpos): m/z=268 [M-Br]⁺

### Example 48A

### 1-(2-Aminoethyl)-3-methylpyridinium bromide hydrobromide (1:1:1)

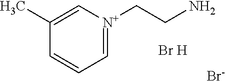

1-[2-(1,3-Dioxo-1,3-dihydro-2H-isoindol-2-yl)ethyl]-3-methylpyridinium bromide (6.39 g, 18.4 mmol) in 25 ml of conc. hydrobromic acid (48% strength in water) was heated under reflux at 100° C. overnight.

On cooling, a solid precipitated. The latter was filtered off and discarded. The filtrate was concentrated and the residue was stirred with tetrahydrofuran. The precipitated solid was filtered off, washed with tetrahydrofuran and dried under high vacuum. This gave 4.55 g (83% of theory) of the title compound.

¹H-NMR (400 MHz, DMSO-d6) δ [ppm]: 2.518 (16.00), 3.544 (1.90), 4.829 (1.79), 4.843 (3.01), 4.857 (1.68), 8.101 (2.52), 8.117 (3.09), 8.121 (3.19), 8.136 (2.60), 8.494 (1.72), 8.514 (1.56), 8.894 (1.57), 8.909 (1.50), 9.016 (2.34).

### Example 49A

### tert-Butyl [3-(4-methyl-1H-pyrazol-1-yl)propyl]carbamate

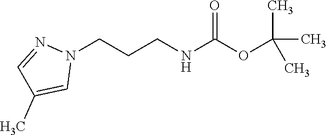 [SMILES: Cc1cnn(CCCNC(=O)OC(C)(C)C)c1]

3-(4-Methyl-1H-pyrazol-1-yl)propane-1-amine (350 mg, 2.51 mmol) was initially charged in 10 ml of tetrahydrofuran and cooled to 0° C. At this temperature, triethylamine (1.1 ml, 7.5 mmol), 4-dimethylaminopyridine (46.1 mg, 377 μmol) and di-tert-butyl dicarbonate (610 μl, 2.6 mmol) were added in succession. The reaction mixture was then allowed to slowly warm to room temperature and stirred overnight. The reaction mixture was partitioned between water and ethyl acetate, the organic phase was washed with water and with saturated NaCl solution, dried ober sodium sulfate and filtered, the filtrate was concentrated and the residue was dried under high vacuum. This gave 537 mg (43% pure, 39% of theory) of the title compound.

LC-MS (Method 6): Rₜ=2.18 min; MS (ESIpos): m/z=240 [M+H]⁺

### Example 50A

### 1-{3-[(tert-Butoxycarbonyl)amino]propyl}-2,4-dimethyl-1H-pyrazol-2-ium formate

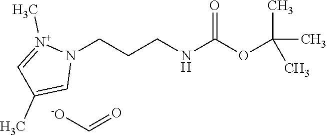

In a closed vessel, tert-butyl [3-(4-methyl-1H-pyrazol-1-yl)propyl]carbamate (537 mg, 2.24 mmol; 43% pure) and iodomethane (170 μl, 2.7 mmol) in 2.7 ml of acetone were shaken at 75° C. overnight. The reaction solution was concentrated and the residue was purified by preparative HPLC (column: Chromatorex C18 10 μm, 125×40 mm, mobile phase A=water, B=acetonitrile; gradient: 0.0 min 5% B; 3 min 5% B; 20 min 50% B; 23 min 100% B; 26 min 5% B; flow rate: 50 ml/min; 0.1% formic acid). The product fractions were combined and concentrated, and the residue was dried under high vacuum. This gave 403 mg (70% pure, 33% of theory) of the title compound.

LC-MS (Method 3): Rₜ=0.41 min; MS (ESIpos): m/z=254 [M-I]⁺

### Example 51A

### 1-(3-Aminopropyl)-2,4-dimethyl-1H-pyrazol-2-ium formate hydrochloride (1:1:1)

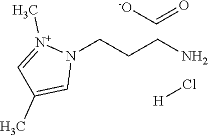

Hydrochloric acid in dioxane (2.6 ml, 4.0 M, 11 mmol) was added to 1-{3-[(tert-butoxycarbonyl)amino]propyl}-2,4-dimethyl-1H-pyrazol-2-ium formate (403 mg, 1.06 mmol), and the mixture was stirred at room temperature for 3.5 hours. The reaction solution was concentrated, and the residue was concentrated three more times from acetonitrile and dried under high vacuum. This gave 331 mg (98% pure, 97% of theory) of the title compound.

LC-MS (Method 2): Rₜ=0.24 min; MS (ESIpos): m/z=154 [M-I-HCl]⁺

### Example 52A

### Methyl 3-(1-isopropyl-1H-pyrazol-5-yl)imidazo[1,2-a]pyridine-7-carboxylate

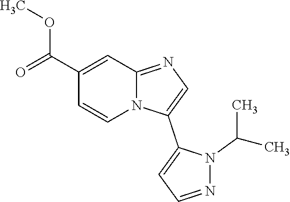 [SMILES: COC(=O)c1ccn2c(-c3ccnn3C(C)C)cnc2c1]

Under argon, methyl 3-iodoimidazo[1,2-a]pyridine-7-carboxylate (200 mg, 662 μmol), (1-isopropyl-1H-pyrazol-5-yl)boric acid (122 mg, 795 μmol) and potassium carbonate (302 mg, 2.2 mmol) were initially charged in 4 ml of dioxane, and the mixture was degassed with argon for 10 minutes. [1,1-Bis(diphenylphosphino)ferrocene]dichloropalladium/dichloromethane complex (27.0 mg, 33.1 μmol) was then added and the reaction mixture was stirred at 110° C. overnight. The mixture was concentrated and the residue was taken up in ethyl acetate and washed with water and saturated NaCl solution. The organic phase was dried over sodium sulfate, filtered and concentrated. The residue was purified by column chromatography (10 g Biotage Snap Cartridge Ultra®; Biotage-Isolera-One®; CH/EA gradient, 12% EA-100% EA; flow rate: 36 ml/min; TLC: CH/EA 1/1). The product fractions were concentrated and the residue was dried under high vacuum. This gave 80 mg (89% pure, 38% of theory) of the title compound.

LC-MS (Method 2): Rₜ=1.36 min; MS (ESIpos): m/z=285 [M+H]⁺

### Example 53A

### Lithium 3-(1-isopropyl-1H-pyrazol-5-yl)imidazo[1,2-a]pyridine-7-carboxylate

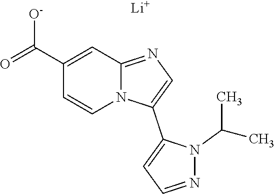 [SMILES: [Li+] ; CC(C)n1nccc1-c1cnc2cc(C(=O)[O-])ccn12]

Methyl 3-(1-isopropyl-1H-pyrazol-5-yl)imidazo[1,2-a]pyridine-7-carboxylate (80.0 mg, 89% pure, 250 μmol) was initially charged in 5 ml of tetrahydrofuran/water 3:1, lithium hydroxide (12.0 mg, 500 μmol) was added and the mixture was stirred at 60° C. for 1.5 h. The reaction mixture was concentrated, and the residue was dissolved in acetonitrile/water and lyophilized. This gave 90 mg (100% pure, 130% of theory) of the title compound.

¹H-NMR (400 MHz, DMSO-d6) δ [ppm]: 1.356 (15.82), 1.373 (16.00), 4.352 (0.43), 4.368 (1.10), 4.385 (1.48), 4.401 (1.09), 4.418 (0.43), 6.606 (3.63), 6.611 (3.85), 7.449 (1.57), 7.468 (1.66), 7.674 (0.65), 7.700 (3.24), 7.704 (3.36), 7.765 (4.34), 7.988 (3.46), 8.004 (1.93).

### Example 54A

### 1-[2-(1,3-Dioxo-1,3-dihydro-2H-isoindol-2-yl)ethyl]-4-(methylamino)pyridinium chloride

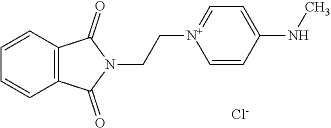 [SMILES: CNc1cc[n+](CCN2C(=O)c3ccccc3C2=O)cc1 ; [Cl-]]

N-Methylpyridine-4-amine (1.00 g, 9.25 mmol) was initially charged in 10 ml of N,N-dimethylformamide, 2-(2-chloroethyl)-1H-isoindole-1,3(2H)-dione (1.94 g, 9.25 mmol) was added and the mixture was stirred at 110° C. overnight. The precipitated solid was filtered off with suction, washed with methyl tert-butyl ether and dried under high vacuum. This gave 1.99 g (100% pure, 68% of theory) of the title compound.

LC-MS (Method 2): Rₜ=0.67 min; MS (ESIpos): m/z=282 [M-Cl-]⁺

¹H-NMR (500 MHz, DMSO-d6) δ [ppm]: 2.857 (16.00), 2.867 (15.78), 3.981 (3.82), 3.991 (4.85), 4.002 (3.96), 4.357 (4.07), 4.368 (4.70), 4.378 (3.51), 6.801 (2.60), 6.806 (2.94), 6.816 (2.64), 6.821 (2.88), 6.882 (3.22), 6.888 (2.82), 6.897 (3.19), 6.903 (2.74), 7.835 (2.94), 7.840 (2.53), 7.844 (4.04), 7.848 (5.05), 7.853 (12.68), 7.861 (12.85), 7.866 (5.22), 7.871 (3.90), 7.874 (2.34), 7.879 (2.71), 8.141 (3.12), 8.145 (3.18), 8.156 (3.02), 8.160 (3.03), 8.350 (2.91), 8.353 (2.83), 8.365 (2.82), 8.368 (2.70), 9.077 (2.07), 9.087 (2.03).

### Example 55A

### 1-(2-Aminoethyl)-4-(methylamino)pyridinium chloride hydrochloride (1:1:1)

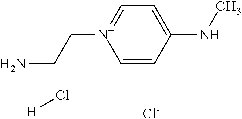

1-[2-(1,3-Dioxo-1,3-dihydro-2H-isoindol-2-yl)ethyl]-4-(methylamino)pyridinium chloride (16.0 g, 50.2 mmol) was stirred in 100 ml of conc. hydrochloric acid at 100° C. for 2 days. The precipitated solid was filtered off with suction and discarded. The filtrate was concentrated and the residue was stirred with tetrahydrofuran. The solid was filtered off with suction, washed with tetrahydrofuran and dried under high vacuum. This gave 12 g (100% pure, 106% of theory) of the title compound.

¹H-NMR (400 MHz, DMSO-d6) δ [ppm]: 2.892 (15.95), 2.904 (16.00), 3.286 (3.87), 3.299 (3.90), 4.462 (4.14), 4.477 (7.20), 4.491 (3.94), 6.910 (2.46), 6.917 (3.11), 6.928 (2.52), 6.935 (3.26), 6.962 (2.92), 6.968 (2.48), 6.980 (2.92), 6.986 (2.51), 8.188 (3.45), 8.206 (3.36), 8.384 (3.46), 8.402 (3.34), 8.575 (3.87), 9.081 (1.81).

### Example 56A

### 3-Iodoimidazo[1,2-a]pyridine-7-carboxylicacid

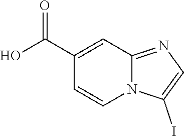 [SMILES: O=C(O)c1ccn2c(I)cnc2c1]

Methyl 3-iodoimidazo[1,2-a]pyridine-7-carboxylate (1.00 g, 3.31 mmol) was initially charged in 20 ml of tetrahydrofuran, lithium hydroxide solution (6.6 ml, 1.0 M, 6.6 mmol) was added and the mixture was stirred at room temperature for 2 hours. The tetrahydrofuran was removed on a rotary evaporator and the aqueous residue was acidified (pH 3) with 4 N hydrochloric acid. The precipitated solid was filtered off with suction, washed with acetonitrile and dried under high vacuum. More solid precipitated from the filtrate. The solid was filtered off with suction, washed with acetonitrile and dried under high vacuum. This gave a total of 743 mg (100% pure, 78% of theory) of the title compound.

LC-MS (Method 2): Rₜ=0.67 min; MS (ESIpos): m/z=288 [M+H]⁺

### Example 57A

### 2-({[(3-Iodoimidazo[1,2-a]pyridin-7-yl)carbonyl]amino}methyl)-1-methylimidazo[1,2-a]pyridin-1-ium iodide

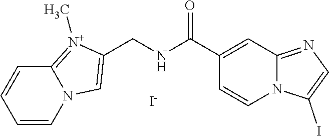 [SMILES: C[n+]1c(CNC(=O)c2ccn3c(I)cnc3c2)cn2ccccc21 ; [I-]]

3-Iodoimidazo[1,2-a]pyridine-7-carboxylic acid (81.4 mg, 283 μmol) was initially charged in 2 ml of dichloromethane, 2-(aminomethyl)-1-methylimidazo[1,2-a]pyridin-1-ium iodide hydrochloride (1:1:1) (92.0 mg, 283 μmol), 1-(3-dimethylaminopropyl)-3-ethylcarbodiimide hydrochloride (81.3 mg, 424 μmol) and 4-dimethylaminopyridine (104 mg, 848 μmol) were added and the mixture was stirred at room temperature overnight. The precipitated solid was filtered off with suction, washed with dichloromethane and dried under high vacuum. This gave 121 mg (100% pure, 99% of theory) of the title compound.

LC-MS (Method 2): Rₜ=0.71 min; MS (ESIpos): m/z=432 [M-I]⁺

### Example 58A

### 4-tert-Butyl-1-[2-(1,3-dioxo-1,3-dihydro-2H-isoindol-2-yl)ethyl]pyridinium bromide

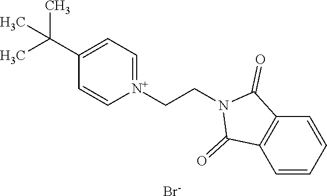 [SMILES: CC(C)(C)c1cc[n+](CCN2C(=O)c3ccccc3C2=O)cc1 ; [Br-]]

4-tert-Butylpyridine (540 μl, 3.7 mmol) was initially charged in 5 ml of N,N-dimethylformamide, 2-(2-bromoethyl)-1H-isoindole-1,3(2H)-dione (940 mg, 3.70 mmol) was added and the mixture was stirred at 110° C. overnight. The reaction mixture was concentrated on a rotary evaporator, methyl tert-butyl ether was added to the residue and the mixture was concentrated again. The residue was dried under high vacuum for 48 hours and then once more stirred with methyl tert-butyl ether and concentrated. The residue was finally stirred with tetrahydrofuran, and the solid was filtered off with suction, washed with tetrahydrofuran and dried under high vacuum. This gave 1.06 g (100% pure, 74% of theory) of the title compound.

LC-MS (Method 2): Rₜ=0.95 min; MS (ESIpos): m/z=309 [M-Br]⁺

### Example 59A

### 1-(2-Ammonioethyl)-4-tert-butylpyridinium dibromide

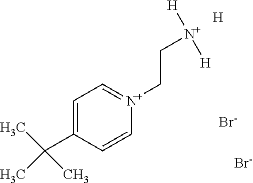

4-tert-Butyl-1-[2-(1,3-dioxo-1,3-dihydro-2H-isoindol-2-yl)ethyl]pyridiniumbromide (1.06 g, 2.72 mmol) in 14 ml of conc. hydrobromic acid (48% strength in water) was stirred at 100° C. for 48 hours. On cooling, a solid precipitated. The solid was filtered off and discarded and the filtrate was concentrated. The residue was stirred with tetrahydrofuran, and the solid was filtered off with suction, washed with tetrahydrofuran and dried under high vacuum. This gave 799 mg (100% pure, 86% of theory) of the title compound.

LC-MS (Method 3): Rₜ=0.14 min; MS (ESIpos): m/z=179 [M−H-2Br]⁺

¹H-NMR (400 MHz, DMSO-d6) δ [ppm]: 1.381 (16.00), 3.522 (0.54), 4.808 (0.43), 4.823 (0.66), 4.837 (0.40), 8.075 (0.41), 8.241 (1.13), 8.258 (1.18), 8.956 (0.88), 8.973 (0.81).

### Example 60A

### 1-[2-(1,3-Dioxo-1,3-dihydro-2H-isoindol-2-yl)ethyl]-4-isopropylpyridinium bromide

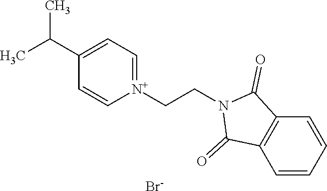 [SMILES: CC(C)c1cc[n+](CCN2C(=O)c3ccccc3C2=O)cc1 ; [Br-]]

4-Isopropylpyridine (500 mg, 4.13 mmol) was initially charged in 5.5 ml of N,N-dimethylformamide, 2-(2-bromoethyl)-1H-isoindole-1,3(2H)-dione (1.05 g, 4.13 mmol) was added and the mixture was stirred at 110° C. overnight. The reaction mixture was concentrated on a rotary evaporator, and the residue was stirred with methyl tert-butyl ether, filtered and dried under high vacuum. This gave 1.28 g (93% pure, 77% of theory) of the title compound.

LC-MS (Method 2): Rₜ=0.83 min; MS (ESIpos): m/z=295 [M-Br]⁺

### Example 61A

### 1-(2-Ammonioethyl)-4-isopropylpyridinium dibromide

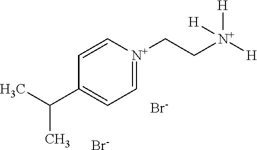

1-[2-(1,3-Dioxo-1,3-dihydro-2H-isoindol-2-yl)ethyl]-4-isopropylpyridinium bromide (1.28 g, 3.41 mmol) was initially charged in 18 ml of conc. hydrobromic acid (48% strength in water) and stirred at 100° C. for 48 hours. On cooling, a solid precipitated. The solid was filtered off and discarded and the filtrate was concentrated. The residue was stirred with tetrahydrofuran, filtered, washed with tetrahydrofuran and dried under high vacuum. This gave 920 mg (95% pure, 79% of theory) of the title compound.

LC-MS (Method 3): Rₜ=0.14 min; MS (ESIpos): m/z=166 [M−HBr-Br]⁺

¹H-NMR (400 MHz, DMSO-d6) δ [ppm]: −0.008 (0.43), 1.288 (15.93), 1.305 (16.00), 3.220 (0.92), 3.237 (1.20), 3.254 (0.88), 3.376 (1.32), 4.793 (1.41), 4.807 (2.21), 4.822 (1.33), 8.070 (1.24), 8.135 (3.54), 8.152 (3.67), 8.943 (2.76), 8.959 (2.56).

### Example 62A

### 1-(2-{[(3-Iodoimidazo[1,2-a]pyridin-7-yl)carbonyl]amino}ethyl)-4-(methylamino)pyridinium bromide

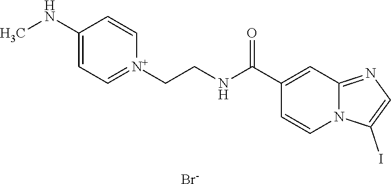 [SMILES: CNc1cc[n+](CCNC(=O)c2ccn3c(I)cnc3c2)cc1 ; [Br-]]

3-Iodoimidazo[1,2-a]pyridine-7-carboxylic acid (550 mg, 1.91 mmol) and 1-(2-aminoethyl)-4-(methylamino)pyridinium bromide hydrobromide (1:1:1) (657 mg, 2.10 mmol) were initially charged in 30 ml of dichloromethane, 1-(3-dimethylaminopropyl)-3-ethylcarbodiimide hydrochloride (549 mg, 2.86 mmol) and 4-dimethylaminopyridine (700 mg, 5.73 mmol) were added and the mixture was stirred at room temperature overnight. The reaction mixture was applied to Isolute® and purified by silica gel chromatography (28 g Snap Cartridge KP-NH Biotage®; Biotage-Isolera-One®; dichloromethane/methanol gradient 10% methanol to 40% methanol). The product-containing fractions were combined, concentrated and dried under high vacuum. This gave 797 mg (95% pure, 79% of theory) of the title compound.

LC-MS (Method 2): Rₜ=0.68 min; MS (ESIpos): m/z=422 [M-Br]⁺

¹H-NMR (400 MHz, DMSO-d6) δ [ppm]: 0.950 (1.15), 0.968 (2.48), 0.986 (1.39), 1.116 (0.45), 1.453 (0.54), 1.470 (0.82), 1.488 (0.58), 2.107 (7.82), 2.136 (0.45), 2.163 (0.59), 2.181 (0.96), 2.199 (0.50), 2.861 (15.62), 2.873 (16.00), 2.943 (10.33), 2.965 (1.51), 2.982 (1.52), 2.997 (0.96), 3.040 (1.42), 3.122 (0.68), 3.162 (1.20), 3.176 (1.24), 3.226 (2.11), 3.706 (4.43), 3.718 (4.65), 3.732 (2.16), 4.304 (3.83), 4.318 (5.58), 4.330 (3.64), 5.756 (0.75), 6.571 (0.92), 6.574 (0.83), 6.587 (1.01), 6.810 (2.49), 6.817 (3.46), 6.828 (2.44), 6.835 (3.81), 6.848 (3.50), 6.854 (2.40), 6.866 (3.32), 6.873 (2.57), 7.361 (3.69), 7.364 (4.05), 7.379 (3.74), 7.382 (4.20), 7.872 (11.63), 8.081 (8.11), 8.106 (3.63), 8.293 (3.37), 8.310 (3.37), 8.395 (5.54), 8.413 (5.27), 8.599 (2.60), 8.611 (2.48), 8.852 (1.67), 8.866 (3.40), 8.881 (1.72).

### Example 63A

### 1-[2-(1,3-Dioxo-1,3-dihydro-2H-isoindol-2-yl)ethyl]-4-ethylpyridinium bromide

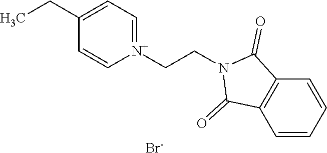 [SMILES: CCc1cc[n+](CCN2C(=O)c3ccccc3C2=O)cc1 ; [Br-]]

4-Ethylpyridine (530 μl, 4.7 mmol) was initially charged in 6 ml of N,N-dimethylformamide, 2-(2-bromoethyl)-1H-isoindole-1,3(2H)-dione (1.19 g, 4.67 mmol) was added and the mixture was stirred at 110° C. overnight. The reaction mixture was concentrated on a rotary evaporator and the residue was stirred with methyl tert-butyl ether. The solid was filtered off with suction, washed with methyl tert-butyl ether and dried under high vacuum. This gave 1.35 g (85% pure, 68% of theory) of the title compound.

LC-MS (Method 3): Rₜ=0.44 min; MS (ESIpos): m/z=281 [M-Br]⁺

### Example 64A

### 1-(2-Ammonioethyl)-4-ethylpyridinium dibromide

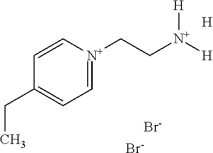

1-[2-(1,3-Dioxo-1,3-dihydro-2H-isoindol-2-yl)ethyl]-4-ethylpyridinium bromide (1.35 g, 3.74 mmol) in 21 ml of conc. hydrobromic acid (48% strength in water) was stirred at 100° C. for 48 hours. On cooling, a solid precipitated. The solid was filtered off and the filtrate was concentrated on a rotary evaporator. The residue was stirred with tetrahydrofuran, and the solid was filtered off with suction, washed with tetrahydrofuran and dried under high vacuum. This gave 1.11 g (95% of theory) of the title compound.

¹H-NMR (400 MHz, DMSO-d6) δ [ppm]: −0.008 (1.05), 0.008 (1.05), 1.262 (7.42), 1.281 (16.00), 1.300 (7.52), 2.731 (7.67), 2.772 (0.63), 2.891 (9.39), 2.907 (1.82), 2.926 (5.16), 2.945 (5.01), 2.964 (1.57), 3.412 (0.54), 3.485 (1.27), 3.500 (2.39), 3.514 (2.45), 3.526 (1.50), 3.542 (0.93), 3.560 (0.43), 3.637 (0.49), 3.652 (0.87), 3.668 (0.43), 3.971 (1.82), 4.782 (2.02), 4.797 (3.46), 4.810 (1.96), 7.953 (1.42), 8.049 (1.89), 8.089 (5.82), 8.106 (5.78), 8.914 (3.80), 8.930 (3.70).

### Example 65A

### 1-[2-(1,3-Dioxo-1,3-dihydro-2H-isoindol-2-yl)ethyl]-3-phenoxypyridinium bromide

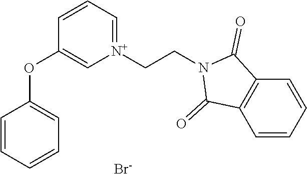 [SMILES: O=C1c2ccccc2C(=O)N1CC[n+]1cccc(Oc2ccccc2)c1 ; [Br-]]

3-Phenoxypyridine (500 mg, 2.92 mmol) was initially charged in 3.8 ml of N,N-dimethylformamide, 2-(2-bromoethyl)-1H-isoindole-1,3(2H)-dione (742 mg, 2.92 mmol) was added and the mixture was stirred at 110° C. for 48 hours. The reaction mixture was concentrated on a rotary evaporator and the residue was stirred with methyl tert-butyl ether. The methyl tert-butyl ether was decanted off and the residue was dried under high vacuum. This gave 1.1 g (78% pure, 69% of theory) of the title compound.

LC-MS (Method 2): Rₜ=0.95 min; MS (ESIpos): m/z=345 [M-Br]⁺

¹H-NMR (400 MHz, DMSO-d6) δ [ppm]: 1.106 (0.80), 2.328 (0.49), 2.670 (0.44), 2.731 (1.84), 2.766 (0.46), 2.778 (0.44), 2.891 (2.16), 3.853 (0.74), 3.866 (1.03), 3.880 (0.59), 3.932 (0.49), 4.153 (2.44), 4.165 (3.19), 4.177 (2.57), 4.311 (0.54), 4.325 (0.93), 4.338 (0.55), 4.819 (2.60), 4.831 (3.18), 4.843 (2.32), 7.064 (0.70), 7.083 (0.80), 7.107 (4.54), 7.126 (5.46), 7.129 (4.33), 7.197 (0.44), 7.258 (1.22), 7.276 (2.98), 7.295 (1.95), 7.395 (3.84), 7.416 (4.99), 7.434 (3.42), 7.450 (0.48), 7.831 (1.77), 7.848 (1.63), 7.857 (1.39), 7.863 (2.38), 7.866 (2.64), 7.878 (16.00), 7.903 (1.24), 8.115 (1.83), 8.129 (1.93), 8.136 (2.26), 8.151 (2.24), 8.165 (0.97), 8.313 (1.99), 8.318 (1.92), 8.335 (1.56), 8.340 (1.62), 8.371 (0.60), 8.382 (0.74), 8.992 (2.61), 9.008 (2.49), 9.215 (3.17).

### Example 66A

### 1-(2-Aminoethyl)-3-phenoxypyridinium bromide

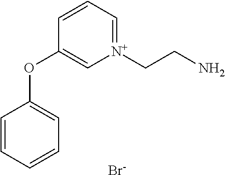 [SMILES: NCC[n+]1cccc(Oc2ccccc2)c1 ; [Br-]]

1-[2-(1,3-Dioxo-1,3-dihydro-2H-isoindol-2-yl)ethyl]-3-phenoxypyridinium bromide (1.10 g, 2.59 mmol) in 14 ml of conc. hydrobromic acid (48% strength in water) was stirred at 100° C. for 48 hours. On cooling, a solid precipitated. The solid was filtered off and the filtrate was concentrated on a rotary evaporator. The residue was stirred with methanol/tetrahydrofuran, and the solid was filtered off, washed with tetrahydrofuran and dried under high vacuum. This gave 593 mg (78% of theory) of the title compound.

¹H-NMR (400 MHz, DMSO-d6) δ [ppm]: −0.150 (0.75), 0.146 (0.60), 1.760 (0.99), 2.328 (0.99), 2.367 (1.04), 2.670 (0.97), 2.710 (0.91), 2.773 (0.63), 2.864 (0.52), 3.531 (7.25), 3.601 (0.99), 3.634 (0.60), 3.650 (1.14), 4.855 (10.05), 6.971 (0.78), 7.098 (0.97), 7.226 (0.80), 7.265 (13.82), 7.285 (16.00), 7.338 (3.84), 7.356 (8.82), 7.375 (5.30), 7.527 (11.00), 7.547 (14.84), 7.567 (7.68), 7.876 (1.08), 8.048 (6.30), 8.169 (4.92), 8.184 (5.20), 8.191 (7.94), 8.206 (8.17), 8.257 (6.68), 8.281 (3.99), 8.829 (5.54), 8.842 (5.33), 9.055 (9.73).

### Example 67A

### 1-[2-(1,3-Dioxo-1,3-dihydro-2H-isoindol-2-yl)ethyl]-4-(piperidin-1-yl)pyridinium bromide

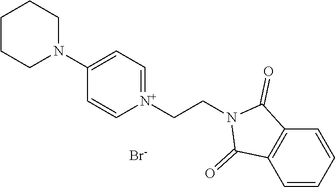 [SMILES: O=C1c2ccccc2C(=O)N1CC[n+]1ccc(N2CCCCC2)cc1 ; [Br-]]

4-(Piperidin-1-yl)pyridine (500 mg, 3.08 mmol) was initially charged in 4 ml of N,N-dimethylformamide, 2-(2-bromoethyl)-1H-isoindole-1,3(2H)-dione (783 mg, 3.08 mmol) was added and the mixture was stirred at 110° C. overnight. The precipitated solid was filtered off with suction, washed with methyl tert-butyl ether and dried under high vacuum. This gave 692 mg (100% pure, 54% of theory) of the title compound.

LC-MS (Method 2): Rₜ=0.96 min; MS (ESIpos): m/z=336 [M-Br-]⁺

### Example 68A

### 1-(2-Ammonioethyl)-4-(piperidin-1-yl)pyridinium dibromide

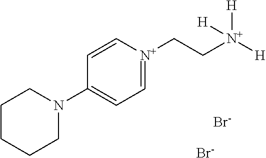 [SMILES: [NH3+]CC[n+]1ccc(N2CCCCC2)cc1 ; [Br-] ; [Br-]]

1-[2-(1,3-Dioxo-1,3-dihydro-2H-isoindol-2-yl)ethyl]-4-(piperidin-1-yl)pyridinium bromide (690 mg, 1.66 mmol) in 9 ml of aqueous hydrobromic acid (48% strength) was stirred at 100° C. for 3 hours. On cooling, a solid precipitated. The latter was filtered off and discarded. The filtrate was concentrated and the residue was stirred with tetrahydrofuran. The solid was filtered off, washed with tetrahydrofuran and dried under high vacuum. Once more, the solid was taken up in 9 ml of aqueous hydrobromic acid (48% strength) and the mixture was stirred at 110° C. for 1.5 days. The precipitated solid was then filtered off with suction and discarded. The filtrate was concentrated and the residue was stirred with tetrahydrofuran.

The solid was filtered off with suction, washed with tetrahydrofuran and dried under high vacuum. This gave 390 mg (64% of theory) of the title compound.

¹H-NMR (400 MHz, DMSO-d6) δ [ppm]: −0.008 (2.18), 0.008 (2.12), 1.596 (12.04), 1.605 (10.17), 1.689 (5.81), 1.701 (5.76), 3.333 (5.34), 3.346 (5.36), 3.479 (2.67), 3.542 (0.88), 3.695 (12.37), 3.708 (16.00), 3.722 (12.32), 4.367 (5.26), 4.382 (9.20), 4.396 (4.81), 7.280 (13.14), 7.300 (13.63), 8.011 (5.15), 8.207 (10.12), 8.225 (9.57).

### WORKING EXAMPLES

### Example 1

### 4-(Dimethylamino)-1-[2-({[3-(3,5-dimethyl-1,2-oxazol-4-yl)imidazo[1,2-a]pyridin-7-yl]carbonyl}amino)ethyl]pyridinium formate

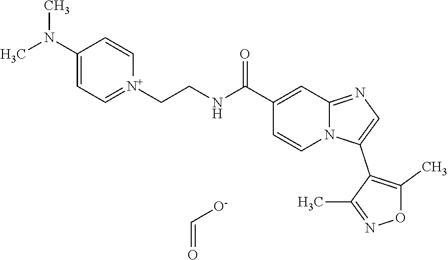

3-(3,5-Dimethyl-1,2-oxazol-4-yl)imidazo[1,2-a]pyridine-7-carboxylic acid (50.0 mg, 194 μmol) and 1-(2-aminoethyl)-4-(dimethylamino)pyridinium bromide hydrobromide (1:1:1) (63.6 mg, 194 μmol) were initially charged in 5 ml of dichloromethane, 1-(3-dimethylaminopropyl)-3-ethylcarbodiimide hydrochloride (55.9 mg, 292 μmol) and 4-dimethylaminopyridine (71.2 mg, 583 μmol) were added and the mixture was stirred at room temperature for 1.5 h. The reaction mixture was concentrated and the residue was purified directly by preparative HPLC (column: Chromatorex C18 10 μm, 250×30 mm, mobile phase A=water, B=acetonitrile; gradient: 0.0 min 5% B; 3 min 5% B; 20 min 50% B; 23 min 100% B; 26 min 5% B; flow rate: 50 ml/min; 0.1% formic acid). The product-containing fractions were combined, concentrated and lyophilized from acetonitrile/water overnight. 50 mg (56% of theory, 98% purity) of the title compound were obtained.

LC-MS (Method 2): Rₜ=0.63 min; MS (ESIpos): m/z=405 [M−HCO₂]⁺

¹H-NMR (400 MHz, DMSO-d6) δ [ppm]: 2.114 (10.41), 2.323 (10.60), 3.155 (16.00), 3.748 (1.56), 3.760 (1.60), 4.405 (1.64), 7.005 (2.22), 7.023 (2.22), 7.304 (0.94), 7.321 (0.91), 7.860 (2.03), 8.207 (2.56), 8.339 (1.60), 8.508 (1.17).

### Example 2

### 1-[2-({[3-(3,5-Dimethyl-1,2-oxazol-4-yl)imidazo[1,2-a]pyridin-7-yl]carbonyl}amino)ethyl]-4-(methylamino)pyridinium chloride hydrochloride (1:1:1)

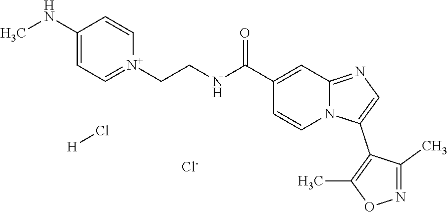

1-[2-({[3-(3,5-Dimethyl-1,2-oxazol-4-yl)imidazo[1,2-a]pyridin-7-yl]carbonyl}amino)ethyl]-4-(methylamino)pyridinium formate (500 mg, 1.15 mmol) was initially charged in 1.1 ml of 4 N aqueous hydrochloric acid and stirred for 5 minutes. Subsequently, the reaction mixture was concentrated. This operation was repeated four times. The residue was dissolved in 5 ml of water and lyophilized. 507 mg (96% of theory, 100% purity) of the title compound were obtained.

LC-MS (Method 2): Rₜ=0.60 min; MS (ESIpos): m/z=391 [M−HCl-Cl]⁺

¹H-NMR (400 MHz, DMSO-d6) δ [ppm]: 2.173 (15.69), 2.395 (16.00), 2.854 (5.59), 2.866 (5.59), 3.757 (0.80), 3.770 (1.79), 3.783 (1.85), 3.796 (0.88), 4.404 (1.51), 4.416 (2.17), 4.428 (1.38), 6.837 (1.00), 6.843 (1.21), 6.855 (1.07), 6.862 (1.24), 6.917 (0.98), 6.934 (0.97), 7.801 (1.01), 7.814 (0.74), 8.182 (1.32), 8.200 (1.30), 8.379 (1.33), 8.397 (1.32), 8.522 (3.44), 8.635 (1.76), 8.653 (1.67), 9.061 (0.52), 9.732 (0.62).

### Example 3

### 2-[({[3-(3,5-Dimethyl-1,2-oxazol-4-yl)imidazo[1,2-a]pyridin-7-yl]carbonyl}amino)methyl]-1-methylimidazo[1,2-a]pyridin-1-ium formate

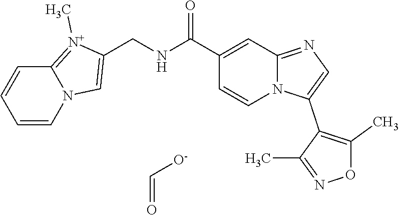

3-(3,5-Dimethyl-1,2-oxazol-4-yl)imidazo[1,2-a]pyridine-7-carboxylic acid (50.0 mg, 194 μmol) and (2-aminomethyl)-1-methylimidazo[1,2-a]pyridin-1-ium iodide hydrochloride (1:1:1) (63.3 mg, 194 μmol) were initially charged in 5 ml of dichloromethane, 1-(3-dimethylaminopropyl)-3-ethylcarbodiimide hydrochloride (55.9 mg, 292 μmol) and 4-dimethylaminopyridine (71.2 mg, 583 μmol) were added and the mixture was stirred at room temperature for one hour. The reaction mixture was concentrated and the residue was purified by preparative HPLC (column: Chromatorex C18 10 μm, 250×30 mm, mobile phase A=water, B=acetonitrile; gradient: 0.0 min 5% B; 3 min 5% B; 20 min 50% B; 23 min 100% B; 26 min 5% B; flow rate: 50 ml/min; 0.1% formic acid). The product-containing fractions were combined, concentrated and lyophilized from acetonitrile/water overnight. 55 mg (62% of theory, 99% purity) of the title compound were obtained.

LC-MS (Method 2): Rₜ=0.60 min; MS (ESIpos): m/z=401 [M−HCO₂]⁺

¹H-NMR (500 MHz, DMSO-d6) δ [ppm]: −0.007 (0.47), 1.563 (0.49), 2.116 (1.11), 2.124 (16.00), 2.146 (0.71), 2.325 (0.96), 2.333 (15.60), 3.495 (0.42), 3.898 (0.49), 4.073 (13.30), 4.856 (2.06), 4.866 (1.99), 7.442 (1.17), 7.445 (1.18), 7.457 (1.15), 7.460 (1.13), 7.522 (0.76), 7.535 (1.49), 7.549 (0.80), 7.884 (4.49), 8.007 (0.79), 8.025 (1.04), 8.040 (0.88), 8.206 (1.72), 8.225 (1.36), 8.262 (1.61), 8.277 (1.50), 8.352 (2.28), 8.427 (1.67), 8.546 (1.75), 8.899 (1.07), 8.912 (1.03), 10.079 (0.51), 10.089 (0.91), 10.100 (0.48).

### Example 4

### 1-[2-({[3-(3,5-Dimethyl-1,2-oxazol-4-yl)imidazo[1,2-a]pyridin-7-yl]carbonyl}amino)ethyl]-4-(methylamino)pyridinium formate

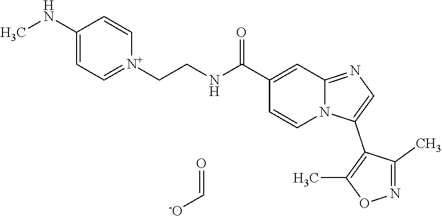

Preparative Method 1:

3-(3,5-Dimethyl-1,2-oxazol-4-yl)imidazo[1,2-a]pyridine-7-carboxylic acid (865 mg, 3.36 mmol) was initially charged in 10 ml of dichloromethane, 1-(2-ammonioethyl)-4-(methylamino)pyridinium dibromide (1.16 g, 3.70 mmol), 4-dimethylaminopyridine (1.23 g, 10.1 mmol) and 1-(3-dimethylaminopropyl)-3-ethylcarbodiimide hydrochloride (967 mg, 5.04 mmol) were added and the mixture was stirred at room temperature overnight. The reaction mixture was purified by silica gel chromatography (110 g Biotage NH-Snap Cartridge; Biotage-Isolera-One®; dichloromethane/methanol gradient 2% methanol—40% methanol; flow rate: 100 ml/min). The product fractions were combined and concentrated. The crude product was then dissolved in 10 ml water/acetonitrile, 3 ml of formic acid were added and the mixture was stirred for 20 min. The mixture was purified in several portions by preparative HPLC (column: Chromatorex C18 10 μm, 250×30 mm, mobile phase A=water, B=acetonitrile; gradient: 0.0 min 5% B; 3 min 5% B; 20 min 50% B; 23 min 100% B; 26 min 5% B; flow rate: 50 ml/min; 0.1% formic acid). This gave two product-containing fractions. The first product-containing fraction was concentrated and lyophilized. 167 mg (11% of theory, 100% purity) of the title compound were obtained.

LC-MS (Method 2): Rₜ=0.60 min; MS (ESIpos): m/z=391 [M−HCO₂]⁺

¹H-NMR (400 MHz, DMSO-d6) δ [ppm]: 2.114 (15.52), 2.323 (16.00), 2.854 (6.29), 3.714 (2.38), 3.725 (2.45), 4.332 (2.60), 6.835 (1.27), 6.854 (0.92), 6.886 (0.97), 7.290 (1.24), 7.304 (1.20), 7.863 (2.79), 8.119 (1.21), 8.177 (1.95), 8.217 (1.27), 8.228 (1.17), 8.312 (1.39), 8.329 (1.31), 8.541 (1.49).

The second product-containing fraction was concentrated, dissolved in 10 ml of acetonitrile/water, 3 ml of formic acid were added and the mixture was stirred for 1 h. The mixture was purified by preparative HPLC (column: Chromatorex C18 10 μm, 250×30 mm, mobile phase A=water, B=acetonitrile; gradient: 0.0 min 5% B; 3 min 5% B; 20 min 50% B; 23 min 100% B; 26 min 5% B; flow rate: 50 ml/min; 0.1% formic acid). The product-containing fractions were combined, concentrated and lyophilized. 360 mg (25% of theory, 100% purity) of the title compound were obtained.

LC-MS (Method 2): Rₜ=0.62 min; MS (ESIpos): m/z=391 [M−HCO₂]⁺

¹H-NMR (400 MHz, DMSO-d6) δ [ppm]: 2.116 (15.89), 2.325 (16.00), 2.857 (5.52), 2.869 (5.49), 3.700 (1.07), 3.714 (2.07), 3.726 (2.13), 3.740 (1.12), 4.316 (1.56), 4.330 (2.23), 4.342 (1.44), 6.842 (1.29), 6.856 (2.89), 6.873 (1.11), 7.274 (1.48), 7.277 (1.50), 7.295 (1.54), 7.866 (3.76), 8.105 (1.38), 8.123 (1.24), 8.164 (2.85), 8.222 (1.71), 8.240 (1.61), 8.310 (1.37), 8.327 (1.27), 8.449 (1.14), 8.922 (0.42), 9.085 (0.53).

Preparative Method 2:

Step 1: Charging the Ion Exchanger:

90 ml of Amberlite IRA 410 chloride form were charged into an empty cartridge. 500 ml of a 1 M aqueous sodium formate solution were passed over the ion exchanger, followed by 500 ml of water.

Step 2: Exchange Chloride/Formate:

1-[2-({[3-(3,5-Dimethyl-1,2-oxazol-4-yl)imidazo[1,2-a]pyridin-7-yl]carbonyl}amino)ethyl]-4-(methylamino)pyridinium chloride (1.00 g, 2.34 mmol) was dissolved in 3 ml of water and passed over the ion exchanger described in Step 1. The ion exchanger was washed with 250 ml of water and the combined filtrates were concentrated and dried under high vacuum. The residue was divided into three parts and purified by preparative HPLC (column: Chromatorex C18 10 m, 250×30 mm, mobile phase A=water, B=acetonitrile; gradient: 0.0 min 5% B; 3 min 5% B; 20 min 50% B; 23 min 100% B; 26 min 5% B; flow rate: 50 ml/min; 0.1% formic acid). The product fractions were combined, concentrated and lyophilized. This gave 787 mg (100% pure, 77% of theory) of the title compound.

LC-MS (Method 2): Rₜ=0.59 min; MS (ESIpos): m/z=391 [M−HCO₂]+

¹H-NMR (400 MHz, DMSO-d6) δ [ppm]: 2.115 (15.84), 2.324 (16.00), 2.856 (4.73), 2.868 (4.74), 3.698 (0.94), 3.712 (1.91), 3.724 (1.98), 3.738 (0.99), 4.311 (1.51), 4.325 (2.18), 4.337 (1.40), 6.842 (1.55), 6.853 (2.27), 6.860 (1.64), 7.272 (1.35), 7.290 (1.40), 7.864 (3.67), 8.100 (1.35), 8.118 (1.15), 8.161 (2.47), 8.220 (1.44), 8.238 (1.40), 8.306 (1.26), 8.323 (1.24), 8.440 (1.95).

### Example 5

### 1-[2-({[3-(3,5-Dimethyl-1,2-oxazol-4-yl)imidazo[1,2-a]pyridin-7-yl]carbonyl}amino)ethyl]-4-(methylamino)pyridinium chloride

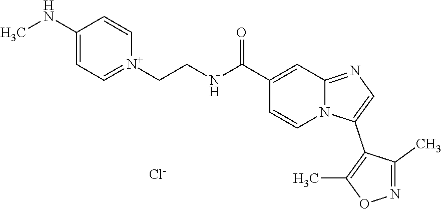

Sodium 3-(3,5-dimethyl-1,2-oxazol-4-yl)imidazo[1,2-a]pyridine-7-carboxylate (2.97 g, 10.64 mmol) and 1-(2-ammonioethyl)-4-(methylamino)pyridinium dichloride (2.38 g, 10.64 mmol) were initially charged in 30 ml of dichloromethane, 1-(3-dimethylaminopropyl)-3-ethylcarbodiimide hydrochloride (3.09 g, 16.1 mmol) and 4-dimethylaminopyridine (3.90 g, 31.9 mmol) were added and the mixture was stirred at room temperature overnight. The reaction mixture was applied to Isolute® and purified by column chromatography (375 g Biotage Snap Cartridge KP-NH®; Biotage-Isolera-One®; dichloromethane/methanol gradient 5% methanol—40% methanol; flow rate: 150 ml/min). The product-containing fractions were combined and concentrated by evaporation. The residue was dissolved in water and lyophilized. This gave 2.55 g (100% pure, 56% of theory) of the title compound.

LC-MS (Method 2): Rₜ=0.60 min; MS (ESIpos): m/z=391 [M-Cl-]⁺

¹H-NMR (400 MHz, DMSO-d6) δ [ppm]: 2.118 (15.72), 2.327 (16.00), 2.861 (11.94), 2.943 (0.44), 3.323 (1.28), 4.336 (1.43), 4.349 (2.05), 4.361 (1.30), 6.845 (1.22), 6.864 (2.11), 6.887 (1.07), 7.290 (1.49), 7.293 (1.39), 7.307 (1.50), 7.311 (1.44), 7.868 (5.44), 8.119 (1.20), 8.137 (1.24), 8.190 (2.69), 8.223 (2.08), 8.241 (1.97), 8.319 (1.35), 8.337 (1.24), 9.027 (0.44).

### Example 6

### 1-[2-({[3-(1,4-Dimethyl-1H-pyrazol-5-yl)imidazo[1,2-a]pyridin-7-yl]carbonyl}amino)ethyl]-4-(methylamino)pyridinium formate

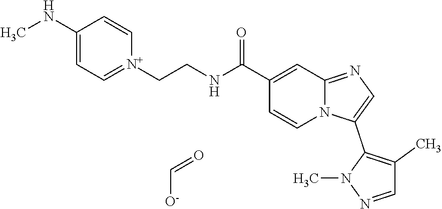

3-(1,4-Dimethyl-1H-pyrazol-5-yl)imidazo[1,2-a]pyridine-7-carboxylic acid (59.0 mg, 230 μmol) and 1-(2-ammonioethyl)-4-(methylamino)pyridinium dibromide (72.1 mg, 230 μmol) were initially charged in 5 ml of dichloromethane, 1-(3-dimethylaminopropyl)-3-ethylcarbodiimide hydrochloride (66.2 mg, 345 μmol) and 4-dimethylaminopyridine (84.4 mg, 691 μmol) were added and the mixture was stirred at room temperature for one hour. The reaction mixture was concentrated and the residue was purified directly by preparative HPLC (column: Chromatorex C18 10 μm, 250×30 mm, mobile phase A=water, B=acetonitrile; gradient: 0.0 min 5% B; 3 min 5% B; 20 min 50% B; 23 min 100% B; 26 min 5% B; flow rate: 50 ml/min; 0.1% formic acid). The product-containing fractions were combined and concentrated by evaporation. 41 mg (39% of theory, 96% purity) of the title compound were obtained.

LC-MS (Method 2): Rₜ=0.66 min; MS (ESIpos): m/z=390 [M−HCO₂]⁺

¹H-NMR (400 MHz, DMSO-d6) δ [ppm]: −0.008 (0.95), 0.008 (0.79), 1.069 (0.58), 1.872 (14.04), 2.103 (0.48), 2.144 (0.44), 2.860 (4.76), 3.637 (0.99), 3.652 (16.00), 3.717 (1.54), 3.729 (1.59), 3.901 (0.52), 4.315 (1.25), 4.329 (1.85), 4.341 (1.16), 6.839 (1.01), 6.856 (1.95), 6.874 (0.87), 6.880 (0.79), 7.316 (1.27), 7.334 (1.33), 7.508 (3.86), 7.980 (4.00), 8.046 (1.62), 8.064 (1.52), 8.112 (1.07), 8.129 (1.06), 8.205 (2.13), 8.312 (1.17), 8.330 (1.09), 8.557 (2.73), 9.144 (0.41).

### Example 7

### 1-[3-({[3-(3,5-Dimethyl-1,2-oxazol-4-yl)imidazo[1,2-a]pyridin-7-yl]carbonyl}amino)propyl]-4-(methylamino)pyridinium formate

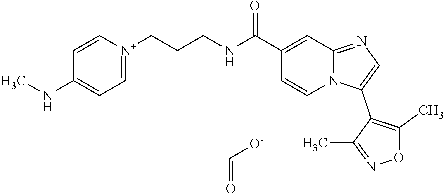

3-(3,5-Dimethyl-1,2-oxazol-4-yl)imidazo[1,2-a]pyridine-7-carboxylic acid (50.0 mg, 194 μmol) and 1-(3-aminopropyl)-4-(methylamino)pyridinium bromide hydrobromide (1:1:1) (63.6 mg, 194 μmol) were initially charged in 5 ml of dichloromethane, 1-(3-dimethylaminopropyl)-3-ethylcarbodiimide hydrochloride (55.9 mg, 292 μmol) and 4-dimethylaminopyridine (71.2 mg, 583 μmol) were added and the mixture was stirred at room temperature for 1.5 h. The reaction mixture was concentrated and the residue was purified directly by preparative HPLC (column: Chromatorex C18 10 μm, 250×30 mm, mobile phase A=water, B=acetonitrile; gradient: 0.0 min 5% B; 3 min 5% B; 20 min 50% B; 23 min 100% B; 26 min 5% B; flow rate: 50 ml/min; 0.1% formic acid). The product-containing fractions were combined, concentrated and dried under high vacuum. 41 mg (44% of theory, 95% purity) of the title compound were obtained.

LC-MS (Method 2): Rₜ=0.64 min; MS (ESIpos): m/z=405 [M−HCO₂]⁺

¹H-NMR (400 MHz, DMSO-d6) δ [ppm]: 2.035 (0.42), 2.052 (1.54), 2.068 (2.34), 2.085 (1.61), 2.112 (3.08), 2.123 (15.78), 2.152 (0.46), 2.320 (2.82), 2.333 (16.00), 2.849 (6.22), 2.861 (6.05), 3.157 (0.85), 3.300 (0.96), 3.316 (2.41), 3.330 (2.39), 3.346 (0.94), 4.207 (1.77), 4.224 (3.47), 4.241 (1.74), 6.850 (1.17), 6.868 (1.20), 6.912 (1.11), 6.929 (1.14), 7.361 (1.78), 7.381 (1.83), 7.697 (0.70), 7.863 (4.51), 8.163 (1.59), 8.166 (1.67), 8.181 (1.60), 8.184 (1.64), 8.230 (6.07), 8.248 (1.93), 8.364 (1.60), 8.382 (1.59), 8.432 (4.36), 8.943 (0.81), 9.088 (0.47).

### Example 8

### 1-[2-({[3-(1-Ethyl-1H-pyrazol-5-yl)imidazo[1,2-a]pyridin-7-yl]carbonyl}amino)ethyl]-4-(methylamino)pyridinium formate

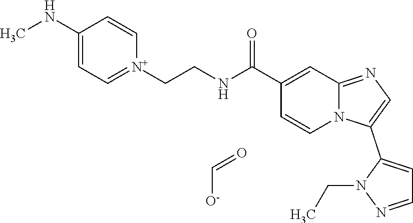

3-(1-Ethyl-1H-pyrazol-5-yl)imidazo[1,2-a]pyridine-7-carboxylic acid (39.0 mg, 152 μmol) and 1-(2-ammonioethyl)-4-(methylamino)pyridinium dibromide (47.6 mg, 152 μmol) were initially charged in 5 ml of dichloromethane, 1-(3-dimethylaminopropyl)-3-ethylcarbodiimide hydrochloride (43.8 mg, 228 μmol) and 4-dimethylaminopyridine (55.8 mg, 457 μmol) were added and the mixture was stirred at room temperature for one hour. The reaction mixture was concentrated and the residue was purified directly by preparative HPLC (column: Chromatorex C18 10 μm, 250×30 mm, mobile phase A=water, B=acetonitrile; gradient: 0.0 min 5% B; 3 min 5% B; 20 min 50% B; 23 min 100% B; 26 min 5% B; flow rate: 50 ml/min; 0.1% formic acid). The product-containing fractions were combined, concentrated and lyophilized from acetonitrile/water overnight. 15 mg (22% of theory, 96% purity) of the title compound were obtained.

LC-MS (Method 2): Rₜ=0.67 min; MS (ESIneg): m/z=388 [M−HCO₂-2H]-¹H-NMR (400 MHz, DMSO-d6) δ [ppm]: −0.149 (0.68), −0.008 (6.13), 0.008 (5.34), 0.146 (0.68), 1.272 (7.25), 1.290 (16.00), 1.308 (7.36), 2.150 (0.84), 2.328 (1.36), 2.367 (1.00), 2.670 (1.39), 2.710 (1.10), 2.860 (8.27), 2.869 (7.96), 3.714 (3.04), 3.727 (3.12), 4.063 (2.04), 4.081 (6.31), 4.099 (6.21), 4.117 (1.94), 4.323 (3.61), 6.692 (6.31), 6.697 (6.39), 6.838 (3.22), 6.847 (3.46), 6.857 (3.35), 7.319 (2.59), 7.337 (2.67), 7.716 (6.05), 7.720 (6.00), 7.793 (0.60), 8.011 (8.56), 8.100 (2.12), 8.115 (2.33), 8.184 (4.84), 8.280 (3.43), 8.298 (5.18), 8.316 (2.15), 8.559 (5.39), 8.792 (1.28), 9.022 (1.36).

### Example 9

### 1-[2-({[3-(2-Methoxypyridin-3-yl)imidazo[1,2-a]pyridin-7-yl]carbonyl}amino)ethyl]-4-(methylamino)pyridinium formate

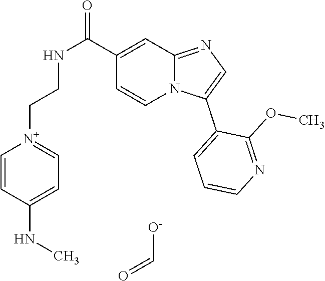

3-(2-Methoxypyridin-3-yl)imidazo[1,2-a]pyridine-7-carboxylic acid (47.7 mg, 92% pure, 163 μmol) and 1-(2-ammonioethyl)-4-(methylamino)pyridinium dibromide (51.1 mg, 163 μmol) were initially charged in 5.3 ml of dichloromethane, 1-(3-dimethylaminopropyl)-3-ethylcarbodiimide hydrochloride (46.9 mg, 245 μmol) and 4-dimethylaminopyridine (59.8 mg, 489 μmol) were added and the mixture was stirred at room temperature overnight. The reaction mixture was concentrated and the residue was purified directly by preparative HPLC (column: Chromatorex C18 10 μm, 125×40 mm, mobile phase A=water, B=acetonitrile; gradient: 0.0 min 5% B; 3 min 5% B; 20 min 50% B; 23 min 100% B; 26 min 5% B; flow rate: 50 ml/min; 0.1% formic acid). The product-containing fractions were combined, concentrated and dried under high vacuum. 49.5 mg (64% of theory, 95% purity) of the title compound were obtained.

LC-MS (Method 4): Rₜ=0.55 min; MS (ESIpos): m/z=403 [M−HCO₂]⁺

¹H-NMR (400 MHz, DMSO-d6) δ [ppm]: −0.008 (1.43), 0.008 (1.23), 2.154 (1.28), 2.857 (4.86), 2.869 (4.85), 2.942 (0.51), 3.715 (1.61), 3.728 (1.56), 3.901 (16.00), 4.315 (1.18), 4.330 (1.65), 4.342 (1.07), 6.837 (0.68), 6.843 (1.17), 6.858 (2.39), 6.874 (1.18), 6.880 (0.69), 7.181 (1.40), 7.193 (1.51), 7.199 (1.44), 7.212 (1.43), 7.263 (1.20), 7.267 (1.20), 7.281 (1.17), 7.285 (1.27), 7.865 (4.02), 7.908 (1.55), 7.913 (1.67), 7.927 (1.59), 7.931 (1.56), 8.110 (1.11), 8.128 (1.21), 8.137 (1.89), 8.156 (3.68), 8.307 (1.14), 8.325 (1.01), 8.340 (1.53), 8.345 (1.56), 8.353 (1.51), 8.358 (1.39), 8.488 (1.48), 8.903 (0.71), 8.915 (0.71), 9.044 (0.51), 9.058 (0.97), 9.071 (0.49).

### Example 10

### 3-[({[3-(3,5-Dimethyl-1,2-oxazol-4-yl)imidazo[1,2-a]pyridin-7-yl]carbonyl}amino)methyl]-1-methylpyridinium formate

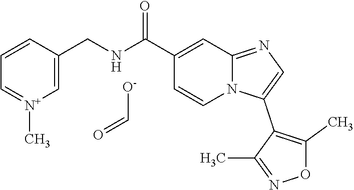

3-(3,5-Dimethyl-1,2-oxazol-4-yl)imidazo[1,2-a]pyridine-7-carboxylic acid (50.0 mg, 194 μmol) and 3-(aminomethyl)-1-methylpyridinium iodide hydrochloride (1:1:1) (55.7 mg, 194 μmol) were initially charged in 6 ml of dichloromethane, 1-(3-dimethylaminopropyl)-3-ethylcarbodiimide hydrochloride (55.9 mg, 292 μmol) and 4-dimethylaminopyridine (71.2 mg, 583 μmol) were added and the mixture was stirred at room temperature overnight. The reaction mixture was concentrated and the residue was purified directly by preparative HPLC (column: Chromatorex C18 10 μm, 125×40 mm, mobile phase A=water, B=acetonitrile; gradient: 0.0 min 5% B; 3 min 5% B; 20 min 50% B; 23 min 100% B; 26 min 5% B; flow rate: 50 ml/min; 0.1% formic acid). The product-containing fractions were combined, concentrated and re-purified by preparative HPLC (column: Chromatorex C18 10 μm, 250×30 mm, mobile phase A=water, B=acetonitrile; gradient: 0.0 min 5% B; 3 min 5% B; 20 min 50% B; 23 min 100% B; 26 min 5% B; flow rate: 50 ml/min; 0.1% formic acid). The product-containing fractions were combined, concentrated and dried under high vacuum. 78 mg (97% of theory, 98% purity) of the title compound were obtained.

LC-MS (Method 3): Rₜ=0.23 min; MS (ESIpos): m/z=362 [M−HCO₂]⁺

¹H-NMR (400 MHz, DMSO-d6) δ [ppm]: 2.124 (16.00), 2.334 (15.98), 3.431 (0.50), 4.365 (11.33), 4.688 (2.67), 4.702 (2.69), 5.755 (1.65), 7.388 (1.43), 7.392 (1.47), 7.406 (1.46), 7.410 (1.51), 7.891 (5.48), 8.094 (0.88), 8.109 (1.09), 8.114 (1.14), 8.129 (1.01), 8.274 (2.00), 8.292 (1.92), 8.318 (2.71), 8.391 (0.79), 8.537 (1.28), 8.557 (1.17), 8.890 (1.33), 8.905 (1.29), 9.022 (2.16), 9.603 (0.87).

### Example 11

### 1-[2-({[3-(3,5-Dimethyl-1,2-oxazol-4-yl)imidazo[1,2-a]pyridin-7-yl]carbonyl}amino)ethyl]-3-(methylamino)pyridinium formate

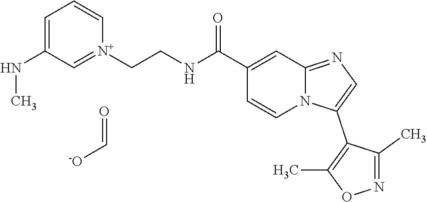

3-(3,5-Dimethyl-1,2-oxazol-4-yl)imidazo[1,2-a]pyridine-7-carboxylic acid (60.0 mg, 233 μmol) and 1-(2-ammonioethyl)-3-(methylamino)pyridinium dibromide (73.0 mg, 233 μmol) were initially charged in 3 ml of dichloromethane, 1-(3-dimethylaminopropyl)-3-ethylcarbodiimide hydrochloride (67.1 mg, 350 μmol) and 4-dimethylaminopyridine (85.5 mg, 700 μmol) were added and the mixture was stirred at room temperature for 72 hours. The reaction mixture was concentrated and the residue was purified directly by preparative HPLC (column: Chromatorex C18 10 μm, 250×30 mm, mobile phase A=water, B=acetonitrile; gradient: 0.0 min 5% B; 3 min 5% B; 20 min 50% B; 23 min 100% B; 26 min 5% B; flow rate: 50 ml/min; 0.1% formic acid). The product-containing fractions were combined, concentrated and dried under high vacuum. 43 mg (42% of theory, 99% purity) of the title compound were obtained.

LC-MS (Method 2): Rₜ=0.61 min; MS (ESIpos): m/z=391 [M−HCO₂]⁺

¹H-NMR (400 MHz, DMSO-d6) δ [ppm]: −0.149 (0.59), −0.008 (4.93), 0.008 (4.93), 0.146 (0.61), 2.115 (15.55), 2.147 (0.75), 2.323 (16.00), 2.366 (0.66), 2.388 (1.51), 2.670 (0.87), 2.710 (0.76), 2.732 (6.20), 2.745 (6.23), 3.845 (1.56), 3.858 (1.61), 4.611 (1.32), 4.624 (1.98), 4.637 (1.27), 7.180 (0.85), 7.190 (0.89), 7.243 (1.42), 7.247 (1.44), 7.261 (1.42), 7.265 (1.51), 7.580 (0.85), 7.602 (1.21), 7.607 (1.27), 7.681 (1.30), 7.695 (1.41), 7.702 (0.90), 7.717 (0.95), 7.871 (4.82), 8.127 (3.70), 8.139 (1.68), 8.154 (1.86), 8.233 (2.00), 8.251 (1.87), 8.378 (0.78), 8.911 (0.76).

### Example 12

### 3-Amino-1-[2-({[3-(3,5-dimethyl-1,2-oxazol-4-yl)imidazo[1,2-a]pyridin-7-yl]carbonyl}amino)ethyl]pyridinium formate

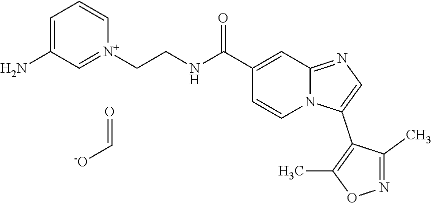

In the preparation of 1-[2-({[3-(3,5-dimethyl-1,2-oxazol-4-yl)imidazo[1,2-a]pyridin-7-yl]carbonyl}amino)ethyl]-3-(methylamino)pyridinium formate, 17 mg (17% of theory, 100% pure) of the title compound were obtained as a by-product.

LC-MS (Method 2): Rₜ=0.54 min; MS (ESIpos): m/z=377 [M−HCO₂]⁺

¹H-NMR (400 MHz, DMSO-d6) δ [ppm]: −0.149 (0.81), −0.008 (6.93), 0.008 (6.53), 0.146 (0.81), 2.073 (0.94), 2.114 (15.69), 2.322 (16.00), 2.366 (0.52), 2.670 (0.63), 2.710 (0.54), 2.941 (1.11), 3.805 (1.38), 3.817 (1.44), 4.592 (1.69), 6.634 (1.96), 7.260 (1.09), 7.278 (1.15), 7.547 (0.77), 7.572 (1.25), 7.642 (1.00), 7.656 (1.08), 7.678 (0.67), 7.868 (4.82), 8.102 (1.82), 8.116 (1.44), 8.140 (2.00), 8.223 (1.67), 8.241 (1.56), 8.554 (2.52).

### Example 13

### 4-Amino-1-[2-({[3-(3,5-dimethyl-1,2-oxazol-4-yl)imidazo[1,2-a]pyridin-7-yl]carbonyl}amino)ethyl]-2-methylpyridinium formate

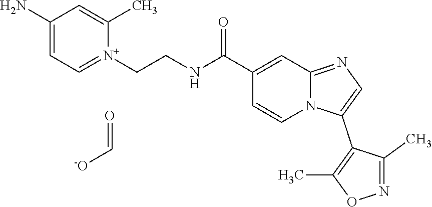

3-(3,5-Dimethyl-1,2-oxazol-4-yl)imidazo[1,2-a]pyridine-7-carboxylic acid (60.0 mg, 233 μmol) and 4-amino-1-(2-ammonioethyl)-2-methylpyridinium dibromide (73.0 mg, 233 μmol) were initially charged in 3 ml of dichloromethane, 1-(3-dimethylaminopropyl)-3-ethylcarbodiimide hydrochloride (67.1 mg, 350 μmol) and 4-dimethylaminopyridine (85.5 mg, 700 μmol) were added and the mixture was stirred at room temperature for 72 hours. The reaction mixture was concentrated and the residue was purified directly by preparative HPLC (column: Chromatorex C18 10 μm, 250×30 mm, mobile phase A=water, B=acetonitrile; gradient: 0.0 min 5% B; 3 min 5% B; 20 min 50% B; 23 min 100% B; 26 min 5% B; flow rate: 50 ml/min; 0.1% formic acid). The product-containing fractions were combined, concentrated and re-purified by preparative HPLC (column: Chromatorex C18 10 μm, 250×30 mm, mobile phase A=water, B=acetonitrile; gradient: 0.0 min 5% B; 3 min 5% B; 20 min 50% B; 23 min 100% B; 26 min 5% B; flow rate: 50 ml/min; 0.1% formic acid). The product-containing fractions were combined, concentrated and dried under high vacuum. 36 mg (34% of theory, 96% purity) of the title compound were obtained.

LC-MS (Method 2): Rₜ=0.61 min; MS (ESIpos): m/z=391 [M−HCO₂]⁺

¹H-NMR (400 MHz, DMSO-d6) δ [ppm]: 1.561 (0.80), 1.573 (0.80), 2.118 (15.68), 2.147 (0.98), 2.326 (16.00), 2.352 (0.47), 2.630 (13.63), 2.670 (0.59), 2.709 (0.44), 3.462 (1.08), 3.778 (2.45), 3.792 (2.52), 3.806 (1.26), 3.895 (1.03), 4.558 (1.57), 4.572 (2.78), 4.586 (1.53), 6.331 (2.50), 7.274 (1.43), 7.292 (1.49), 7.511 (0.86), 7.516 (0.86), 7.533 (1.74), 7.538 (1.83), 7.574 (2.11), 7.596 (1.02), 7.874 (3.58), 7.981 (1.04), 8.150 (2.89), 8.235 (1.56), 8.253 (1.48), 8.400 (2.24), 8.408 (2.28).

### Example 14

### 1-[3-({[3-(3,5-Dimethyl-1,2-oxazol-4-yl)imidazo[1,2-a]pyridin-7-yl]carbonyl}amino)propyl]-3-(methylamino)pyridinium formate

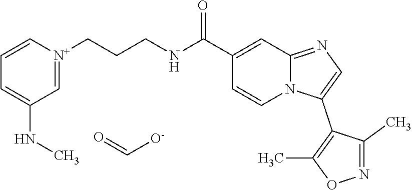

3-(3,5-Dimethyl-1,2-oxazol-4-yl)imidazo[1,2-a]pyridine-7-carboxylic acid (60.0 mg, 233 μmol) and 1-(3-ammoniopropyl)-3-(methylamino)pyridinium dibromide (76.3 mg, 233 μmol) were initially charged in 7.5 ml of dichloromethane, 1-(3-dimethylaminopropyl)-3-ethylcarbodiimide hydrochloride (67.1 mg, 350 μmol) and 4-dimethylaminopyridine (85.5 mg, 700 μmol) were added and the mixture was stirred at room temperature overnight. The reaction mixture was concentrated and the residue was purified directly by preparative HPLC (column: Chromatorex C18 10 μm, 250×30 mm, mobile phase A=water, B=acetonitrile; gradient: 0.0 min 5% B; 3 min 5% B; 20 min 50% B; 23 min 100% B; 26 min 5% B; flow rate: 50 ml/min; 0.1% formic acid). The product-containing fractions were combined, concentrated and dried under high vacuum. 42 mg (40% of theory, 100% purity) of the title compound were obtained.

LC-MS (Method 2): Rₜ=0.67 min; MS (ESIneg): m/z=403 [M−2H-HCO₂]⁻

¹H-NMR (400 MHz, DMSO-d6) δ [ppm]: −0.008 (1.55), 0.008 (1.43), 2.124 (15.80), 2.146 (1.04), 2.194 (1.14), 2.210 (1.69), 2.227 (1.16), 2.323 (1.25), 2.333 (16.00), 2.524 (0.90), 2.780 (5.84), 2.792 (5.89), 3.357 (5.15), 3.371 (5.15), 4.526 (1.27), 4.543 (2.52), 4.561 (1.23), 7.362 (1.52), 7.383 (1.44), 7.568 (0.90), 7.573 (0.93), 7.590 (1.17), 7.595 (1.21), 7.714 (1.08), 7.728 (1.17), 7.735 (0.85), 7.750 (0.85), 7.867 (4.82), 8.213 (1.52), 8.227 (1.63), 8.239 (5.30), 8.257 (2.60), 8.525 (2.73), 8.948 (0.47).

### Example 15

### 2-[({[3-(3,5-Dimethyl-1,2-oxazol-4-yl)imidazo[1,2-a]pyridin-7-yl]carbonyl}amino)methyl]-1,4-dimethylpyridinium formate

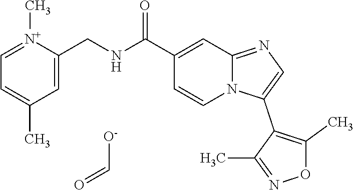

3-(3,5-Dimethyl-1,2-oxazol-4-yl)imidazo[1,2-a]pyridine-7-carboxylic acid (50.0 mg, 194 μmol) and 2-(aminomethyl)-1,4-dimethylpyridinium iodide hydrochloride (1:1:1) (58.4 mg, 194 μmol) were initially charged in 15 ml of dichloromethane, 1-(3-dimethylaminopropyl)-3-ethylcarbodiimide hydrochloride (55.9 mg, 292 μmol) and 4-dimethylaminopyridine (71.2 mg, 583 μmol) were added and the mixture was stirred at room temperature overnight. The reaction mixture was concentrated and the residue was purified by preparative HPLC (column: Chromatorex C18 10 μm, 125×40 mm, mobile phase A=water, B=acetonitrile; gradient: 0.0 min 5% B; 3 min 5% B; 20 min 50% B; 23 min 100% B; 26 min 5% B; flow rate: 50 ml/min; 0.1% formic acid). The product-containing fractions were combined, concentrated and dried under high vacuum. This gave 35 mg (98% pure, 42% of theory) of the title compound.

LC-MS (Method 2): Rₜ=0.63 min; MS (ESIpos): m/z=376 [M−HCO₂]⁺

¹H-NMR (400 MHz, DMSO-d6) δ [ppm]: −0.008 (1.03), 0.008 (0.81), 2.129 (16.00), 2.329 (1.46), 2.338 (15.97), 2.464 (0.46), 2.579 (10.08), 4.322 (10.68), 4.360 (0.46), 4.890 (2.68), 4.903 (2.71), 5.754 (1.84), 7.425 (1.44), 7.429 (1.49), 7.443 (1.46), 7.447 (1.49), 7.848 (1.19), 7.864 (1.14), 7.905 (5.07), 7.919 (2.28), 8.280 (1.99), 8.298 (1.88), 8.396 (2.62), 8.502 (1.83), 8.850 (2.03), 8.866 (1.97), 9.909 (0.75).

### Example 16

### 1-[2-({[3-(3,5-Dimethyl-1,2-oxazol-4-yl)imidazo[1,2-a]pyridin-7-yl]carbonyl}amino)ethyl]-3-methyl-4-(methylamino)pyridinium formate

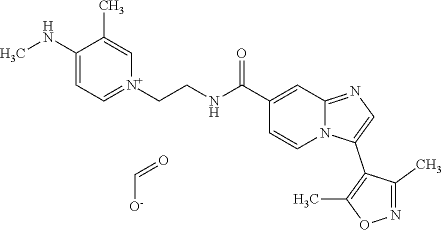

Sodium 3-(3,5-dimethyl-1,2-oxazol-4-yl)imidazo[1,2-a]pyridine-7-carboxylate (50.0 mg, 179 μmol) and 1-(2-aminoethyl)-3-methyl-4-(methylamino)pyridinium chloride hydrochloride (1:1:1) (46.9 mg, 197 μmol) were initially charged in 2 ml of dichloromethane, 1-(3-dimethylaminopropyl)-3-ethylcarbodiimide hydrochloride (51.5 mg, 269 μmol) and 4-dimethylaminopyridine (65.6 mg, 537 μmol) were added and the mixture was stirred at room temperature overnight. The reaction mixture was concentrated, the residue was taken up in methanol, 0.5 ml of formic acid was added and evaporated over a period of 15 minutes on a rotary evaporator at 50° C. Subsequently, the mixture was purified by preparative HPLC (column: Chromatorex C18 10 μm, 125×40 mm, mobile phase A=water, B=acetonitrile; gradient: 0.0 min 10% B; 5 min 10% B; 19 min 50% B; 20 min 95% B; 26 min 10% B; flow rate: 100 ml/min; 0.1% formic acid). The product-containing fractions were combined and concentrated and the residue was dissolved in water/acetonitrile and lyophilized. This gave 43.3 mg (100% pure, 54% of theory) of the title compound.

LC-MS (Method 2): Rₜ=0.65 min; MS (ESIpos): m/z=405 [M−HCO₂]⁺

¹H-NMR (400 MHz, DMSO-d6) δ [ppm]: 2.080 (10.98), 2.115 (16.00), 2.146 (0.61), 2.324 (15.78), 2.908 (5.10), 2.915 (4.84), 3.447 (1.02), 3.733 (1.81), 3.744 (1.83), 4.349 (2.08), 6.825 (1.53), 6.843 (1.54), 7.285 (1.34), 7.303 (1.37), 7.861 (2.13), 8.114 (0.63), 8.169 (2.44), 8.215 (1.31), 8.238 (2.79), 8.295 (1.11), 8.312 (1.08), 8.551 (0.77).

### Example 17

### 1-[2-({[3-(3,5-Dimethyl-1,2-oxazol-4-yl)imidazo[1,2-a]pyridin-7-yl]carbonyl}amino)ethyl]-2-methyl-4-(methylamino)pyridinium formate

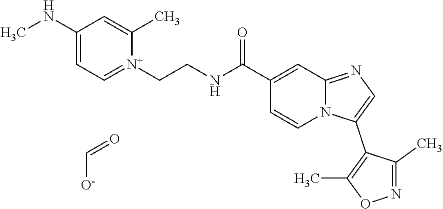

Sodium 3-(3,5-dimethyl-1,2-oxazol-4-yl)imidazo[1,2-a]pyridine-7-carboxylate (50.0 mg, 179 μmol) and 1-(2-aminoethyl)-2-methyl-4-(methylamino)pyridinium chloride hydrochloride (1:1:1) (46.9 mg, 197 μmol) were initially charged in 2 ml of dichloromethane, 1-(3-dimethylaminopropyl)-3-ethylcarbodiimide hydrochloride (51.5 mg, 269 μmol) and 4-dimethylaminopyridine (65.6 mg, 537 μmol) were added and the mixture was stirred at room temperature overnight. The reaction mixture was concentrated, the residue was taken up in methanol, 0.5 ml of formic acid was added and evaporated over a period of 15 minutes on a rotary evaporator at 50° C. Subsequently, the mixture was purified by preparative HPLC (column: Chromatorex C18 10 μm, 125×40 mm, mobile phase A=water, B=acetonitrile; gradient: 0.0 min 10% B; 5 min 10% B; 19 min 50% B; 20 min 95% B; 26 min 10% B; flow rate: 100 ml/min; 0.1% formic acid). The product-containing fractions were combined and concentrated and the residue was dissolved in water/acetonitrile and lyophilized. This gave 36 mg (100% pure, 45% of theory) of the title compound.

LC-MS (Method 2): Rₜ=0.69 min; MS (ESIpos): m/z=405 [M−HCO₂]⁺

¹H-NMR (400 MHz, DMSO-d6) δ [ppm]: −0.008 (1.39), 0.008 (0.90), 2.074 (0.54), 2.119 (16.00), 2.146 (0.83), 2.327 (15.93), 2.606 (6.17), 2.828 (2.07), 2.849 (3.06), 2.859 (2.53), 3.434 (1.28), 3.692 (2.02), 3.705 (2.00), 4.331 (1.79), 4.344 (1.55), 6.747 (1.60), 6.800 (1.09), 7.291 (1.07), 7.307 (1.06), 7.868 (1.71), 8.024 (0.52), 8.041 (0.59), 8.173 (1.63), 8.229 (1.42), 8.245 (1.28), 8.534 (0.43).

### Example 18

### 1-[2-({[3-(3,5-Dimethyl-1,2-oxazol-4-yl)imidazo[1,2-a]pyridin-7-yl]carbonyl}amino)ethyl]-4-(ethylamino)pyridinium formate

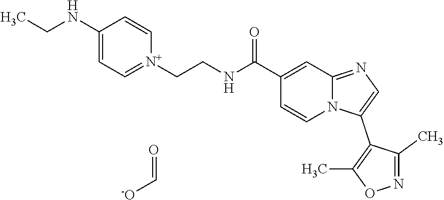

Sodium 3-(3,5-dimethyl-1,2-oxazol-4-yl)imidazo[1,2-a]pyridine-7-carboxylate (100 mg, 358 μmol) and 1-(2-ammonioethyl)-4-(ethylamino)pyridinium dichloride (93.8 mg, 394 μmol) were initially charged in 2 ml of dichloromethane, 1-(3-dimethylaminopropyl)-3-ethylcarbodiimide hydrochloride (103 mg, 537 μmol) and 4-dimethylaminopyridine (131 mg, 1.07 mmol) were added and the mixture was stirred at room temperature over the weekend. The reaction mixture was concentrated and the residue was purified by preparative HPLC (column: Chromatorex C18 10 μm, 250×30 mm, mobile phase A=water, B=acetonitrile; gradient: 0.0 min 5% B; 3 min 5% B; 20 min 50% B; 23 min 100% B; 26 min 5% B; flow rate: 50 ml/min; 0.1% formic acid). The product-containing fractions were combined and concentrated and the residue was dissolved in water/acetonitrile and lyophilized. This gave 112 mg (100% pure, 69% of theory) of the title compound.

LC-MS (Method 2): Rₜ=0.71 min; MS (ESIpos): m/z=405 [M−HCO₂]⁺

¹H-NMR (400 MHz, DMSO-d6) δ [ppm]: −0.008 (1.05), 0.008 (1.00), 1.136 (3.32), 1.154 (7.09), 1.172 (3.42), 2.115 (15.93), 2.324 (16.00), 3.228 (0.56), 3.246 (1.48), 3.263 (1.75), 3.277 (1.54), 3.295 (0.77), 3.712 (1.57), 3.723 (1.61), 4.325 (1.88), 6.857 (1.83), 6.863 (1.80), 6.875 (1.81), 7.282 (1.31), 7.300 (1.35), 7.862 (4.54), 8.111 (0.97), 8.129 (1.04), 8.171 (2.38), 8.216 (1.74), 8.234 (1.63), 8.281 (1.01), 8.297 (0.96), 8.549 (2.43).

### Example 19

### 1-[2-({[3-(3,5-Dimethyl-1,2-oxazol-4-yl)imidazo[1,2-a]pyridin-7-yl]carbonyl}amino)ethyl]-3-ethylpyridinium formate

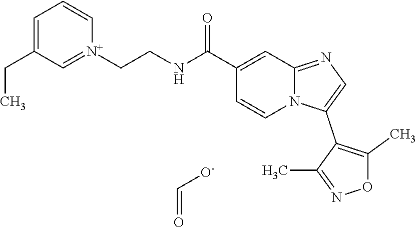

3-(3,5-Dimethyl-1,2-oxazol-4-yl)imidazo[1,2-a]pyridine-7-carboxylic acid (50.0 mg, 194 μmol) and 1-(2-aminoethyl)-3-ethylpyridinium bromide hydrobromide (1:1:1) (60.7 mg, 194 μmol) were initially charged in 5 ml of dichloromethane, 1-(3-dimethylaminopropyl)-3-ethylcarbodiimide hydrochloride (55.9 mg, 292 μmol) and 4-dimethylaminopyridine (71.2 mg, 583 μmol) were added and the mixture was stirred at room temperature for three hours. The reaction mixture was concentrated and the residue was purified by preparative HPLC (column: Chromatorex C18 10 μm, 250×30 mm, mobile phase A=water, B=acetonitrile; gradient: 0.0 min 5% B; 3 min 5% B; 20 min 50% B; 23 min 100% B; 26 min 5% B; flow rate: 50 ml/min; 0.1% formic acid). The product-containing fractions were combined and concentrated and the residue was dissolved in water/acetonitrile and lyophilized. This gave 67 mg (99% pure, 79% of theory) of the title compound.

LC-MS (Method 2): Rₜ=0.66 min; MS (ESIpos): m/z=390 [M−HCO₂]⁺

¹H-NMR (400 MHz, DMSO-d6) δ [ppm]: 1.125 (3.30), 1.144 (6.98), 1.162 (3.39), 2.108 (15.98), 2.317 (16.00), 2.728 (0.97), 2.747 (2.86), 2.766 (2.79), 2.785 (0.89), 3.434 (0.61), 3.899 (0.76), 3.913 (1.75), 3.926 (1.78), 3.939 (0.79), 4.817 (1.94), 7.271 (1.24), 7.289 (1.28), 7.853 (3.00), 8.019 (0.97), 8.035 (1.22), 8.039 (1.24), 8.055 (1.07), 8.188 (4.11), 8.206 (1.49), 8.457 (1.37), 8.478 (1.25), 8.585 (1.06), 9.008 (1.04), 9.021 (1.01), 9.153 (1.48).

### Example 20

### 2-[({[3-(3,5-Dimethyl-1,2-oxazol-4-yl)imidazo[1,2-a]pyridin-7-yl]carbonyl}amino)methyl]-1-methylpyridinium formate

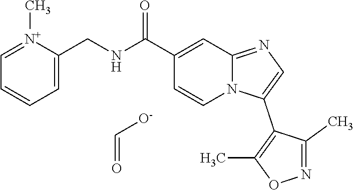

Sodium 3-(3,5-dimethyl-1,2-oxazol-4-yl)imidazo[1,2-a]pyridine-7-carboxylate (50.0 mg, 179 μmol) and 2-(aminomethyl)-1-methylpyridinium iodide hydrochloride (1:1:1) (51.3 mg, 179 μmol) were initially charged in 5 ml of dichloromethane, 1-(3-dimethylaminopropyl)-3-ethylcarbodiimide hydrochloride (51.5 mg, 269 μmol) and 4-dimethylaminopyridine (65.6 mg, 537 μmol) were added and the mixture was stirred at room temperature for 48 hours. The reaction mixture was concentrated and the residue was purified by preparative HPLC (column: Chromatorex C18 10 μm, 250×30 mm, mobile phase A=water, B=acetonitrile; gradient: 0.0 min 5% B; 3 min 5% B; 20 min 50% B; 23 min 100% B; 26 min 5% B; flow rate: 50 ml/min; 0.1% formic acid). The product-containing fractions were combined, concentrated and dried under high vacuum. This gave 40 mg (100% pure, 55% of theory) of the title compound.

LC-MS (Method 3): Rₜ=0.26 min; MS (ESIpos): m/z=362 [M−HCO₂]⁺

¹H-NMR (400 MHz, DMSO-d6) δ [ppm]: −0.008 (0.60), 0.008 (0.54), 2.075 (0.57), 2.130 (16.00), 2.285 (0.57), 2.339 (15.95), 2.942 (0.95), 3.408 (1.20), 4.411 (11.92), 4.951 (2.75), 4.964 (2.70), 7.429 (1.36), 7.432 (1.33), 7.447 (1.37), 7.450 (1.34), 7.906 (4.54), 8.008 (0.75), 8.025 (1.40), 8.042 (0.79), 8.086 (1.51), 8.106 (1.61), 8.283 (1.85), 8.301 (1.75), 8.394 (2.71), 8.495 (1.19), 8.516 (0.98), 8.535 (1.54), 8.554 (0.72), 9.023 (1.57), 9.038 (1.51), 10.094 (0.45).

### Example 21

### 1-[2-({[3-(3,5-Dimethyl-1,2-oxazol-4-yl)imidazo[1,2-a]pyridin-7-yl]carbonyl}amino)ethyl]-4-(trifluoromethyl)pyridinium formate

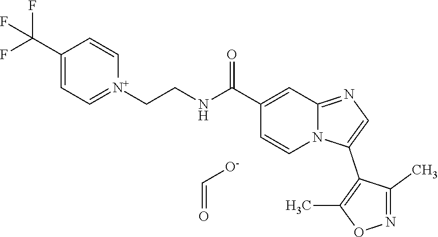

Sodium 3-(3,5-dimethyl-1,2-oxazol-4-yl)imidazo[1,2-a]pyridine-7-carboxylate (100 mg, 358 μmol) and 1-(2-ammonioethyl)-4-(trifluoromethyl)pyridinium dibromide (107 mg, 394 μmol) were initially charged in 2 ml of dichloromethane, 1-(3-dimethylaminopropyl)-3-ethylcarbodiimide hydrochloride (103 mg, 537 μmol) and 4-dimethylaminopyridine (131 mg, 1.07 mmol) were added and the mixture was stirred at room temperature overnight. The reaction mixture was concentrated and the residue was purified by preparative HPLC (column: Chromatorex C18 10 μm, 250×30 mm, mobile phase A=water, B=acetonitrile; gradient: 0.0 min 5% B; 3 min 5% B; 20 min 50% B; 23 min 100% B; 26 min 5% B; flow rate: 50 ml/min; 0.1% formic acid). The product-containing fractions were combined, concentrated and dried under high vacuum. The residue was re-purified by preparative HPLC (column: Chromatorex C18 10 μm, 250×30 mm, mobile phase A=water, B=acetonitrile; gradient: 0.0 min 5% B; 3 min 5% B; 20 min 50% B; 23 min 100% B; 26 min 5% B; flow rate: 50 ml/min; 0.1% formic acid). The product-containing fractions were combined, concentrated and dried under high vacuum. This gave 50 mg (91% pure, 27% of theory) of the title compound.

LC-MS (Method 2): Rₜ=0.72 min; MS (ESIpos): m/z=430 [M−HCO₂]⁺

¹H-NMR (400 MHz, DMSO-d6) δ [ppm]: 2.110 (15.91), 2.122 (2.59), 2.318 (16.00), 2.330 (2.79), 2.891 (0.43), 3.469 (1.48), 3.485 (1.48), 3.935 (1.85), 3.947 (1.88), 4.938 (2.18), 7.232 (1.47), 7.249 (1.51), 7.852 (0.60), 7.865 (4.73), 8.147 (2.95), 8.219 (2.13), 8.237 (2.06), 8.460 (3.96), 8.688 (2.87), 8.703 (2.94), 9.367 (0.52), 9.500 (2.58), 9.515 (2.48).

### Example 22

### 2-[({[3-(3,5-Dimethyl-1,2-oxazol-4-yl)imidazo[1,2-a]pyridin-7-yl]carbonyl}amino)methyl]-1,5-dimethylpyridinium formate

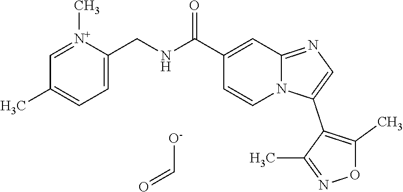

3-(3,5-Dimethyl-1,2-oxazol-4-yl)imidazo[1,2-a]pyridine-7-carboxylic acid (50.0 mg, 194 μmol) and 2-(aminomethyl)-1,5-dimethylpyridinium iodide hydrochloride (1:1:1) (58.4 mg, 194 μmol) were initially charged in 6 ml of dichloromethane, 1-(3-dimethylaminopropyl)-3-ethylcarbodiimide hydrochloride (55.9 mg, 292 μmol) and 4-dimethylaminopyridine (71.2 mg, 583 μmol) were added and the mixture was stirred at room temperature overnight. The reaction mixture was concentrated and the residue was purified by preparative HPLC (column: Chromatorex C18 10 μm, 125×40 mm, mobile phase A=water, B=acetonitrile; gradient: 0.0 min 5% B; 3 min 5% B; 20 min 50% B; 23 min 100% B; 26 min 5% B; flow rate: 50 ml/min; 0.1% formic acid). The product-containing fractions were combined, concentrated and dried under high vacuum. The residue was re-purified by preparative HPLC (column: Chromatorex C18 10 μm, 125×40 mm, mobile phase A=water, B=acetonitrile; gradient: 0.0 min 5% B; 3 min 5% B; 20 min 50% B; 23 min 100% B; 26 min 5% B; flow rate: 50 ml/min; 0.1% formic acid). The product-containing fractions were combined, concentrated and dried under high vacuum. This gave 47 mg (91% pure, 52% of theory) of the title compound.

LC-MS (Method 2): Rₜ=0.61 min; MS (ESIpos): m/z=376 [M−HCO₂]⁺

¹H-NMR (400 MHz, DMSO-d6) δ [ppm]: −0.008 (0.42), 0.008 (0.40), 1.398 (0.59), 2.075 (1.24), 2.115 (0.69), 2.122 (1.83), 2.130 (15.87), 2.154 (0.61), 2.323 (0.73), 2.330 (1.75), 2.339 (16.00), 2.431 (0.42), 2.469 (10.38), 4.360 (11.57), 4.899 (2.66), 4.912 (2.67), 7.399 (1.33), 7.403 (1.39), 7.417 (1.38), 7.421 (1.45), 7.908 (4.87), 7.976 (1.90), 7.997 (2.08), 8.288 (2.11), 8.306 (2.01), 8.345 (0.78), 8.364 (3.69), 8.388 (1.21), 8.950 (2.35), 9.758 (0.62).

### Example 23

### 1-[2-({[3-(3,5-Dimethyl-1,2-oxazol-4-yl)imidazo[1,2-a]pyridin-7-yl]carbonyl}amino)ethyl]-3-methylpyridinium formate

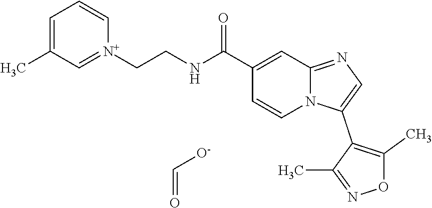

3-(3,5-Dimethyl-1,2-oxazol-4-yl)imidazo[1,2-a]pyridine-7-carboxylic acid (50.0 mg, 194 μmol) and 1-(2-aminoethyl)-3-methylpyridinium bromide hydrobromide (1:1:1) (57.9 mg, 194 μmol) were initially charged in 5 ml of dichloromethane, 1-(3-dimethylaminopropyl)-3-ethylcarbodiimide hydrochloride (55.9 mg, 292 μmol) and 4-dimethylaminopyridine (71.2 mg, 583 μmol) were added and the mixture was stirred at room temperature for three hours. The reaction mixture was concentrated and the residue was purified by preparative HPLC (column: Chromatorex C18 10 μm, 250×30 mm, mobile phase A=water, B=acetonitrile; gradient: 0.0 min 5% B; 3 min 5% B; 20 min 50% B; 23 min 100% B; 26 min 5% B; flow rate: 50 ml/min; 0.1% formic acid). The product-containing fractions were combined and concentrated and the residue was dissolved in water/acetonitrile and lyophilized. This gave 53 mg (100% pure, 65% of theory) of the title compound.

LC-MS (Method 2): Rₜ=0.58 min; MS (ESIpos): m/z=376 [M−HCO₂]⁺

¹H-NMR (400 MHz, DMSO-d6) δ [ppm]: 2.140 (16.00), 2.354 (15.94), 2.478 (11.38), 3.892 (0.86), 3.906 (1.85), 3.919 (1.88), 3.933 (0.86), 4.749 (1.52), 4.762 (2.21), 4.775 (1.36), 7.416 (1.00), 7.433 (1.01), 8.017 (1.13), 8.033 (1.30), 8.037 (1.38), 8.052 (1.24), 8.151 (1.57), 8.226 (2.29), 8.414 (1.39), 8.432 (1.34), 8.458 (1.39), 8.478 (1.26), 8.907 (1.48), 8.922 (1.41), 9.068 (2.40), 9.114 (0.81).

### Example 24

### 1-[3-({[3-(3,5-Dimethyl-1,2-oxazol-4-yl)imidazo[1,2-a]pyridin-7-yl]carbonyl}amino)propyl]-2,4-dimethyl-1H-pyrazol-2-ium formate

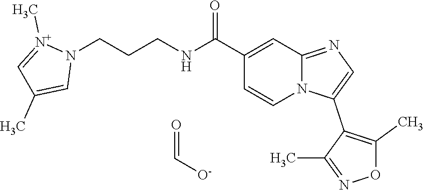

Sodium 3-(3,5-dimethyl-1,2-oxazol-4-yl)imidazo[1,2-a]pyridine-7-carboxylate (50.0 mg, 179 μmol) and 1-(3-aminopropyl)-2,4-dimethyl-1H-pyrazol-2-ium formate hydrochloride (1:1:1) (62.6 mg, 197 μmol) were initially charged in 2 ml of dichloromethane, 1-(3-dimethylaminopropyl)-3-ethylcarbodiimide hydrochloride (51.5 mg, 269 μmol) and 4-dimethylaminopyridine (65.6 mg, 537 μmol) were added and the mixture was stirred at room temperature overnight. The reaction mixture was concentrated and the residue was dissolved in formic acid and purified by preparative HPLC (column: Chromatorex C18 10 μm, 125×40 mm, mobile phase A=water, B=acetonitrile; gradient: 0.0 min 5% B; 3 min 5% B; 20 min 50% B; 23 min 100% B; 26 min 5% B; flow rate: 50 ml/min; 0.1% formic acid). The product-containing fractions were combined, concentrated and dried under high vacuum. This gave 62 mg (96% pure, 76% of theory) of the title compound.

LC-MS (Method 2): Rₜ=0.62 min; MS (ESIpos): m/z=393 [M−HCO₂]⁺

¹H-NMR (400 MHz, DMSO-d6) δ [ppm]: 2.087 (11.28), 2.123 (16.00), 2.133 (2.26), 2.150 (2.21), 2.168 (0.51), 2.332 (15.49), 3.356 (1.34), 3.371 (2.57), 3.386 (2.57), 3.401 (1.36), 3.707 (0.43), 4.087 (14.14), 4.472 (1.46), 4.490 (2.84), 4.507 (1.43), 5.755 (3.87), 7.352 (1.26), 7.370 (1.31), 7.870 (4.41), 8.226 (2.59), 8.247 (1.81), 8.265 (1.72), 8.298 (2.94), 8.380 (1.55), 8.418 (2.28), 8.905 (0.54).

### Example 25

### 1-[2-({[3-(1-Isopropyl-1H-pyrazol-5-yl)imidazo[1,2-a]pyridin-7-yl]carbonyl}amino)ethyl]-4-(methylamino)pyridinium formate

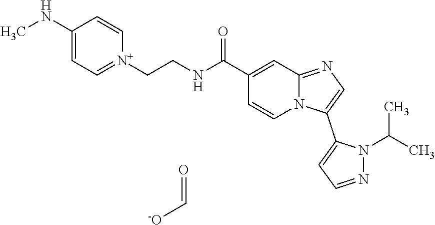

Lithium 3-(1-isopropyl-1H-pyrazol-5-yl)imidazo[1,2-a]pyridine-7-carboxylate (90.0 mg, 326 μmol) and 1-(2-ammonioethyl)-4-(methylamino)pyridinium dibromide (112 mg, 358 μmol) were initially charged in 5 ml of dichloromethane, 1-(3-dimethylaminopropyl)-3-ethylcarbodiimide hydrochloride (93.7 mg, 489 μmol) and 4-dimethylaminopyridine (119 mg, 977 μmol) were added and the mixture was stirred at room temperature for 48 hours. More 1-(2-ammonioethyl)-4-(methylamino)pyridinium dibromide (50.0 mg, 160 μmol), 1-(3-dimethylaminopropyl)-3-ethylcarbodiimide hydrochloride (50.0 mg, 260 μmol) and 4-dimethylaminopyridine (50.0 mg, 409 μmol) were added and the mixture was stirred at room temperature for a further 48 hours. The reaction mixture was concentrated and the residue was purified by preparative HPLC (column: Chromatorex C18 10 μm, 250×30 mm, mobile phase A=water, B=acetonitrile; gradient: 0.0 min 5% B; 3 min 5% B; 20 min 50% B; 23 min 100% B; 26 min 5% B; flow rate: 50 ml/min; 0.1% formic acid). The product-containing fractions were combined, concentrated and dried under high vacuum. This gave 58 mg (97% pure, 38% of theory) of the title compound.

LC-MS (Method 2): Rₜ=0.73 min; MS (ESIpos): m/z=404 [M−HCO₂]⁺

¹H-NMR (400 MHz, DMSO-d6) δ [ppm]: −0.008 (1.39), 0.008 (1.29), 1.360 (15.93), 1.376 (16.00), 2.150 (0.41), 2.857 (5.60), 3.717 (1.83), 3.729 (1.87), 4.330 (2.19), 4.363 (1.18), 4.379 (1.46), 4.396 (1.07), 4.412 (0.41), 6.642 (4.11), 6.647 (4.11), 6.835 (0.97), 6.852 (1.67), 6.866 (0.92), 7.326 (1.13), 7.343 (1.20), 7.733 (3.20), 7.737 (3.09), 7.964 (4.28), 8.110 (1.09), 8.128 (1.10), 8.196 (2.04), 8.214 (1.72), 8.232 (1.53), 8.309 (1.25), 8.327 (1.18), 8.561 (2.45).

### Example 26

### 2-[({[3-(2-Methoxypyridin-3-yl)imidazo[1,2-a]pyridin-7-yl]carbonyl}amino)methyl]-1-methylimidazo[1,2-a]pyridin-1-ium formate

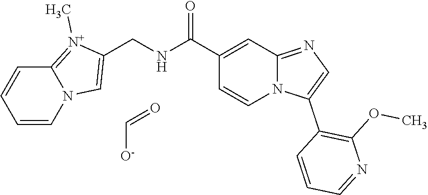

2-({[(3-Iodoimidazo[1,2-a]pyridin-7-yl)carbonyl]amino}methyl)-1-methylimidazo[1,2-a]pyridin-1-ium (120 mg, 278 μmol), (2-methoxypyridin-3-yl)boric acid (84.9 mg, 555 μmol), potassium carbonate (115 mg, 833 μmol) and [1,1-bis(diphenylphosphino)ferrocene]dichloropalladium(II) (20.3 mg, 27.8 μmol) were initially charged under argon. 3.5 ml of degassed dioxane/water (4:1) were added and the mixture was stirred at 90° C. for one hour. The reaction mixture was diluted with methanol, and 0.2 ml of formic acid was added. The mixture was filtered and the filtrate was purified by preparative HPLC (column: RP, Chromatorex C18, 250×30 mm 10 μm; flow rate: 50 ml/min; mobile phase: A=water+0.1% formic acid, B=acetonitrile; gradient: 0 min 5% B, 9 min 5% B, 24 min 95% B, 27 min 95% B, 29 min 10% B; detection: 210 nm). The product-containing fractions were combined, concentrated and dried under high vacuum. This gave 42 mg (100% pure, 33% of theory) of the title compound.

LC-MS (Method 2): Rₜ=0.71 min; MS (ESIpos): m/z=413 [M−HCO₂]⁺

¹H-NMR (400 MHz, DMSO-d6) δ [ppm]: 3.366 (2.73), 3.904 (16.00), 4.068 (12.78), 4.846 (2.26), 4.859 (2.22), 7.187 (1.47), 7.200 (1.55), 7.206 (1.54), 7.218 (1.52), 7.402 (1.25), 7.420 (1.29), 7.521 (0.80), 7.539 (1.59), 7.555 (0.88), 7.890 (1.49), 7.927 (1.59), 7.931 (1.70), 7.945 (1.56), 7.950 (1.49), 8.009 (0.79), 8.030 (1.11), 8.048 (0.90), 8.188 (1.00), 8.205 (2.59), 8.228 (1.37), 8.319 (1.97), 8.345 (1.66), 8.350 (1.67), 8.358 (1.63), 8.362 (1.52), 8.421 (2.31), 8.887 (1.35), 8.903 (1.30), 9.717 (0.56).

### Example 27

### 1-[2-({[3-(2-Methoxypyridin-3-yl)imidazo[1,2-a]pyridin-7-yl]carbonyl}amino)ethyl]-3-methyl-4-(methylamino)pyridinium formate

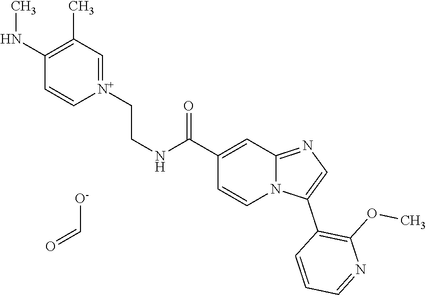

3-(2-Methoxypyridin-3-yl)imidazo[1,2-a]pyridine-7-carboxylic acid (60.0 mg, 80% pure, 178 μmol) and 1-(2-aminoethyl)-3-methyl-4-(methylamino)pyridinium chloride hydrochloride (1:1:1) (42.3 mg, 178 μmol) were initially charged in 1.9 ml of dichloromethane, 1-(3-dimethylaminopropyl)-3-ethylcarbodiimide hydrochloride (51.1 mg, 267 μmol) and 4-dimethylaminopyridine (65.1 mg, 533 μmol) were added and the mixture was stirred at room temperature overnight. The reaction mixture was concentrated and the residue was purified by preparative HPLC (column: Chromatorex C18 10 μm, 125×40 mm, mobile phase A=water, B=acetonitrile; gradient: 0.0 min 5% B; 3 min 5% B; 20 min 50% B; 23 min 100% B; 26 min 5% B; flow rate: 50 ml/min; 0.1% formic acid). The product-containing fractions were combined, concentrated and dried under high vacuum. This gave 17 mg (100% pure, 21% of theory) of the title compound.

LC-MS (Method 2): Rₜ=0.72 min; MS (ESIneg): m/z=415 [M−2H-HCO₂]⁺

¹H-NMR (400 MHz, DMSO-d6) δ [ppm]: −0.008 (1.14), 0.008 (0.95), 2.080 (9.55), 2.096 (0.52), 2.151 (1.89), 2.328 (0.45), 2.523 (1.12), 2.670 (0.46), 2.912 (5.24), 2.923 (5.18), 2.941 (0.68), 3.355 (1.82), 3.727 (1.52), 3.739 (1.54), 3.799 (1.03), 3.900 (16.00), 4.318 (1.23), 4.332 (1.79), 4.344 (1.14), 6.845 (1.89), 6.863 (1.91), 7.182 (1.48), 7.194 (1.56), 7.200 (1.54), 7.213 (1.55), 7.251 (1.34), 7.256 (1.32), 7.269 (1.31), 7.274 (1.37), 7.865 (5.03), 7.911 (1.61), 7.916 (1.66), 7.929 (1.57), 7.934 (1.51), 7.966 (0.78), 7.977 (0.76), 8.139 (4.14), 8.158 (1.82), 8.214 (2.24), 8.271 (1.15), 8.289 (1.12), 8.341 (1.53), 8.346 (1.55), 8.353 (1.52), 8.358 (1.38), 8.522 (1.17), 9.012 (0.75).

### Example 28

### 1-[2-({[3-(2-Methoxypyridin-3-yl)imidazo[1,2-a]pyridin-7-yl]carbonyl}amino)ethyl]-2-methyl-4-(methylamino)pyridinium formate

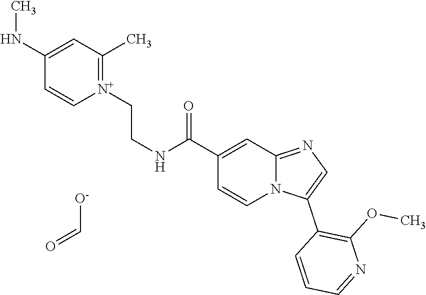

3-(2-Methoxypyridin-3-yl)imidazo[1,2-a]pyridine-7-carboxylic acid (60.0 mg, 80% pure, 178 μmol) and 1-(2-aminoethyl)-2-methyl-4-(methylamino)pyridinium chloride hydrochloride (1:1:1) (42.3 mg, 178 μmol) were initially charged in 1.9 ml of dichloromethane and 2 ml of dimethylformamide, 1-(3-dimethylaminopropyl)-3-ethylcarbodiimide hydrochloride (51.1 mg, 267 μmol) and 4-dimethylaminopyridine (65.1 mg, 533 μmol) were added and the mixture was stirred at room temperature overnight. More 1-(2-aminoethyl)-2-methyl-4-(methylamino)pyridinium chloride hydrochloride (1:1:1) (21 mg, 90 μmol), 4-dimethylaminopyridine (32.6 mg, 265 μmol) and 1-(3-dimethylaminopropyl)-3-ethylcarbodiimide hydrochloride (26 mg, 135 μmol) were added and the mixture was again stirred at room temperature overnight. The reaction mixture was then stirred at 40° C. for three hours. Subsequently, the reaction mixture was concentrated and the residue was purified by preparative HPLC (column: Chromatorex C18 10 μm, 125×40 mm, mobile phase A=water, B=acetonitrile; gradient: 0.0 min 5% B; 3 min 5% B; 20 min 50% B; 23 min 100% B; 26 min 5% B; flow rate: 50 ml/min; 0.1% formic acid). The product-containing fractions were combined, concentrated and dried under high vacuum. This gave 11.7 mg (95% pure, 14% of theory) of the title compound.

LC-MS (Method 2): Rₜ=0.65 min; MS (ESIpos): m/z=417 [M−HCO₂]⁺

¹H-NMR (400 MHz, DMSO-d6) δ [ppm]: 0.925 (0.41), 1.919 (0.57), 2.154 (1.41), 2.611 (5.38), 2.834 (1.98), 2.846 (2.36), 2.854 (3.19), 2.867 (2.99), 3.395 (0.62), 3.689 (1.71), 3.703 (1.76), 3.903 (16.00), 4.325 (1.67), 4.339 (1.47), 6.715 (1.14), 6.724 (1.17), 6.764 (0.42), 6.815 (1.07), 7.183 (1.47), 7.195 (1.58), 7.201 (1.58), 7.214 (1.52), 7.265 (1.21), 7.284 (1.24), 7.872 (5.12), 7.912 (1.63), 7.917 (1.74), 7.931 (1.55), 7.935 (1.59), 8.011 (0.95), 8.029 (0.92), 8.153 (4.06), 8.171 (1.79), 8.206 (0.67), 8.225 (0.55), 8.343 (1.69), 8.347 (1.75), 8.355 (1.68), 8.360 (1.51), 8.446 (2.83), 8.631 (0.64), 9.078 (0.90).

### Example 29

### 4-tert-Butyl-1-[2-({[3-(3,5-dimethyl-1,2-oxazol-4-yl)imidazo[1,2-a]pyridin-7-yl]carbonyl}amino)ethyl]pyridinium formate formic acid (1:1:1)

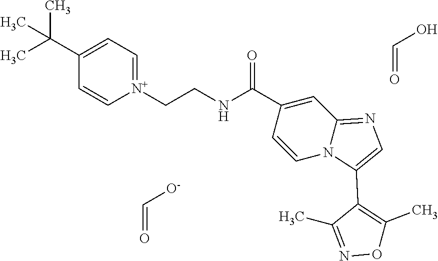

3-(3,5-Dimethyl-1,2-oxazol-4-yl)imidazo[1,2-a]pyridine-7-carboxylic acid (50.0 mg, 194 μmol) was initially charged in 2 ml of dichloromethane, 1-(2-ammonioethyl)-4-tert-butylpyridinium dibromide (66.1 mg, 194 μmol), 1-(3-dimethylaminopropyl)-3-ethylcarbodiimide hydrochloride (55.9 mg, 292 μmol) and 4-dimethylaminopyridine (95.0 mg, 777 μmol) were added and the mixture was stirred at room temperature for 48 hours. The reaction mixture was concentrated and purified by preparative HPLC (column: Chromatorex C18 10 μm, 250×30 mm, mobile phase A=water, B=acetonitrile; gradient: 0.50 min 5% B; 3 min 5% B; 20 min 50% B; 23 min 100% B; 26 min 5% B; flow rate: 50 ml/min; 0.1% formic acid). The product-containing fractions were combined, concentrated and dried under high vacuum. This gave 58 mg (100% pure, 59% of theory) of the title compound.

LC-MS (Method 2): Rₜ=0.85 min; MS (ESIpos): m/z=418 [M−HCO₂-HCO₂H]⁺

¹H-NMR (400 MHz, DMSO-d6) δ [ppm]: 1.345 (16.00), 2.111 (5.51), 2.319 (5.56), 3.865 (0.60), 3.879 (0.61), 4.748 (0.66), 7.216 (0.47), 7.234 (0.48), 7.867 (1.44), 8.122 (0.88), 8.141 (1.12), 8.159 (1.09), 8.227 (0.61), 8.245 (0.58), 8.345 (0.88), 8.964 (0.95), 8.981 (0.93).

### Example 30

### 1-[2-({[3-(3,5-Dimethyl-1,2-oxazol-4-yl)imidazo[1,2-a]pyridin-7-yl]carbonyl}amino)ethyl]-4-isopropylpyridinium formate

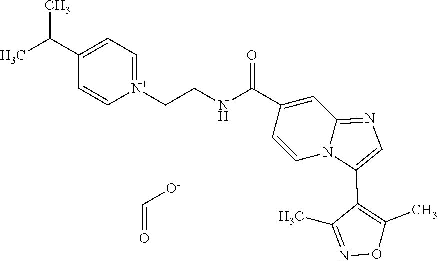

3-(3,5-Dimethyl-1,2-oxazol-4-yl)imidazo[1,2-a]pyridine-7-carboxylic acid (50.0 mg, 194 μmol) was initially charged in 2 ml of dichloromethane, 1-(2-ammonioethyl)-4-isopropylpyridinium dibromide (63.4 mg, 194 μmol), 1-(3-dimethylaminopropyl)-3-ethylcarbodiimide hydrochloride (55.9 g, 292 mmol) and dimethylaminopyridine (95.0 mg, 777 μmol) were added and the mixture was stirred at room temperature for 48 hours. The reaction mixture was concentrated and the residue was purified by preparative HPLC (column: Chromatorex C18 10 μm, 250×30 mm, mobile phase A=water, B=acetonitrile; gradient: 0.0 min 5% B; 3 min 5% B; 20 min 50% B; 23 min 100% B; 26 min 5% B; flow rate: 50 ml/min; 0.1% formic acid). The product-containing fractions were combined, concentrated and dried under high vacuum. This gave 14.5 mg (100% pure, 17% of theory) of the title compound.

LC-MS (Method 2): Rₜ=0.77 min; MS (ESIpos): m/z=404 [M−HCO₂]⁺

¹H-NMR (400 MHz, DMSO-d6) δ [ppm]: 1.248 (13.24), 1.265 (13.42), 2.111 (16.00), 2.319 (15.81), 3.172 (0.84), 3.189 (1.11), 3.206 (0.85), 3.224 (0.42), 3.339 (1.20), 3.871 (1.63), 3.883 (1.67), 4.755 (1.77), 7.238 (1.16), 7.256 (1.19), 7.861 (3.52), 8.031 (2.65), 8.047 (2.78), 8.149 (2.11), 8.212 (1.43), 8.230 (1.38), 8.555 (1.67), 8.998 (2.03), 9.012 (1.66).

### Example 31

### 1-[2-({[3-(4-Methoxypyridin-3-yl)imidazo[1,2-a]pyridin-7-yl]carbonyl}amino)ethyl]-4-(methylamino)pyridinium bromide

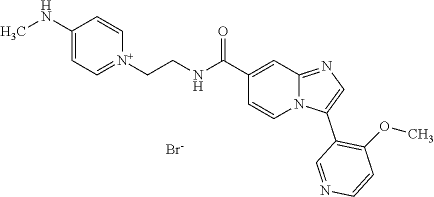

1-(2-{[(3-Iodoimidazo[1,2-a]pyridin-7-yl)carbonyl]amino}ethyl)-4-(methylamino)pyridinium bromide (70.0 mg, 139 μmol), (4-methoxypyridin-3-yl)boric acid (42.6 mg, 279 μmol), potassium carbonate (57.8 mg, 418 μmol) and [1,1-bis(diphenylphosphino)ferrocene]dichloropalladium(II) (10.2 mg, 13.9 μmol) were initially charged under argon. 2 ml of degassed dioxane/water (4:1) were added and the mixture was stirred at 90° C. for three hours. The reaction mixture was diluted with methanol, 0.5 ml of formic acid was added and the mixture was filtered. The filtrate was purified by preparative HPLC (column: Chromatorex C18 10 μm, 125×40 mm, mobile phase A=water, B=acetonitrile; gradient: 0.0 min 10% B; 5 min 10% B; 19 min 50% B; 20 min 95% B; 26 min 10% B; flow rate: 100 ml/min; 0.1% formic acid). The product-containing fractions were combined and concentrated by evaporation. The residue was re-purified by preparative TLC (Alox neutral, mobile phase: dichloromethane/methanol 10:1). This gave 22.1 mg (95% pure, 31% of theory) of the title compound.

¹H-NMR (500 MHz, DMSO-d6) δ [ppm]: 0.006 (1.46), 2.869 (15.13), 3.714 (1.71), 3.721 (1.74), 3.884 (16.00), 4.313 (1.66), 4.323 (2.29), 4.334 (1.53), 6.833 (1.16), 6.847 (1.39), 6.856 (1.40), 6.870 (1.19), 7.239 (1.62), 7.243 (1.65), 7.254 (1.64), 7.257 (1.69), 7.309 (2.77), 7.321 (2.87), 7.872 (6.30), 8.102 (3.49), 8.116 (3.37), 8.149 (3.08), 8.300 (1.17), 8.315 (1.18), 8.517 (6.03), 8.613 (3.43), 8.624 (3.28), 8.893 (0.83).

### Example 32

### 1-[2-({[3-(3,5-Dimethyl-1,2-oxazol-4-yl)imidazo[1,2-a]pyridin-7-yl]carbonyl}amino)ethyl]-4-ethylpyridinium formate

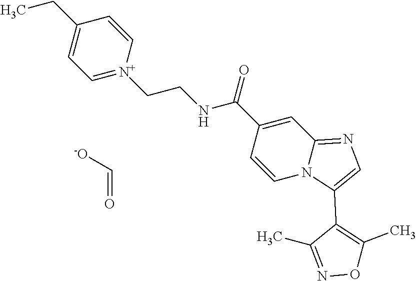

3-(3,5-Dimethyl-1,2-oxazol-4-yl)imidazo[1,2-a]pyridine-7-carboxylic acid (50.0 mg, 194 μmol) was initially charged in 2 ml of dichloromethane, 1-(2-ammonioethyl)-4-ethylpyridinium dibromide (60.7 mg, 194 μmol), 1-(3-dimethylaminopropyl)-3-ethylcarbodiimide hydrochloride (55.9 g, 292 mmol) and dimethylaminopyridine (95.0 mg, 777 μmol) were added and the mixture was stirred at room temperature for 48 hours. The reaction mixture was concentrated and the residue was purified by preparative HPLC (column: Chromatorex C18 10 μm, 250×30 mm, mobile phase A=water, B=acetonitrile; gradient: 0.0 min 5% B; 3 min 5% B; 20 min 50% B; 23 min 100% B; 26 min 5% B; flow rate: 50 ml/min; 0.1% formic acid). The product-containing fractions were combined, concentrated and dried under high vacuum. This gave 22 mg (100% pure, 26% of theory) of the title compound.

LC-MS (Method 2): Rₜ=0.71 min; MS (ESIpos): m/z=390 [M−HCO₂]⁺

¹H-NMR (400 MHz, DMSO-d6) δ [ppm]: 1.221 (3.25), 1.239 (6.82), 1.258 (3.45), 2.111 (15.76), 2.146 (0.47), 2.319 (16.00), 2.864 (1.06), 2.882 (2.98), 2.901 (2.92), 2.920 (1.02), 3.015 (0.47), 3.868 (2.24), 3.880 (2.29), 4.747 (2.38), 7.240 (1.51), 7.257 (1.54), 7.862 (3.91), 7.991 (3.24), 8.007 (3.27), 8.148 (2.56), 8.210 (1.73), 8.228 (1.64), 8.507 (1.84), 8.978 (2.58).

### Example 33

### 1-[2-({[3-(3,5-Dimethyl-1,2-oxazol-4-yl)imidazo[1,2-a]pyridin-7-yl]carbonyl}amino)ethyl]-3-phenoxypyridinium formate

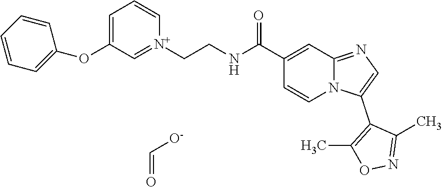

Sodium 3-(3,5-dimethyl-1,2-oxazol-4-yl)imidazo[1,2-a]pyridine-7-carboxylate (100 g, 358 μmol) and 1-(2-aminoethyl)-3-phenoxypyridinium bromide (117 mg, 394 μmol) were initially charged in 3 ml of dichloromethane, 1-(3-dimethylaminopropyl)-3-ethylcarbodiimide hydrochloride (103 mg, 537 μmol) and 4-dimethylaminopyridine (131 g, 1.07 mmol) were added and the mixture was stirred at room temperature for four hours. The reaction mixture was dissolved in water/acetonitrile and purified by preparative HPLC (column: Chromatorex C18 10 μm, 250×30 mm, mobile phase A=water, B=acetonitrile; gradient: 0.0 min 5% B; 3 min 5% B; 20 min 50% B; 23 min 100% B; 26 min 5% B; flow rate: 50 ml/min; 0.1% formic acid). The product-containing fractions were combined, concentrated and dried under high vacuum. This gave 103 mg (99% pure, 57% of theory) of the title compound.

LC-MS (Method 2): Rₜ=0.88 min; MS (ESIpos): m/z=454 [M−HCO₂]⁺

¹H-NMR (400 MHz, DMSO-d6) δ [ppm]: 2.112 (15.97), 2.321 (16.00), 3.387 (0.72), 3.893 (1.65), 3.903 (1.60), 4.801 (1.86), 7.124 (3.00), 7.143 (3.62), 7.210 (0.75), 7.229 (1.87), 7.247 (1.21), 7.270 (1.38), 7.287 (1.39), 7.355 (2.45), 7.375 (3.12), 7.394 (1.58), 7.881 (5.10), 8.086 (0.90), 8.102 (0.96), 8.108 (1.12), 8.123 (1.08), 8.177 (2.39), 8.233 (1.89), 8.251 (2.72), 8.266 (0.93), 8.510 (5.33), 8.917 (1.26), 8.931 (1.18), 9.130 (1.90), 9.364 (0.40).

### Example 34

### 4-(Methylamino)-1-[2-({[3-(2-methylpyridin-3-yl)imidazo[1,2-a]pyridin-7-yl]carbonyl}amino)ethyl]pyridinium formate

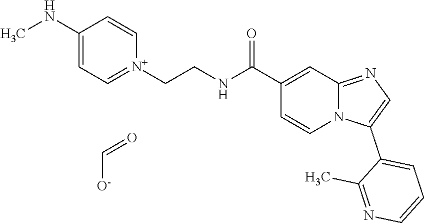

1-(2-{[(3-Iodoimidazo[1,2-a]pyridin-7-yl)carbonyl]amino}ethyl)-4-(methylamino)pyridinium bromide (70.0 mg, 139 μmol), (2-methylpyridin-3-yl)boric acid (38.2 mg, 279 μmol), potassium carbonate (57.8 mg, 418 μmol) and [1,1-bis(diphenylphosphino)ferrocene]dichloropalladium(II) (10.2 mg, 13.9 μmol) were initially charged under argon. 2 ml of degassed dioxane/water (1:1) were added and the mixture was stirred at 90° C. for 1.5 hours. The reaction mixture was diluted with methanol, 0.5 ml of formic acid was added and the mixture was filtered. The filtrate was purified by preparative HPLC (column: Chromatorex C18 10 μm, 125×40 mm, mobile phase A=water, B=acetonitrile; gradient: 0.0 min 10% B; 5 min 10% B; 19 min 50% B; 20 min 95% B; 26 min 10% B; flow rate: 100 ml/min; 0.1% formic acid). The product-containing fractions were combined, concentrated and dried under high vacuum. The residue was re-purified by preparative TLC (Alox neutral, mobile phase: dichloromethane/methanol 10:1). This gave 21.3 mg (90% pure, 32% of theory) of the title compound.

¹H-NMR (400 MHz, DMSO-d6) δ [ppm]: 2.309 (1.58), 2.324 (15.42), 2.868 (16.00), 3.718 (2.12), 4.313 (1.85), 4.326 (2.66), 4.339 (1.71), 6.834 (1.42), 6.850 (3.40), 6.866 (1.72), 7.262 (1.84), 7.266 (1.96), 7.280 (1.89), 7.284 (2.02), 7.408 (1.25), 7.420 (1.35), 7.427 (1.40), 7.439 (1.39), 7.837 (1.72), 7.841 (1.84), 7.856 (1.63), 7.860 (1.65), 7.890 (4.98), 7.908 (0.55), 8.103 (3.22), 8.121 (2.99), 8.182 (3.68), 8.298 (1.48), 8.317 (1.58), 8.614 (1.66), 8.618 (1.75), 8.626 (1.69), 8.630 (1.66), 8.911 (0.77).

### Example 35

### 1-[2-({[3-(3,5-Dimethyl-1,2-oxazol-4-yl)imidazo[1,2-a]pyridin-7-yl]carbonyl}amino)ethyl]-4-(piperidin-1-yl)pyridinium formate

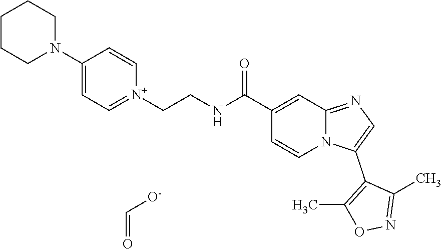

Sodium 3-(3,5-dimethyl-1,2-oxazol-4-yl)imidazo[1,2-a]pyridine-7-carboxylate (100 mg, 358 μmol) and 1-(2-ammonioethyl)-4-(piperidin-1-yl)pyridinium dibromide (145 mg, 394 μmol) were initially charged in 2 ml of dichloromethane. 1-(3-Dimethylaminopropyl)-3-ethylcarbodiimide hydrochloride (103 mg, 537 μmol) and 4-dimethylaminopyridine (131 mg, 1.07 mmol) were added and the mixture was stirred at room temperature overnight. Subsequently, the reaction mixture was concentrated and the residue was purified by preparative HPLC (column: Chromatorex C18 10 μm, 250×30 mm, mobile phase A=water, B=acetonitrile; gradient: 0.0 min 5% B; 3 min 5% B; 20 min 50% B; 23 min 100% B; 26 min 5% B; flow rate: 50 ml/min; 0.1% formic acid). The product fractions were combined, concentrated and lyophilized. This gave 39.8 mg (89% pure, 20% of theory) of the title compound.

LC-MS (Method 2): Rₜ=0.92 min; MS (ESIpos): m/z=445 [M−HCO₂]⁺

¹H-NMR (500 MHz, DMSO-d6) δ [ppm]: 1.553 (2.64), 1.560 (2.26), 1.636 (0.62), 1.649 (1.26), 1.659 (1.34), 2.114 (16.00), 2.323 (14.93), 3.627 (2.68), 3.638 (3.37), 3.648 (2.62), 3.743 (1.30), 3.752 (1.31), 4.364 (1.09), 4.374 (1.55), 7.182 (2.05), 7.197 (2.09), 7.310 (1.18), 7.325 (1.33), 7.848 (1.27), 7.862 (0.8).

### B. ASSESSMENT OF PHARMACOLOGICAL EFFICACY

B1 In-Vitro Determination of the Antagonistic Action

Antagonism against the α2B adrenoreceptor (ADRA2B) was tested using a recombinant human α2B-Gα16 receptor fusion protein CHO cell line which additionally also recombinantly expresses the photoprotein mitochondrial obelin.

The cells were cultivated at 37° C. and 5% CO2 in Dulbecco's modified Eagle's Medium/NUT mix F12 with L-glutamine which additionally contains 10% (v/v) inactivated foetal calf serum, 1 mM sodium pyruvate, 0.9 mM sodium bicarbonate, 50 U/ml penicillin, 50 μg/ml streptomycin, 2.5 μg/ml amphotericin B and 1 mg/ml Geneticin. The cells were passaged with enzyme-free Hank's-based cell dissociation buffer. All cell culture reagents used were from Invitrogen (Carlsbad, USA).

Luminescence measurements were carried out on white 384-well microtitre plates. 2000 cells/well were plated in a volume of 25 μl and cultivated for one day at 30° C. and 5% CO2 in cell culture medium with coelenterazine (α2B: 5 μg/ml). Serial dilutions of the test substances (10 μl) were added to the cells. After 6 minutes, noradrenaline was added to the cells (35 μl; final concentration: EC50-EC80), and the emitted light was measured for 50 seconds using a CCD (charge-coupled device) camera (Hamamatsu Corporation, Shizuoka, Japan) in a light-tight box.

The test substances were tested up to a maximum concentration of 10 μM. The IC50 values (shown in Table 1) were calculated from the appropriate dose-response curves.

TABLE 1

| Example No. | IC₅₀ [nM] |
|---|---|
| 1 | 9 |
| 2 | 19 |
| 3 | 18 |
| 4 | 24 |
| 5 | 13 |
| 6 | 29 |
| 7 | 55 |
| 8 | 57 |
| 9 | 8 |
| 10 | 165 |
| 11 | 100 |
| 12 | 370 |
| 13 | 200 |
| 14 | 815 |
| 15 | 28 |
| 16 | 36 |
| 17 | 49 |
| 18 | 86 |
| 19 | 205 |
| 20 | 225 |
| 21 | 265 |
| 22 | 275 |
| 23 | 330 |
| 24 | 510 |
| 25 | 545 |
| 26 | 4 |
| 27 | 11 |
| 28 | 19 |
| 29 | 47 |
| 30 | 66 |
| 31 | 86 |
| 32 | 94 |
| 33 | 155 |
| 34 | 785 |
| 35 | 470 |

B2. Determination of Coronary Flow Reserve in the Anaesthetized Dog

Haemodynamic studies on anaesthetized and analgized dogs may be carried out to assess the in vivo efficacy of the substances.

To this end, anaesthesia is induced using pentobarbital sodium and pancuronium bromide, and is maintained using pentobarbital sodium, fentanyl and an ambient air/oxygen mix. Additionally, Ringer lactate solution is infused.

The later determination of the coronary flow reserve requires quantification of the coronary blood flow. This can be effected with flow meter probes placed around the coronary vessels.

Following intravenous or intracoronary administration of a dilator such as adenosine (generally 140 μg/kg/min for 5 min as infusion), the increase in coronary blood flow as a response of adenosine can be measured using the flow meter probes.

Comparison of “coronary flow during the administration of adenosine” (e.g. peak flow during adenosine infusion) to the “basal flow” (mean flow of generally 3 min prior to the adenosine infusion) allows a statement to be made about the coronary flow reserve, i.e. the maximum amount of blood volume which, under stress, can be provided in addition to the basal flow for supplying the heart muscle. The coronary flow reserve (peak flow under adenosine/basal flow) can be determined from these measurements.

Subsequently, L-NAME (generally 60 μg/kg/min at 15 μl/kg/min for 60 min as continuous infusion) is infused to the dogs inter alia for blocking endothelial NO synthase to mimic endothelial damage.

With further continuous infusion of L-NAME, administration of adenosine—as described above—is then repeated to determine the reduction of the coronary flow reserve as a result of L-NAME infusion (blockade of NO synthase). Finally, with further continuous infusion of L-NAME, the effects on the coronary flow reserve (adenosine infusion as described above) are then determined after vehicle administration and subsequent administration of the ADRA2b antagonists. Vehicle and ADRA2b antagonist are administered intravenously as “bole (50 μl/kg)+infusion (infusion rate: 450 μl/kg/h)”.

B3 Determination of the Infarct Size in the Rat

To assess the in vivo efficacy of the substances, it is possible to determine the effect of a substance on the size of the infarct area (based on the hypoperfused area at risk) in the rat, as well as haemodynamic parameters of cardiac function. To this end, substance-treated animals were compared to animals which had received placebo only. In principle, the method of acute myocardial infarction in the rat consists of a surgical procedure (under anaesthesia and analgesia) where a coronary artery, preferably the LAD (left anterior descending artery), is ligated with a suture and, after a defined occlusion phase of 30 min, opened again. After this time, the vessel is re-opened by removing the suture (reperfusion of cardiac tissue). The thorax of the animal is closed again, and the muscle layers and the epidermis are sutured using suture material (Vicryl L 4-0 or 5-0 (V990H)). In a final examination under anaesthesia and analgesia, the animal is fitted with instruments (introduction of a Millar catheter (2 F) via the carotid artery to measure heart haemodynamics). At the end of the measurements, the animals are, without having woken, sacrificed painlessly using an overdose of anaesthetics (isofluran >5%, pentobarbital >200 mg/kg) and/or exsanguination under deep anaesthesia. Determination of the area at risk (non-perfused area) and the infacrt size in the heart are carried outpost mortem by perfusion with Evans Blue (0.2%) to determine the regions not perfused as a result of the occlusion (area at risk) and subsequent detection of vital tissue by TTC stain (triphenyltetrazolium chloride (TTC), (vital stain).

B4 Haemodynamic Studies

Haemodynamic studies on rats may be carried out to assess the in vivo efficacy of the substances. To this end, rats (WiWu strain) are pretreated with reserpine (5 mg/kg s.c.) for 3 days. This results in an enhanced effect of adrenergic agonists and antagonists in the animals. In the rats pretreated in this manner, blood pressure is measured invasively under anaesthesia. Initially, the animals are administered an antagonist (i.v.), followed by i.v. administration of the ADRA2 agonist dexmedetomidine 3 μg/kg/min (15 min). Selective ADRA2b antagonists counteract an agonist-induced blood pressure increase in a dose-dependent manner.

B5 PK Assay

iv (Intravenous) Studies:

To examine the pharmacokinetic properties of the substances, the substances in question can be administered to animals (e.g. rats, dogs) as a bole or an infusion. Preferably, the substances are formulated in 0.9% strength saline, plasma/dimethyl sulfoxide (99/1), polyethylene glycol/ethanol/water in a ratio of 50/10/40 (other suitable formulation agents are also possible).

Blood samples may be removed from the animals via a catheter or venipuncture and be collected in anticoagulant-containing (e.g. lithium heparinate or potassium EDTA) tubes. At the following points in time, blood samples are taken from the test animals: 0.033, 0.083, 0.167, 0.25, 0.283, 0.333, 0.5, 0.75, 1, 2, 3, 5, 7, 24 hours after substance administration. (Removal of fewer samples or further samples at later points in time is also possible.) To obtain plasma, the blood samples are centrifuged. The supernatant (plasma) is taken off and either directly processed further or frozen for later sample preparation. For sample preparation, 50 μl of plasma are mixed with 250 μl of acetonitrile (the precipitating agent acetonitrile also contains the internal standard ISTD for later analytical determination) and then allowed to stand at room temperature for 5 minutes. The mixture is then centrifuged at 16 000 g for 3 minutes. The supernatant is taken off, and 500 μl of a buffer suitable for the mobile phase are added. The samples are then examined by LC-MS/MS analysis (e.g. liquid chromatography using a Gemini 5 μM C18 110A 50 mm×3 mm (or 150 mm×3 mm) column from Phenomenex; by mass spectrometry using an API 5500 or API 6500; SCIEX, Canada) to determine the concentration of the substance in the individual samples.

In addition to the the plasma concentrations, the concentration ratio whole blood to plasma for the substance in question may also be determined. To this end, the substance is incubated at a certain concentration in whole blood for 20 minutes. The samples are then processed as described above to determine the concentration of the substance in the plasma. The concentration set divided by the concentration measured in the plasma gives the parameter Cb/Cp.

The pharmacokinetic parameters are calculated by non-compartmental analysis (NCA). The algorithms for calculating the parameters are based on rules published in general textbooks of pharmacokinetics (e.g. Rowland and Tozer, Clinical Pharmacokinetics and Pharmacodynamics, ISBN 978-0-7817-5009-7).

The primary pharmacokinetic parameters clearance (CL) and distribution volume (Vss) can be calculated as follows:

| Parameter | Formula |
|---|---|
| CLplasma | CLplasma = dose/AUC |
| (Plasma Clearance) | (AUC = area under the curve) |
| CLblood | CLblood = CLplasma/(Cb/Cp) |
| (blood clearance) | |
| Vss | Vss = CLplasma * MRTiv |
| MRTiv | MRTiv = AUMC/AUC |
| AUMC | AUMC = AUMC(0-tₗₐₛₜ) + |
| | tₗₐₛₜ*C_{last,calculated}/λ_{z} + C_{last,calculated}/λ_{z}² |
| λ_{z} | Rate constant for the terminal phase; |
| | calculated from the logarithmic-linear |
| | regression of unweighted data from the terminal |
| | phase with data points above the detection limit |
| AUC | AUC = AUC(0-tlast) + C_{last,calculated}/λ_{z} |
| AUCnorm | AUC divided by the dosage |
| | normalized to body weight (mg/kg) |

### C. WORKING EXAMPLES OF PHARMACEUTICAL COMPOSITIONS

The compounds of the invention can be converted to pharmaceutical preparations as follows:

i.v. Solution:

The compound according to the invention is dissolved in a concentration below the saturation solubility in a physiologically tolerated solvent (e.g. isotonic saline, 5% glucose solution and/or 30% PEG 400 solution). The solution is sterilized by filtration and used to fill sterile and pyrogen-free injection containers.

1: A compound of formula (I) 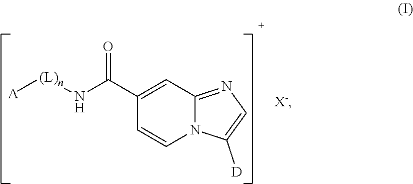
in which
A represents a positively charged aza heteroaromatic of the formula 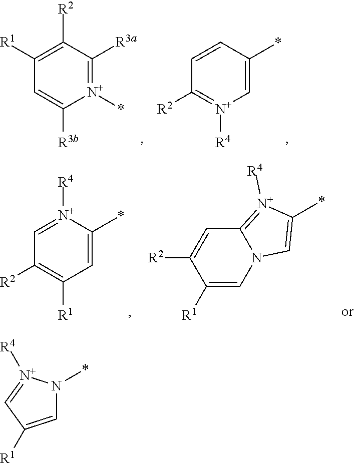
in which
* represents the point of attachment,
R¹, R², and R^{3a}, R^{3b} independently of one another represent a radical selected from the group consisting of hydrogen, amino, (C₁-C₄)-alkyl, (C₁-C₄)-alkoxy, mono-(C₁-C₄)-alkylamino, di-(C₁-C₄)-alkylamino, phenoxy and piperidin-1-yl,
where phenoxy and piperidin-1-yl may be substituted by (C₁-C₄)-alkyl and/or fluorine and
where the alkyl groups in (C₁-C₄)-alkyl, (C₁-C₄)-alkoxy, mono-(C₁-C₄)-alkylamino and di-(C₁-C₄)-alkylamino may each be up to pentasubstituted by fluorine,
R⁴ represents (C₁-C₄)-alkyl which may be up to pentasubstituted by fluorine, or represents a group of the formula CH₂CN, CH₂CONH₂,
D represents a heteroaromatic of the formula 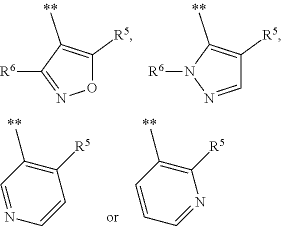
in which
** represents the point of attachment,
R⁵ and R⁶ independently of one another represent hydrogen, (C₁-C₄)-alkyl or (C₁-C₄)-alkoxy,
where (C₁-C₄)-alkyl and (C₁-C₄)-alkoxy may each be up to pentasubstituted by fluorine,
L represents CH₂,
n represents the number 0, 1, 2 or 3 and
X⁻ represents a physiologically acceptable anion,
or a solvate, a salt, or a solvate of the salt thereof.

2: The compound of formula (I) according to claim 1, in which
R¹, R², and R^{3a}, R^{3b} independently of one another represent a group selected from hydrogen, ethylamino, dimethylamino, methylamino, amino, methyl, ethyl, trifluoromethyl, t-butyl, isopropyl, phenoxy or piperidin-1-yl,
R⁴ represents methyl,
R⁵ and R⁶ independently of one another represent hydrogen, methyl, ethyl, isopropyl or methoxy,
n represents the number 1 or 2,
X⁻ represents bromide, chloride or formate, and
A represents a positively charged aza heteroaromatic of the formula 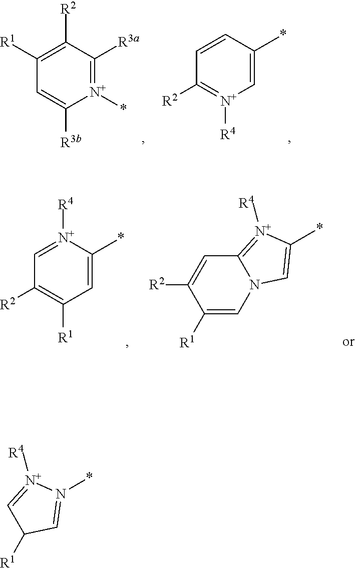
in which
* represents the point of attachment,
D represents a heteroaromatic of the formula 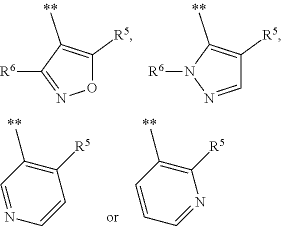
in which
** represents the point of attachment and
L represents CH₂
or a solvate, a salt, or a solvate of the salt thereof.

3: The compound of formula (I) according to claim 1, in which
R¹ represents hydrogen or methylamino,
R² represents hydrogen or methyl,
R^{3a}, R^{3b} represent hydrogen,
R⁴ represents methyl,
R⁵ and R⁶ independently of one another represent methyl, methoxy or hydrogen,
n represents the number 1 or 2,
X⁻ represents bromide, chloride or formate and
A represents a positively charged aza heteroaromatic of the formula 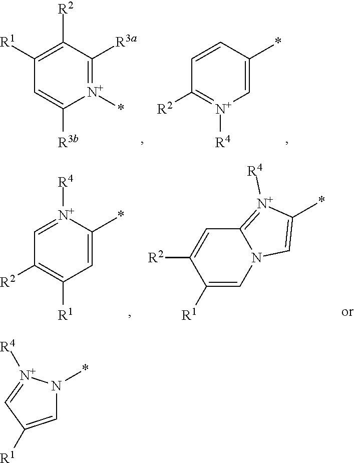
in which
*represents the point of attachment,
D represents a heteroaromatic of the formula 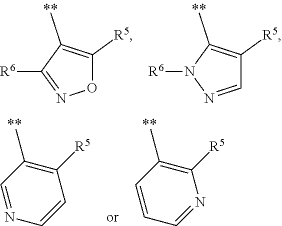
in which
** represents the point of attachment and
L represents CH₂,
or a solvate, a salt, or a solvate of the salt thereof.

4: The compound of formula (I) according to claim 1, wherein the compound is selected from the group consisting of
1-[2-({[3-(3,5-dimethyl-1,2-oxazol-4-yl)imidazo[1,2-a]pyridin-7-yl]carbonyl}amino)ethyl]-4-(methylamino)pyridinium chloride hydrochloride 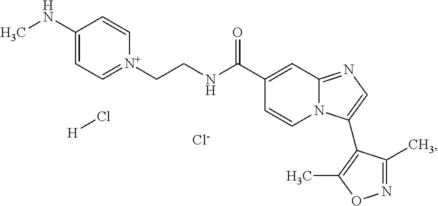 [SMILES: CNc1cc[n+](CCNC(=O)c2ccn3c(-c4c(C)noc4C)cnc3c2)cc1 ; Cl ; [Cl-]]
2-[({[3-(3,5-dimethyl-1,2-oxazol-4-yl)imidazo[1,2-a]pyridin-7-yl]carbonyl}amino)methyl]-1-methylimidazo[1,2-a]pyridin-1-ium formate 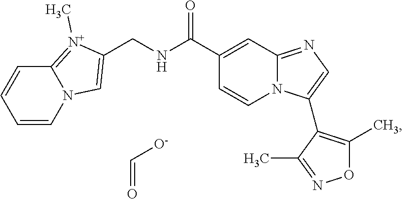
1-[2-({[3-(3,5-dimethyl-1,2-oxazol-4-yl)imidazo[1,2-a]pyridin-7-yl]carbonyl}amino)ethyl]-4-(methylamino)pyridinium formate 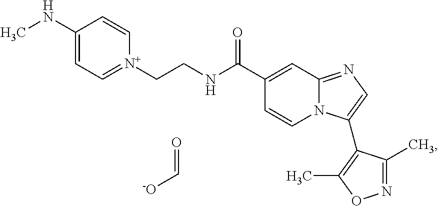
1-[2-({[3-(3,5-dimethyl-1,2-oxazol-4-yl)imidazo[1,2-a]pyridin-7-yl]carbonyl}amino)ethyl]-4-(methylamino)pyridinium chloride 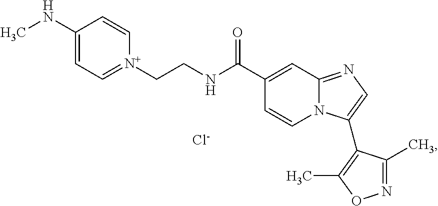 [SMILES: CNc1cc[n+](CCNC(=O)c2ccn3c(-c4c(C)noc4C)cnc3c2)cc1 ; [Cl-]]
1-[2-({[3-(1,4-dimethyl-1H-pyrazol-5-yl)imidazo[1,2-a]pyridin-7-yl]carbonyl}amino)ethyl]-4-(methylamino)pyridinium formate 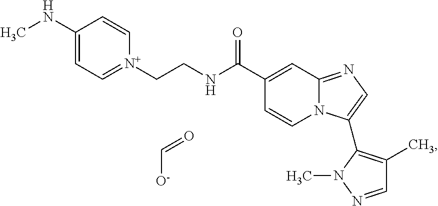
1-[2-({[3-(2-methoxypyridin-3-yl)imidazo[1,2-a]pyridin-7-yl]carbonyl}amino)ethyl]-4-(methylamino)pyridinium formate 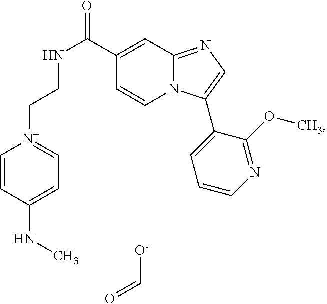
2-[({[3-(2-methoxypyridin-3-yl)imidazo[1,2-a]pyridin-7-yl]carbonyl}amino)methyl]-1-methylimidazo[1,2-a]pyridin-1-ium formate 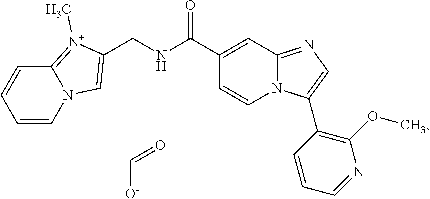
1-[2-({[3-(2-methoxypyridin-3-yl)imidazo[1,2-a]pyridin-7-yl]carbonyl}amino)ethyl]-3-methyl-4-(methylamino)pyridinium formate 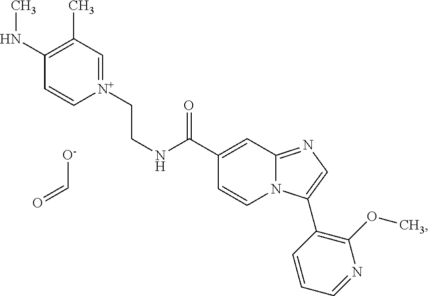
and
1-[2-({[3-(4-methoxypyridin-3-yl)imidazo[1,2-a]pyridin-7-yl]carbonyl}amino)ethyl]-4-(methylamino)pyridinium bromide 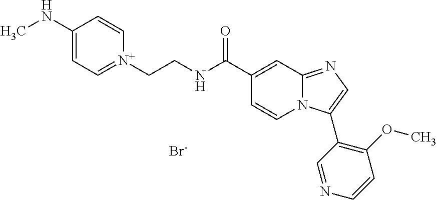
or a solvate, a salt, or a solvate of the salt thereof.

5: A process for preparing a compound of formula (I) according to claim 1, characterized in that
a compound of the formula (II) or its corresponding carboxylic acid 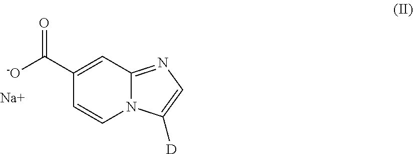
in which D has the meaning given above, is reacted in an inert solvent with a condensing agent such as, for example, 1-(3-dimethylaminopropyl)-3-ethylcarbodiimide hydrochloride in the presence of a base such as, for example, 4-dimethylaminopyridine with a compound of the formula (III)
A-(L)ₙ-NH₂ (III)
in which A, L and n have the meaning given above.

6: A method for treatment and/or prophylaxis of diseases, comprising administering an effective amount of a compound of formula (I) according to claim 1 to a human or animal in need thereof.

7: A method for treatment or prophylaxis of acute heart failure, right heart failure, left heart failure, global failure, diabetic heart failure, heart failure with preserved ejection fraction (HFpEF), diastolic heart failure, heart failure with reduced ejection fraction (HFrEF systolic heart failure), unstable angina pectoris, myocardial ischaemia, acute coronary syndrome, NSTEMI (non-ST elevation myocardial infarction), STEMI (ST elevation myocardial infarction), ischaemic heart muscle damage, myocardial infarction, coronary microvascular dysfunction, microvascular obstruction, no-reflow phenomenon, transitory and ischaemic attacks, ischaemic and haemorrhagic stroke, peripheral and cardial vascular disorders, impaired peripheral circulation, peripheral arterial occlusive disease, primary and secondary Raynaud's syndrome, impaired microcirculation, arterial pulmonary hypertension, spasms of coronary arteries and peripheral arteries, restenoses such as after thrombolysis therapy, percutaneous transluminal angioplasty (PTA), transluminal coronary angioplasty (PTCA), reperfusion damage, endothelial dysfunction, ischaemic cardiomyopathy, renal insufficiency, nephropathies and stress-related hypertension, comprising administering an effective amount of a compound of formula (I) according to claim 1 to a human or animal in need thereof.

8. (canceled)

9: A pharmaceutical composition comprising a compound according to claim 1 in combination with one or more inert, nontoxic, pharmaceutically suitable excipients.

10: A pharmaceutical combination comprising a compound according to claim 1 in combination with one or more active compounds selected from the group of the platelet aggregation inhibitors, anticoagulants, profibrinolytic substances, substances which affect the energy metabolism of the heart and mitochondrial function/ROS production, hypotensive drugs, mineralocorticoid receptor antagonists, HMG CoA reductase inhibitors, drugs which modulate lipid metabolism, active compounds which modulate glucose metabolism and active compounds for anxiety and pain therapy such as benzodiazepines and opiates.

11: A method for treatment or prophylaxis of acute heart failure, right heart failure, left heart failure, global failure, diabetic heart failure, heart failure with preserved ejection fraction (HFpEF), diastolic heart failure, heart failure with reduced ejection fraction (HFrEF systolic heart failure), coronary heart disease, stable and unstable angina pectoris, myocardial ischaemia, acute coronary syndrome, NSTEMI (non-ST elevation myocardial infarction), STEMI (ST elevation myocardial infarction), ischaemic heart muscle damage, myocardial infarction, coronary microvascular dysfunction, microvascular obstruction, no-reflow phenomenon, transitory and ischaemic attacks, ischaemic and haemorrhagic stroke, peripheral and cardial vascular disorders, impaired peripheral circulation, peripheral arterial occlusive disease, primary and secondary Raynaud's syndrome, impaired microcirculation, arterial pulmonary hypertension, spasms of coronary arteries and peripheral arteries, restenoses such as after thrombolysis therapy, percutaneous transluminal angioplasty (PTA), transluminal coronary angioplasty (PTCA), reperfusion damage, endothelial dysfunction, ischaemic cardiomyopathy, renal insufficiency, nephropathies and stress-related hypertension, comprising administering an effective amount of a pharmaceutical composition according to claim 9 to a human or animal in need thereof.

12: A method for treatment or prophylaxis of acute heart failure, right heart failure, left heart failure, global failure, diabetic heart failure, heart failure with preserved ejection fraction (HFpEF), diastolic heart failure, heart failure with reduced ejection fraction (HFrEF systolic heart failure), coronary heart disease, stable and unstable angina pectoris, myocardial ischaemia, acute coronary syndrome, NSTEMI (non-ST elevation myocardial infarction), STEMI (ST elevation myocardial infarction), ischaemic heart muscle damage, myocardial infarction, coronary microvascular dysfunction, microvascular obstruction, no-reflow phenomenon, transitory and ischaemic attacks, ischaemic and haemorrhagic stroke, peripheral and cardial vascular disorders, impaired peripheral circulation, peripheral arterial occlusive disease, primary and secondary Raynaud's syndrome, impaired microcirculation, arterial pulmonary hypertension, spasms of coronary arteries and peripheral arteries, restenoses such as after thrombolysis therapy, percutaneous transluminal angioplasty (PTA), transluminal coronary angioplasty (PTCA), reperfusion damage, endothelial dysfunction, ischaemic cardiomyopathy, renal insufficiency, nephropathies and stress-related hypertension, comprising administering an effective amount of a pharmaceutical combination according to claim 10 to a human or animal in need thereof.

13: A method for treatment or prophylaxis of acute heart failure, right heart failure, left heart failure, global failure, diabetic heart failure, heart failure with preserved ejection fraction (HFpEF), diastolic heart failure, heart failure with reduced ejection fraction (HFrEF systolic heart failure), unstable angina pectoris, myocardial ischaemia, acute coronary syndrome, NSTEMI (non-ST elevation myocardial infarction), STEMI (ST elevation myocardial infarction), ischaemic heart muscle damage, myocardial infarction, coronary microvascular dysfunction, microvascular obstruction, no-reflow phenomenon, transitory and ischaemic attacks, ischaemic and haemorrhagic stroke, peripheral and cardial vascular disorders, impaired peripheral circulation, peripheral arterial occlusive disease, primary and secondary Raynaud's syndrome, impaired microcirculation, arterial pulmonary hypertension, spasms of coronary arteries and peripheral arteries, restenoses such as after thrombolysis therapy, percutaneous transluminal angioplasty (PTA), transluminal coronary angioplasty (PTCA), reperfusion damage, endothelial dysfunction, ischaemic cardiomyopathy, renal insufficiency, nephropathies and stress-related hypertension, comprising administering an effective amount of a compound of formula (I) according to claim 4 to a human or animal in need thereof.

14: A pharmaceutical composition comprising a compound according to claim 4 in combination with one or more inert, nontoxic, pharmaceutically suitable excipients.

15: A pharmaceutical combination comprising a compound according to claim 4 in combination with one or more active compounds selected from the group of the platelet aggregation inhibitors, anticoagulants, profibrinolytic substances, substances which affect the energy metabolism of the heart and mitochondrial function/ROS production, hypotensive drugs, mineralocorticoid receptor antagonists, HMG CoA reductase inhibitors, drugs which modulate lipid metabolism, active compounds which modulate glucose metabolism and active compounds for anxiety and pain therapy such as benzodiazepines and opiates.

16: A method for treatment or prophylaxis of acute heart failure, right heart failure, left heart failure, global failure, diabetic heart failure, heart failure with preserved ejection fraction (HFpEF), diastolic heart failure, heart failure with reduced ejection fraction (HFrEF systolic heart failure), coronary heart disease, stable and unstable angina pectoris, myocardial ischaemia, acute coronary syndrome, NSTEMI (non-ST elevation myocardial infarction), STEMI (ST elevation myocardial infarction), ischaemic heart muscle damage, myocardial infarction, coronary microvascular dysfunction, microvascular obstruction, no-reflow phenomenon, transitory and ischaemic attacks, ischaemic and haemorrhagic stroke, peripheral and cardial vascular disorders, impaired peripheral circulation, peripheral arterial occlusive disease, primary and secondary Raynaud's syndrome, impaired microcirculation, arterial pulmonary hypertension, spasms of coronary arteries and peripheral arteries, restenoses such as after thrombolysis therapy, percutaneous transluminal angioplasty (PTA), transluminal coronary angioplasty (PTCA), reperfusion damage, endothelial dysfunction, ischaemic cardiomyopathy, renal insufficiency, nephropathies and stress-related hypertension, comprising administering an effective amount of a pharmaceutical composition according to claim 14 to a human or animal in need thereof.

17: A method for treatment or prophylaxis of acute heart failure, right heart failure, left heart failure, global failure, diabetic heart failure, heart failure with preserved ejection fraction (HFpEF), diastolic heart failure, heart failure with reduced ejection fraction (HFrEF systolic heart failure), coronary heart disease, stable and unstable angina pectoris, myocardial ischaemia, acute coronary syndrome, NSTEMI (non-ST elevation myocardial infarction), STEMI (ST elevation myocardial infarction), ischaemic heart muscle damage, myocardial infarction, coronary microvascular dysfunction, microvascular obstruction, no-reflow phenomenon, transitory and ischaemic attacks, ischaemic and haemorrhagic stroke, peripheral and cardial vascular disorders, impaired peripheral circulation, peripheral arterial occlusive disease, primary and secondary Raynaud's syndrome, impaired microcirculation, arterial pulmonary hypertension, spasms of coronary arteries and peripheral arteries, restenoses such as after thrombolysis therapy, percutaneous transluminal angioplasty (PTA), transluminal coronary angioplasty (PTCA), reperfusion damage, endothelial dysfunction, ischaemic cardiomyopathy, renal insufficiency, nephropathies and stress-related hypertension, comprising administering an effective amount of a pharmaceutical combination according to claim 15 to a human or animal in need thereof.
